(19) United States
(12) Patent Application Publication
(10) Pub. No.: US 2006/0241072 A1
(43) Pub. Date: Oct. 26, 2006

(54) Oligomeric compounds for use in gene modulation

(75) Inventor: Brenda F. Baker, Carisbad, CA (US)

Correspondence Address: WOODCOCK WASHBURN LLP, ONE LIBERTY PLACE - 46TH FLOOR, PHILADELPHIA, PA, 19103, US

(73) Assignee: ISIS PHARMACEUTICALS, INC.

(21) Appl. No.: 10/561,618

(22) PCT Filed: Jun. 18, 2004
(86) PCT No.: PCT/US04/19458

§ 371 Date: May 5, 2006

Related U.S. Application Data
(60) Provisional application No. 60/480,048, filed on Jun. 20, 2003.

(51) Int. Cl. A61K 48/00; C07H 21/02

(52) U.S. Cl. 514/44; 536/23.1

(57) ABSTRACT

The present invention provides modified oligomeric compounds and compositions of oligomeric compounds for use in the RNA interference pathway of gene modulation. The modified oligomeric compounds include siRNA and asRNA having at least one affinity modification. The present invention also provides methods for modulating gene expression using the modified oligomeric compounds and compositions of oligomeric compounds.

### FIELD OF THE INVENTION

The present invention provides oligomeric compounds and compositions comprising the same having sufficient complementarity and/or affinity to hybridize to a nucleic acid target and methods for their use in modulating gene expression. In one embodiment, the oligomeric compounds comprise double stranded constructs wherein the first strand is capable of hybridizing to a nucleic acid target and the second strand has sufficient complementarity to hybridize to the first strand. In some embodiments, the oligomeric compounds hybridize to a portion of a target RNA or a related nucleic acid target involved in the transcription or translation of a target RNA resulting in modulation of the activity of the target RNA.

### BACKGROUND OF THE INVENTION

In many species, introduction of double-stranded RNA (dsRNA) induces potent and specific gene silencing. This phenomenon occurs in both plants and animals and has roles in viral defense and transposon silencing mechanisms. This phenomenon was originally described more than a decade ago by researchers working with the petunia flower. While trying to deepen the purple color of these flowers, Jorgensen et al. introduced a pigment-producing gene under the control of a powerful promoter. Instead of the expected deep purple color, many of the flowers appeared variegated or even white. Jorgensen named the observed phenomenon “cosuppression,” since the expression of both the introduced gene and the homologous endogenous gene was suppressed (Napoli et al., Plant Cell, 1990, 2, 279-289; Jorgensen et al., Plant Mol. Biol., 1996, 31, 957-973).

Cosuppression has since been found to occur in many species of plants, fungi, and has been particularly well characterized in Neurospora crassa, where it is known as “quelling” (Cogoni and Macino, Genes Dev., 2000, 10, 638-643; Guru, Nature, 2000, 404, 804-808).

The first evidence that dsRNA could lead to gene silencing in animals came from work in the nematode, Caenorhabditis elegans. In 1995, researchers Guo and Kemphues were attempting to use antisense RNA to shut down expression of the par-1 gene in order to assess its function. As expected, injection of the antisense RNA disrupted expression of par-1, but quizzically, injection of the sense-strand control also disrupted expression (Guo and Kempheus, Cell, 1995, 81, 611-620). This result was a puzzle until Fire et al. injected dsRNA (a mixture of both sense and antisense strands) into C. elegans. This injection resulted in much more efficient silencing than injection of either the sense or the antisense strands alone. Injection of just a few molecules of dsRNA per cell was sufficient to completely silence the homologous gene's expression. Furthermore, injection of dsRNA into the gut of the worm caused gene silencing not only throughout the worm, but also in first generation offspring (Fire et al., Nature, 1998, 391, 806-811).

The potency of this phenomenon led Timmons and Fire to explore the limits of the dsRNA effects by feeding nematodes bacteria that had been engineered to express dsRNA homologous to the C. elegans unc-22 gene. Surprisingly, these worms developed an unc-22 null-like phenotype (Timmons and Fire, Nature 1998, 395, 854; Timmons et al., Gene, 2001, 263, 103-112). Further work showed that soaking worms in dsRNA was also able to induce silencing (Tabara et al., Science, 1998, 282, 430-431). PCT publication WO 01/48183 reports methods of inhibiting expression of a target gene in a nematode worm involving feeding to the worm a food organism which is capable of producing a double-stranded RNA structure having a nucleotide sequence substantially identical to a portion of the target gene following ingestion of the food organism by the nematode, or by introducing a DNA capable of producing the double-stranded RNA structure.

The posttranscriptional gene silencing defined in Caenorhabditis elegans resulting from exposure to double-stranded RNA (dsRNA) has since been designated as RNA interference (RNAi). This term has come to generalize all forms of gene silencing involving dsRNA leading to the sequence-specific reduction of endogenous targeted mRNA levels; unlike co-suppression, in which transgenic DNA leads to silencing of both the transgene and the endogenous gene. Introduction of exogenous double-stranded RNA (dsRNA) into Caenorhabditis elegans has been shown to specifically and potently disrupt the activity of genes containing homologous sequences. Montgomery et al. suggest that the primary interference effects of dsRNA are post-transcriptional; this conclusion being derived from examination of the primary DNA sequence after dsRNA-mediated interference a finding of no evidence of alterations followed by studies involving alteration of an upstream operon having no effect on the activity of its downstream gene. These results argue against an effect on initiation or elongation of transcription. Finally they observed by in situ hybridization, that dsRNA-mediated interference produced a substantial, although not complete, reduction in accumulation of nascent transcripts in the nucleus, while cytoplasmic accumulation of transcripts was virtually eliminated. These results indicate that the endogenous mRNA is the primary target for interference and suggest a mechanism that degrades the targeted mRNA before translation can occur. It was also found that this mechanism is not dependent on the SMG system, an mRNA surveillance system in C. elegans responsible for targeting and destroying aberrant messages. The authors further suggest a model of how dsRNA might function as a catalytic mechanism to target homologous mRNAs for degradation. (Montgomery et al., Proc. Natl. Acad. Sci. U S A, 1998, 95, 15502-15507).

Recently, the development of a cell-free system from syncytial blastoderm Drosophila embryos that recapitulates many of the features of RNAi has been reported. The interference observed in this reaction is sequence specific, is promoted by dsRNA but not single-stranded RNA, functions by specific mRNA degradation, and requires a minimum length of dsRNA. Furthermore, preincubation of dsRNA potentiates its activity demonstrating that RNAi can be mediated by sequence-specific processes in soluble reactions (Tuschl et al., Genes Dev., 1999, 13, 3191-3197).

In subsequent experiments, Tuschl et al., using the Drosophila in vitro system, demonstrated that 21- and 22-nt RNA fragments are the sequence-specific mediators of RNAi. These fragments, which they termed short interfering RNAs (siRNAs) were shown to be generated by an RNase III-like processing reaction from long dsRNA. They also showed that chemically synthesized siRNA duplexes with overhanging 3′ ends mediate efficient target RNA cleavage in the Drosophila lysate, and that the cleavage site is located near the center of the region spanned by the guiding siRNA. In addition, they suggest that the direction of dsRNA processing determines whether sense or antisense target RNA can be cleaved by the siRNA-protein complex (Elbashir et al., Genes Dev., 2001, 15, 188-200). Further characterization of the suppression of expression of endogenous and heterologous genes caused by the 21-23 nucleotide siRNAs have been investigated in several mammalian cell lines, including human embryonic kidney (293) and HeLa cells (Elbashir et al., Nature, 2001, 411, 494-498).

Most recently, Tijsterman et al., have shown that single-stranded RNA oligomers of antisense polarity can be potent inducers of gene silencing. As is the case for co-suppression, they showed that antisense RNAs act independently of the RNAi genes rde-1 and rde-4 but require the mutator/RNAi gene mut-7 and a putative DEAD box RNA helicase, mut-14. According to the authors, their data favor the hypothesis that gene silencing is accomplished by RNA primer extension using the mRNA as template, leading to dsRNA that is subsequently degraded suggesting that single-stranded RNA oligomers are ultimately responsible for the RNAi phenomenon (Tijsterman et al., Science, 2002, 295, 694-697).

Several recent publications have reported the structural requirements for the dsRNA trigger required for RNAi activity. Recent reports have indicated that ideal dsRNA sequences are 21 nt in length containing 2 nt 3′-end overhangs (Elbashir et al, EMBO, 2001, 20, 6877-6887, Sabine Brantl, Biochimica et Biophysica Acta, 2002, 1575, 15-25). In this system, substitution of the 4 nucleosides from the 3′-end with 2′-deoxynucleosides has been demonstrated to not affect activity. On the other hand, substitution with 2′ deoxynucleosides or 2′-OCH₃-nucleosides throughout the sequence (sense or antisense) was shown to be deleterious to RNAi activity.

Investigation of the structural requirements for RNA silencing in C. elegans has demonstrated modification of the internucleotide linkage (phosphorothioate) to not interfere with activity (Parrish et al., Molecular Cell, 2000, 6, 1077-1087). It was also shown by Parrish et al., that chemical modification like 2′-amino or 5′-iodouridine are well tolerated in the sense strand but not the antisense strand of the dsRNA suggesting differing roles for the 2 strands in RNAi. Base modification such as guanine to inosine (where one hydrogen bond is lost) has been demonstrated to decrease RNAi activity independently of the position of the modification (sense or antisense). Same “position independent” loss of activity has been observed following the introduction of mismatches in the dsRNA trigger. Some types of modifications, for example introduction of sterically demanding bases such as 5-iodoU, have been shown to be deleterious to RNAi activity when positioned in the antisense strand, whereas modifications positioned in the sense strand were shown to be less detrimental to RNAi activity. As was the case for the 21 nt dsRNA sequences, RNA-DNA heteroduplexes did not serve as triggers for RNAi. However, dsRNA containing 2′-2′-F modified nucleosides appeared to be efficient in triggering RNAi response independent of the position (sense or antisense) of the 2′-F modified nucleoside.

In one experiment the reduction of gene expression was studied using electroporated dsRNA and a 25mer morpholino in post implantation mouse embryos (Mellitzer et al., Mechanisms of Development, 2002, 118, 57-63). The morpholino oligomer did show activity but was not as effective as the dsRNA.

A number of PCT applications have recently been published that relate to the RNAi phenomenon. These include: PCT publication WO 00/44895, PCT publication WO 00/49035, PCT publication WO 00/63364, PCT publication WO 01/36641, PCT publication WO 01/36646, PCT publication WO 99/32619, PCT publication WO 00/44914, PCT publication WO 01/29058, and PCT publication WO 01/75164.

U.S. Pat. Nos. 5,898,031 and 6,107,094 report oligonucleotides having RNA like properties. When hybridized with RNA, these olibonucleotides serve as substrates for a dsRNase enzyme with resultant cleavage of the RNA by the enzyme.

In another recently published paper (Martinez et al., Cell, 2002, 110, 563-574) it was shown that double stranded as well as single stranded siRNA resides in the RNA-induced silencing complex (RISC) together with elF2C1 and elf2C2 (human GERp950 Argonaute proteins. The activity of 5′-phosphorylated single stranded siRNA was comparable to the double stranded siRNA in the system studied. In a related study, the inclusion of a 5′-phosphate moiety was shown to enhance activity of siRNA's in vivo in Drosophila embryos (Boutla et al., Curr. Biol., 2001, 11, 1776-1780). In another study, it was reported that the 5′-phosphate was required for siRNA function in human HeLa cells (Schwarz et al., Molecular Cell, 2002, 10, 537-548).

In one recently published paper the authors claim that inclusion of 2′-O-methyl groups into the sense, antisense or both the sense and antisense strands of a siRNA showed greatly reduced activity (Chiu et al., RNA, 2003, 9, 1034-1048). In addition, strand bias and asymmetry have been reported on in Khvorova et al., Cell, 2003, 115, 209-216 and Schwarz et al., Cell, 2003, 115, 199-208.

Like the RNAse H pathway, the RNA interference pathway of antisense modulation of gene expression is an effective means for modulating the levels of specific gene products and may therefore prove to be uniquely useful in a number of therapeutic, diagnostic, and research applications involving gene silencing. The present invention therefore further provides compositions useful for modulating gene expression pathways, including those relying on an antisense mechanism of action such as RNA interference and dsRNA enzymes as well as non-antisense mechanisms. One having skill in the art, once armed with this disclosure will be able, without undue experimentation, to identify compositions suitable for these uses.

### SUMMARY OF THE INVENTION

The present invention provides, inter alia, modified oligomeric compounds and compositions comprising the same, that can be used individually as an siRNA or combined with a second strand and used as an siRNA and methods of their use in gene modulation. The modified oligomeric compounds are modified to have at least one affinity modification.

In some embodiments, the composition comprises a first oligomeric compound or a second oligomeric compound, or both a first and second oligomeric compound. At least a portion of the first oligomeric compound is capable of hybridizing with at least a portion of the second oligomeric compound. At least a portion of the first oligomeric compound is complementary to and capable of hybridizing to a target nucleic acid. In addition, at least one of the first and the second oligomeric compounds comprises at least one modified nucleoside having enhanced or decreased affinity for the complementary nucleoside in the composition or between the first oligomeric compound and a nucleic acid target; or one of the first and the second oligomeric compounds comprises at least one modified nucleoside having enhanced affinity for the complementary nucleoside in the composition or between the first oligomeric compound and a nucleic acid target and one of the first and the second oligomeric compounds comprises at least one modified nucleoside having decreased affinity for the complementary nucleoside in the composition or between the first oligomeric compound and a nucleic acid target.

In some embodiments, the first oligomeric compound comprises at least one modified nucleoside having enhanced affinity for the complementary nucleoside in the composition or between the first oligomeric compound and a nucleic acid target and either the first oligomeric compound or second oligomeric compound comprises at least one modified nucleoside having a decreased affinity for the complementary nucleoside in the composition or between the first oligomeric compound and a nucleic acid target.

In some embodiments, the first oligomeric compound comprises at least one modified nucleoside having a decreased affinity for the complementary nucleoside in the composition or between the first oligomeric compound and a nucleic acid target, and the second oligomeric compound comprises at least one modified nucleotide having an enhanced affinity for the complementary nucleotide in the first oligomeric compound compared to the affinity of an unmodified nucleotide.

In some embodiments, the second oligomeric compound comprises at least one modified nucleotide having an enhanced affinity for the complementary nucleotide in the first oligomeric compound compared to the affinity of an unmodified nucleotide, and wherein the second oligomeric compound also comprises at least one modified nucleotide having a decreased affinity for the complementary nucleotide in the first oligomeric compound compared to the affinity of an unmodified nucleotide.

In some embodiments, the first and second oligomeric compounds are a complementary pair of siRNA oligonucleotides. In some embodiments, the first oligomeric compound is an antisense oligonucleotide and the second oligomeric compound is a sense oligonucleotide. In some embodiments, the composition further comprises at least one protein comprising at least a portion of an RNA-induced silencing complex (RISC).

In some embodiments, the at least one modified nucleotide that comprises an enhanced affinity is a nucleotide comprising a nucleotide base modification. In some embodiments, the nucleotide base modification comprises a pyrimidine nucleotide comprising a modification at the 2, 4, 5 or 6 position of the pyrimidine nucleotide. Suitable nucleotide base modifications include, for example, a 2-thio U nucleotide substitution for U nucleotide, a 2-thio C nucleotide substitution for a C nucleotide, a 5-alkyl, 5-alkenyl, or 5-alkynyl U substitution for a U nucleotide, a 5-alkyl, 5-alkenyl, or 5-alkynyl C substitution for a C nucleotide, or a 5-methyl U, 5-methyl C, 5-propynyl U, or 5-propynyl C nucleotide. In some embodiments, the nucleotide base modification comprises a pyrimidine nucleotide having a modification, wherein the pyrimidine nucleotide is incorporated as one ring of a multiple ring heterocycle. In some embodiments, the multiple ring heterocycle further comprises a phenoxazine moiety, or comprises the formula: 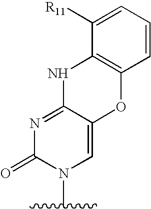
wherein R₁₁ is (CH₃)₂N—(CH₂)₂—O—, H₂N—(CH₂)₃—, Ph-CH₂—O—C(═O)—N(H)—(CH₂)₃—, H₂N—, fluorenyl-CH₂—O—C(═O)—N(H)—(CH₂)₃—, phthalimidyl-CH₂—O—C(═O)—N(H)—(CH₂)₃—, Ph-CH₂—O—C(═O)—N(H)—(CH₂)₂—O—, Ph-CH₂—O—C(═O)—N(H)—(CH₂)₃—O—, (CH₃)₂N—N(H)—(CH₂)₂—O—, fluorenyl-CH₂—O—C(—O)—N(H)—(CH₂)₂—O—, fluorenyl-CH₂—O—C(═O)—N(H)—(CH₂)₃—O—, H₂N—(CH₂)₂—O—CH₂—, N₃—(CH₂)₂—O—CH₂—, H₂N—(CH₂)₂—O—, or NH₂C(═NH)NH—.

In some embodiments, the nucleotide base modification comprises a purine nucleotide comprising a modification at the 1, 2, 3, 6, 7 or 8 position of the purine nucleotide. In some embodiments, the nucleotide base modification comprises a 7-deaza-7-alkyl, 7-deaza-7-alkenyl, or 7-deaza-7-alkynyl A substitution for a A nucleotide or 7-deaza-7-alkyl, 7-deaza-7-alkenyl, or 7-deaza-7-alkynyl G substitution for a G nucleotide. In some embodiments, the nucleotide base modification comprises a 2,6-diamino purine substitution for an A nucleotide.

In some embodiments, the at least one modified nucleotide that comprises an enhanced affinity is a nucleotide comprising a nucleotide sugar modification. Suitable nucleotide sugar modifications include, for example, 2′-F, 2′-MOE, 2′-O-methyl, 2′-O-alkyl, 2′-O-alkenyl, 2′-O-alkynyl, 2′-S-alkyl, 2′-S-alkenyl, 2′-S-alkynyl, 2′-amino, 2′-azido, and 2′-allyl.

In some embodiments, the at least one modified nucleotide that comprises an enhanced affinity is a nucleotide comprising a modified internucleotide linkage. In some embodiments, the modified internucleotide linkage comprises a stabilizing internucleotide linkage. Suitable stabilizing internucleotide linkages include, for example, a 3′-deoxy-3′-aminophosphoramidate, 3′-deoxy-3′-methylene phosphonate, 3′-deoxy-3′-aminothiophosphoramidate, acetal, thioacetal, amide-3 and amide-4, MMI, hydrazine, and morpholino.

In some embodiments, the at least one modified nucleotide that comprises a decreased affinity is a nucleotide comprising a nucleotide base modification. In some embodiments, the nucleotide base modification comprises an inosine nucleotide or a purine ribofuranosyl nucleotide.

In some embodiments, the at least one modified nucleotide that comprises a decreased affinity is a nucleotide comprising a nucleotide sugar modification. In some embodiments, the sugar modification comprises a 2′-endo sugar.

In some embodiments, the at least one modified nucleotide that comprises a decreased affinity is a nucleotide comprising at least one modified internucleotide linkage, such as a destabilizing internucleotide linkage. Suitable destabilizing internucleotide linkages include, for example, a phosphorothioate, phosphorodithioate, phosphoramidate, phosphotriester, and alkyl phosphonate internucleotide linkage.

In some embodiments, the nucleotide base modification comprises a 2′-substituent group which is, independently, F, —O—CH₂CH₂—O—CH₃, —O—C₁-C₁₂ alkyl, —O—CH₂—CH₂—CH₂—NH₂, —O—(CH₂)₂—O—N(R₁)₂, —O—CH₂C(—O)—N(R₁)₂, —O—(CH₂)₂—O—(CH₂)₂—N(R₁)₂, —O—CH₂—CH₂—CH₂—NHR₁, —O—CF₃, —N₃, —O—CH₂—CH═CH₂, —NHCOR₁, —NH₂, —NHR₁, —N(R₁)₂, —SH, —SR₁, —N(H)OH, —N(H)OR₁, —N(R₁)OH, —N(R₁)OR₁ or —O—CH₂—N(H)—C(═NR₁)(N(R₁)₂); wherein each R₁ is, independently, H, C₁-C₁₂ alkyl, a protecting group, or substituted or unsubstituted C₁-C₁₂ alkyl, C₂-C₁₂ alkenyl, or C₂-C₁₂ alkynyl, wherein the substituent groups are halogen, hydroxyl, amino, azido, cyano, haloalkyl, alkenyl, alkoxy, thioalkoxy, haloalkoxy, or aryl. In some embodiments, the at least one modified nucleotide base is a locked nucleic acid (LNA).

In some embodiments, the nucleotide sugar modification is, independently, C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—Ra), carboxyl (—C(—O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol, or a polyether of the formula (—O-alkyl)ₘₐ; wherein each Rₐ is, independently, hydrogen, a protecting group, or substituted or unsubstituted alkyl, alkenyl, or alkynyl, wherein the substituent groups are haloalkyl, alkenyl, alkoxy, thioalkoxy, haloalkoxy, aryl, halogen, hydroxyl, amino, azido, carboxy, cyano, nitro, mercapto, a sulfide group, a sulfonyl group, or a sulfoxide group; or each sugar substituent group has one of formula Ia or IIa: 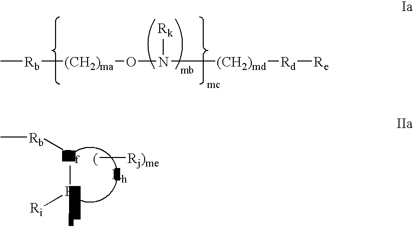
wherein R_{b} is O, S or NH; R_{d} is a single bond, O, S or C(═O); Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIa; 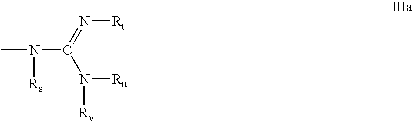
Rₚ and R_{q} are each, independently, hydrogen or C₁-C₁₀ alkyl; Rᵣ is —Rₓ-R_{y}; each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, or a conjugate group, wherein the substituent groups are hydroxyl, amino, alkoxy, carboxy, benzyl, phenyl, nitro, thiol, thioalkoxy, halogen, alkyl, aryl, alkenyl, or alkynyl; or, optionally, Rᵤ and Rᵥ, together form a phthalimido moiety with the nitrogen atom to which they are attached; each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl, or aryl; Rₓ is a bond or a linking moiety; R_{y} is a chemical functional group, a conjugate group or a solid support medium; Rₖ is hydrogen, a nitrogen protecting group or —Rₓ-R_{y}; each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are hydroxyl, amino, alkoxy, carboxy, benzyl, phenyl, nitro, thiol, thioalkoxy, halogen, alkyl, aryl, alkenyl, alkynyl, NH3+, N(Rᵤ)(Rᵥ), guanidine, or acyl where the acyl is an acid amide or an ester; or Rₘ and Rₙ, together, are a nitrogen protecting group, are joined in a ring structure that optionally includes an additional heteroatom selected from N and O, or are a chemical functional group; Rᵢ is OR_{z}, SR_{z}, or N(R_{z})₂; each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ; R_{f}, R_{g} and Rₕ comprise a ring system having from about 4 to about 7 carbon atoms or having from about 3 to about 6 carbon atoms and 1 or 2 heteroatoms wherein the heteroatoms are oxygen, nitrogen, or sulfur and wherein the ring system is aliphatic, unsaturated aliphatic, aromatic, or saturated or unsaturated heterocyclic; Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN; ma is 1 to about 10; each mb is, independently, 0 or 1; mc is 0 or an integer from 1 to 10; md is an integer from 1 to 10; me is from 0, 1 or 2; and provided that when mc is 0, md is greater than 1.

In some embodiments, each of the first and second oligomeric compounds comprises from about 8 to about 80 nucleobases, or from about 10 to about 50 nucleobases, or from about 12 or 13 to about 30 nucleobases, or from about 12 or 13 to about 24 nucleobases, or from about 19 to about 23 nucleobases.

The present invention also provides methods of inhibiting the expression of a nucleic acid molecule encoding a target protein in a cell, tissue, or animal comprising administering any of the compositions described herein to the cell, tissue, or animal, wherein the first oligomeric compound specifically hybridizes with the nucleic acid molecule encoding the target protein and inhibits the expression of the target protein.

The present invention also provides methods of screening for a modulator of a target, the method comprising contacting a target segment of a nucleic acid molecule encoding the target with one or more modulator candidates selected from the compositions described herein, and identifying one or more modulators of the target expression which modulate the expression of the target. The modulator of the target expression can comprise an oligonucleotide, an antisense oligonucleotide, a DNA oligonucleotide, an RNA oligonucleotide, an RNA oligonucleotide having at least a portion of the RNA oligonucleotide capable of hybridizing with RNA to form an oligonucleotide-RNA duplex, or a chimeric oligonucleotide.

The present invention also provides kits or assay devices comprising any of the compositions described herein.

The present invention also provides methods of treating an animal having a disease or condition associated with a target protein comprising administering to the animal a therapeutically or prophylactically effective amount of any composition described herein so that expression of the target is inhibited.

The present invention also provides methods of reducing the expression of a gene in a biological system expressing the gene comprising providing a composition described herein, and contacting the biological system with the composition under conditions effective to reduce the expression of the gene, wherein the composition comprises at least one of the first and second oligomeric compounds is an RNA oligomer.

### DESCRIPTION OF EMBODIMENTS

The present invention provides, inter alia, compositions of oligomeric compounds wherein at least a portion of the composition is double stranded region and a further portion of the composition is complementary to and hybridizes with a nucleic acid target. The compositions can comprise a single strand with regions of self-complementarity thereby forming a loop structure. The compositions can also include double-stranded compositions comprising a first and second oligomeric compound, where the first oligomeric compound hybridizes to the second oligomeric compound and further has a complementary region that hybridizes to a target nucleic acid. In this capacity the first oligomeric compound is the antisense strand and the second oligomeric compound is the sense strand of the composition.

The present invention provides modified oligomeric compounds and compositions comprising the same. These oligomers and compositions comprising the same can be used individually, for example, as an antisense RNA or combined with a second strand and used as an siRNA and methods of their use in gene modulation. The modified oligomeric compounds are modified to have at least one affinity modification.

In some embodiments, the composition comprises a first oligomeric compound or a second oligomeric compound, or both a first and second oligomeric compound. Thus, the first and second oligomers can be within separate compositions or together in the same composition. At least a portion of the first oligomeric compound is capable of hybridizing with at least a portion of the second oligomeric compound. In some embodiments, this portion comprises at least 8 nucleobases, at least 10 nucleobases, at least 12 nucleobases, at least 14 nucleobases, at least 16 nucleobases, at least 18 nucleobases, at least 20 nucleobases, at least 22 nucleobases, at least 24 nucleobases, at least 26 nucleobases, at least 28 nucleobases, or at least 30 nucleobases. In some embodiments, at least a portion of the first oligomeric compound is complementary to and capable of hybridizing to a target nucleic acid. In some embodiments, this portion comprises at least 8 nucleobases, at least 10 nucleobases, at least 12 nucleobases, at least 14 nucleobases, at least 16 nucleobases, at least 18 nucleobases, at least 20 nucleobases, at least 22 nucleobases, at least 24 nucleobases, at least 26 nucleobases, at least 28 nucleobases, or at least 30 nucleobases.

Oligomeric compounds of the present invention are affinity modified using, for the most part, chemistries know to the art skilled. Such affinity modified oligomeric compounds can be prepared having one or more modifications at one or more monomer subunit sites (nucleoside site for an oligomeric compound). Such affinity modified oligomeric compounds can be uniformly modified or modified at particular sites with the same or diverse modifications and may be modified in regions to give chimeric oligomeric compounds. The affinity of a duplex oligomeric compound can be modified to give a final duplex that has a comparable Tm to the native RNA compound in a number of ways.

For example, in some embodiments, the composition comprises a first oligomeric compound or a second oligomeric compound, or both a first and second oligomeric compound. At least a portion of the first oligomeric compound is capable of hybridizing with at least a portion of the second oligomeric compound. At least a portion of the first oligomeric compound is complementary to and capable of hybridizing to a target nucleic acid. In addition, at least one of the first and the second oligomeric compounds comprises at least one modified nucleoside having enhanced or decreased affinity for the complementary nucleoside in the composition or between the first oligomeric compound and a nucleic acid target; or one of the first and the second oligomeric compounds comprises at least one modified nucleoside having enhanced affinity for the complementary nucleoside in the composition or between the first oligomeric compound and a nucleic acid target and one of the first and the second oligomeric compounds comprises at least one modified nucleoside having decreased affinity for the complementary nucleoside in the composition or between the first oligomeric compound and a nucleic acid target.

In some embodiments, the first oligomeric compound comprises at least one modified nucleoside having enhanced affinity for the complementary nucleoside in the composition or between the first oligomeric compound and a nucleic acid target and either the first oligomeric compound or second oligomeric compound comprises at least one modified nucleoside having a decreased affinity for the complementary nucleoside in the composition or between the first oligomeric compound and a nucleic acid target.

In some embodiments, the first oligomeric compound comprises at least one modified nucleoside having a decreased affinity for the complementary nucleoside in the composition or between the first oligomeric compound and a nucleic acid target, and the second oligomeric compound comprises at least one modified nucleotide having an enhanced affinity for the complementary nucleotide in the first oligomeric compound compared to the affinity of an unmodified nucleotide.

In some embodiments, the second oligomeric compound comprises at least one modified nucleotide having an enhanced affinity for the complementary nucleotide in the first oligomeric compound compared to the affinity of an unmodified nucleotide, and wherein the second oligomeric compound also comprises at least one modified nucleotide having a decreased affinity for the complementary nucleotide in the first oligomeric compound compared to the affinity of an unmodified nucleotide.

Another way to give the same effect is to modify the affinity in a single strand of a duplex with modifications that balance each other such as increasing affinity at select sites and decreasing affinity at other selected sites. To maximize the activity of an affinity modified oligomeric comound, the positioning of selected modifications can be adjusted.

One method of modulating the affinity of complementary strands amenable to the present invention is the adjustment of the G:C (increase affinity) and the A:U (decrease affinity) percentages. One application of this method of modifying affinity would be to incorporate a high or low affinity modification in one of the strands and increase or decrease the G:C or A:U percentages. Another way to modify the affinity using a lowering of the G:C content would be to choose sequences that do not have high G:C content at the ends that would create regions of high affinity that could make it difficult for a helicase to unwind an siRNA duplex. Affinity modulating nucleosides can be placed strategically to offset regions of high or low affinity caused by high G:C or A:U regions.

Oligomeric Compounds

In the context of the present invention, the term “oligomeric compound” refers to a polymeric structure capable of hybridizing a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular and may also include branching. Oligomeric compounds can be included double stranded constructs such as for example two strands hybridized to form double stranded compounds. The double stranded oligomeric compounds can be linked or separate and can have blunt ends, overhangs on the ends or can have a combination including a blunt end and an end with an overhang. Further modifications can include conjugate groups attached to one of the termini, selected nucleobase positions, sugar positions or to one of the internucleoside linkages. In general, an oligomeric compound comprises a backbone of momeric subunits joined linking groups where each linked momeric subunit is directly or indirectly attached to a heterocyclic base moiety. Oligomeric compounds may also include monomeric subunits that are not linked to a heterocyclic base moiety thereby providing abasic sites. Any one of the repeated units making up an oligomeric compound can be modified giving rise to a variety of motifs including hemimers, gapmers and chimeras.

As is known in the art, a nucleoside comprises a sugar moiety attached to a heterocyclic base moiety. The two most common classes of such heterocyclic bases are purines and pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar giving the more common 3′, 5′-internucleoside linkage or the not so common 2′, 5′-internucleoside linkage. In forming oligonucleotides, the phosphate groups covalently link the sugar moieties of adjacent nucleosides. The respective ends can be joined to form a circular structure by hybridization or by formation of a covalent bond, however, open linear structures are generally suitable.

In the context of this invention, the term “oligonucleotide” refers to an oligomer or polymer of ribonucleic acid (RNA) or deoxyribonucleic acid (DNA). This term includes oligonucleotides composed of naturally-occurring nucleobases, sugars and covalent internucleoside linkages. The term “oligonucleotide analog” refers to oligonucleotides that have one or more non-naturally occurring portions which function in a similar manner to oligonulceotides. Such oligonucleotide analogs are often desired over the naturally occurring forms because of desirable properties such as, for example, enhanced cellular uptake, enhanced affinity for a nucleic acid target and enhanced nuclease stability.

In the context of this invention, the term “oligonucleoside” refers to a sequence of nucleosides that are joined by internucleoside linkages that do not have phosphorus atoms. Internucleoside linkages of this type include short chain alkyl, cycloalkyl, mixed heteroatom alkyl, mixed heteroatom cycloalkyl, one or more short chain heteroatomic and one or more short chain heterocyclic. These internucleoside linkages include but are not limited to siloxane, sulfide, sulfoxide, sulfone, acetyl, formacetyl, thioformacetyl, methylene formacetyl, thioformacetyl, alkeneyl, sulfamate; methyleneimino, methylenehydrazino, sulfonate, sulfonamide, amide and others having mixed N, O, S and CH₂ component parts.

Representative U.S. patents that teach the preparation of the above oligonucleosides include, but are not limited to, U.S. Pat. Nos. 5,034,506, 5,166,315, 5,185,444, 5,214,134, 5,216,141, 5,235,033, 5,264,562, 5,264,564, 5,405,938, 5,434,257, 5,466,677, 5,470,967, 5,489,677, 5,541,307, 5,561,225, 5,596,086, 5,602,240, 5,610,289, 5,602,240, 5,608,046, 5,610,289, 5,618,704, 5,623,070, 5,663,312, 5,633,360, 5,677,437, 5,792,608, 5,646,269, and 5,677,439.

Further included in the present invention are oligomeric compounds such as antisense oligomeric compounds, antisense oligonucleotides, ribozymes, external guide sequence (EGS) oligonucleotides, alternate splicers, primers, probes, and other oligomeric compounds which hybridize to at least a portion of the target nucleic acid. As such, these oligomeric compounds may be introduced in the form of single-stranded, double-stranded, circular or hairpin oligomeric compounds and may contain structural elements such as internal or terminal bulges or loops. Once introduced to a system, the oligomeric compounds of the invention may elicit the action of one or more enzymes or structural proteins to effect modification of the target nucleic acid.

One non-limiting example of such an enzyme is RNAse H, a cellular endonuclease which cleaves the RNA strand of an RNA:DNA duplex or the RNA region of a duplex that has an RNA:DNA region and may have other chemistries to enhance desired properties. It is known in the art that single-stranded antisense oligomeric compounds which are “DNA-like” elicit RNAse H. Activation of RNase H, therefore, results in cleavage of the RNA target, thereby greatly enhancing the efficiency of oligonucleotide-mediated inhibition of gene expression. Similar roles have been postulated for other ribonucleases such as those in the RNase III and ribonuclease L family of enzymes.

While one form of antisense oligomeric compound is a single-stranded antisense oligonucleotide, in many species the introduction of double-stranded constructs, such as double-stranded RNA (dsRNA) duplexes, has been shown to induce potent and specific antisense-mediated reduction of the function of a gene or its associated gene products. This phenomenon occurs in both plants and animals and is believed to have an evolutionary connection to viral defense and transposon silencing.

The oligomeric compounds in accordance with this invention can comprise from about 8 to about 80 nucleobases (i.e. from about 8 to about 80 linked nucleosides/monomeric subunits). One of ordinary skill in the art will appreciate that the invention embodies oligomeric compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases in length.

In some embodiments, the oligomeric compounds of the invention are 10 to 50 nucleobases in length. One having ordinary skill in the art will appreciate that this embodies oligomeric compounds of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases in length.

In some embodiments, the oligomeric compounds of the invention are 12 or 13 to 30 nucleobases in length. One having ordinary skill in the art will appreciate that this embodies oligomeric compounds of 12, 13, 14, 15, 16, 17, 18, 19, 20, 21, 22, 23, 24, 25, 26, 27, 28, 29, or 30 nucleobases in length.

In some embodiments, the oligomeric compounds of the invention are 12 or 13 to 24 nucleobases in length. One having ordinary skill in the art will appreciate that this embodies oligomeric compounds of 12, 13, 14, 15, 16, 17, 18, 19, 20, 21, 22, 23 or 24 nucleobases in length.

In some embodiments, the oligomeric compounds of the invention are 19 to 23 nucleobases in length. One having ordinary skill in the art will appreciate that this embodies oligomeric compounds of 19, 20, 21, 22 or 23 nucleobases in length.

Chimeric Oligomeric Compounds

It is not necessary for all positions in a oligomeric compound to be uniformly modified, and in fact more than one of the aforementioned modifications may be incorporated in a single oligomeric compound or even at a single monomeric subunit such as a nucleoside within a oligomeric compound. The present invention also includes oligomeric compounds which are chimeric oligomeric compounds. “Chimeric” oligomeric compounds or “chimeras,” in the context of this invention, are oligomeric compounds containing two or more chemically distinct regions, each made up of at least one monomer unit, i.e., a nucleotide in the case of a nucleic acid based oligomer.

Chimeric oligomeric compounds typically contain at least one region modified so as to confer increased resistance to nuclease degradation, increased cellular uptake, and/or increased binding affinity for the target nucleic acid. An additional region of the oligomeric compound may serve as a substrate for enzymes capable of cleaving RNA:DNA or RNA:RNA hybrids. By way of example, RNase H is a cellular endonuclease which cleaves the RNA strand of an RNA:DNA duplex. Activation of RNase H, therefore, results in cleavage of the RNA target, thereby greatly enhancing the efficiency of inhibition of gene expression. Consequently, comparable results can often be obtained with shorter oligomeric compounds when chimeras are used, compared to for example phosphorothioate deoxyoligonucleotides hybridizing to the same target region. Cleavage of the RNA target can be routinely detected by gel electrophoresis and, if necessary, associated nucleic acid hybridization techniques known in the art.

Chimeric oligomeric compounds of the invention may be formed as composite structures of two or more oligonucleotides, oligonucleotide analogs, oligonucleosides and/or oligonucleotide mimetics as described above. Routinely used chimeric compounds include but are not limited to hybrid, hemimers, gapmers, inverted gapmers and blockmers wherein the various point modifications and or regions are selected from native or modified DNA and RNA type units and or mimetic type subunits such as for example LNA, ENA, PNA, morpholinos, and others. Representative U.S. patents that teach the preparation of such hybrid structures include, but are not limited to, U.S. Pat. Nos. 5,013,830, 5,149,797, 5,220,007, 5,256,775, 5,366,878, 5,403,711, 5,491,133, 5,565,350, 5,623,065, 5,652,355, 5,652,356, and 5,700,922.

Oligomer Mimetics

Another group of oligomeric compounds amenable to the present invention includes oligonucleotide mimetics. The term “mimetic” as it is applied to oligonucleotides is intended to include oligomeric compounds wherein the furanose ring or the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid.

One such oligomeric compound, an oligonucleotide mimetic that has been shown to have excellent hybridization properties, is referred to as a peptide nucleic acid (PNA). PNAs have favorable hybridization properties, high biological stability and are electrostatically neutral molecules. In one recent study PNAs were used to correct aberrant splicing in a transgenic mouse model (Sazani et al., Nat. Biotechnol., 2002, 20, 1228-1233). In PNA oligomeric compounds, the sugar-backbone of an oligonucleotide is replaced with an amide containing backbone, in particular an aminoethylglycine backbone. The nucleobases are bound directly or indirectly (—C(═O)—CH₂— as shown below) to aza nitrogen atoms of the amide portion of the backbone. Representative U.S. patents that teach the preparation of PNA oligomeric compounds include, but are not limited to, U.S. Pat. Nos. 5,539,082, 5,714,331, and 5,719,262. PNAs can be obtained commercially from Applied Biosystems (Foster City, Calif., USA).

Numerous modifications have been made to the basic PNA backbone since it was introduced in 1991 by Nielsen and coworkers (Nielsen et al., Science, 1991, 254, 1497-1500). The basic structure is shown below: 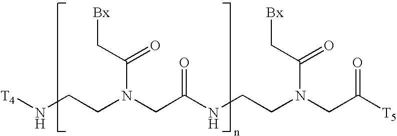
wherein:

Bx is a heterocyclic base moiety;

T₄ is hydrogen, an amino protecting group, —C(O)R₅, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, a reporter group, a conjugate group, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group, wherein the substituent groups are hydroxyl, amino, alkoxy, carboxy, benzyl, phenyl, nitro, thiol, thioalkoxy, halogen, alkyl, aryl, alkenyl, or alkynyl;

T₅ is —OH, —N(Z₁)Z₂, R₅, D or L α-amino acid linked via the α-amino group or optionally through the ω-amino group when the amino acid is lysine or ornithine or a peptide derived from D, L or mixed D and L amino acids linked through an amino group, a chemical functional group, a reporter group, or a conjugate group;

Z₁ is hydrogen, C₁-C₆ alkyl, or an amino protecting group;

Z₂ is hydrogen, C₁-C₆ alkyl, an amino protecting group, —C(═O)—(CH₂)ₙ-J-Z₃, a D or L α-amino acid linked via the α-carboxyl group or optionally through the ω-carboxyl group when the amino acid is aspartic acid or glutamic acid or a peptide derived from D, L or mixed D and L amino acids linked through a carboxyl group;

Z₃ is hydrogen, an amino protecting group, —C₁-C₆ alkyl, C(═O)—CH₃, benzyl, benzoyl, or —(CH₂)ₙ—N(H)Z₁;

each J is O, S or NH;

R₅ is a carbonyl protecting group; and

n is from 2 to about 50.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups have been selected to give a non-ionic oligomeric compound. The non-ionic morpholino-based oligomeric compounds are less likely to have undesired interactions with cellular proteins. Morpholino-based oligomeric compounds are non-ionic mimics of oligonucleotides which are less likely to form undesired interactions with cellular proteins (Dwaine A. Braasch and David R. Corey, Biochemistry, 2002, 41(14), 4503-4510). Morpholino-based oligomeric compounds have been studied in ebrafish embryos (see, Genesis, volume 30, issue 3, 2001 and Heasman, J., Dev. Biol., 2002, 243, 209-214). Further studies of Morpholino-based oligomeric compounds have also been reported (see: Nasevicius et al., Nat. Genet., 2000, 26, 216-220; and Lacerra et al., Proc. Natl. Acad. Sci., 2000, 97, 9591-9596). Morpholino-based oligomeric compounds are disclosed in U.S. Pat. No. 5,034,506. The morpholino class of oligomeric compounds have been prepared having a variety of different linking groups joining the monomeric subunits.

Morpholino nucleic acids have been prepared having a variety of different linking groups (L₂) joining the monomeric subunits. The basic formula is shown below: 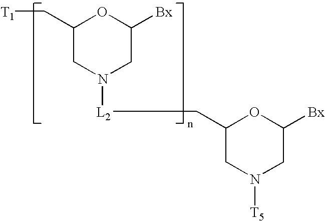
wherein:

T₁ is hydrogen, hydroxyl, a protected hydroxyl, a linked nucleoside or a linked oligomeric compound;

T₅ is hydrogen or a phosphate, phosphate derivative, a linked nucleoside or a linked oligomeric compound; and

L₂ is a linking group which can be varied from chiral to achiral from charged to neutral (U.S. Pat. No. 5,166,315 discloses linkages including —O—P(═O)(N(CH₃)₂)—O—; U.S. Pat. No. 5,034,506 discloses achiral intermorpholino linkages such as for example: —S(═O)—X— where X is NH, NCH₃, O, S, or CH₂; —C(═Y)—O— where Y is O or S; —S(═O)(OH)—CH₂—; —S(═O)(OH)—N(R)—CH₂— where R is H or CH₃; and U.S. Pat. No. 5,185,444 discloses phosphorus containing chiral intermorpholino linkages such as for example: —P(═O)(—X)—O— where X is F, CH₂R, S—CH²R, or NR₁R₂ and each R, R₁ and R₂ is H, CH₃ or some other moiety that does not interfer with the base specific hydrogen bonding; and

n is from 2 to about 50.

A further class of oligonucleotide mimetic is referred to as cyclohexenyl nucleic acids (CeNA). The furanose ring normally present in an DNA/RNA molecule is replaced with a cyclohenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (see Wang et al., J. Am. Chem. Soc., 2000, 122, 8595-8602). In general, the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. coli RNase resulting in cleavage of the target RNA strand.

The general formula of CeNA is shown below: 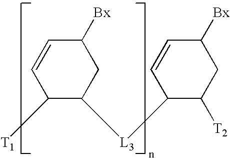
wherein:

each Bx is a heterocyclic base moiety;

L₃ is an inter cyclohexenyl linkage such as for example a phosphodiester or a phosphorothioate linkage;

T₁ is hydrogen, hydroxyl, a protected hydroxyl, a linked nucleoside or a linked oligomeric compound; and

T₂ is hydrogen or a phosphate, phosphate derivative, a linked nucleoside or a linked oligomeric compound.

Another class of oligonucleotide mimetic (anhydrohexitol nucleic acid) can be prepared from one or more anhydrohexitol nucleosides (see, Wouters and Herdewijn, Bioorg. Med. Chem. Lett., 1999, 9, 1563-1566) and would have the general formula: 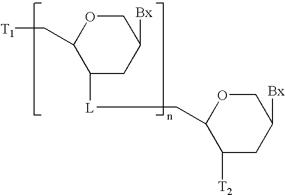
each Bx is a heterocyclic base moiety;

L is an inter anhydrohexitol linkage such as for example a phosphodiester or a phosphorothioate linkage;

T₁ is hydrogen, hydroxyl, a protected hydroxyl, a linked nucleoside or a linked oligomeric compound; and

T₂ is hydrogen or a phosphate, phosphate derivative, a linked nucleoside or a linked oligomeric compound.

Another modification includes bicyclic sugar moieties such as “Locked Nucleic Acids” (LNAs) in which the 2′-hydroxyl group of the ribosyl sugar ring is linked to the 4′ carbon atom of the sugar ring thereby forming a 2′-C,4′-C-oxymethylene linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—)ₙ group bridging the 2′ oxygen atom and the 4′ carbon atom for n=1 the term LNA (locked nucleic acid used here for 2′-O,4′-methylene-bridged nucleic acid) is used for n=2 the term ENATM (2′-O,4′-ethylene-bridged nucleic acid) is used (Singh et al., Chem. Commun., 1998, 4, 455-456; ENA: Morita et al., Bioorganic Medicinal Chemistry, 2003, 11, 2211-2226). LNA and other bicyclic sugar analogs display very high duplex thermal stabilities with complementary DNA and RNA (Tm=+3 to +10 C), stability towards 3′-exonucleolytic degradation and good solubility properties. LNAs are commercially available from ProLigo (Paris, France and Boulder, Colo., USA). The basic structure of LNA showing the bicyclic ring system is shown below: 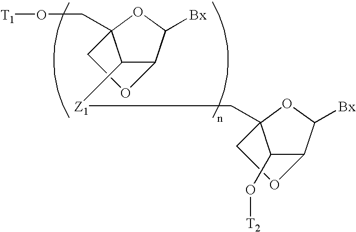
wherein:

each T₁ and T₂ is, independently, hydrogen, a hydroxyl protecting group, a linked nucleoside or a linked oligomeric compound, and each Z₁ is an internucleoside linking group such as for example phosphodiester or phosphorothioate.

An isomer of LNA that has also been studied is -L-LNA which has been shown to have superior stability against a 3′-exonuclease (Frieden et al., Nucleic Acids Research, 2003, 21, 6365-6372). The -L-LNAs were incorporated into antisense gapmers and chimeras that showed potent antisense activity. The structure of -L-LNA is shown below:

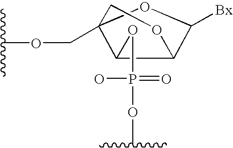

Another similar bicyclic sugar moiety that has been prepared and studied has the bridge going from the 3′-hydroxyl group via a single methylene group to the 4′ carbon atom of the sugar ring thereby forming a 3′-C,4′-C-oxymethylene linkage (see U.S. Pat. No. 6,043,060).

The conformations of LNAs determined by 2D NMR spectroscopy have shown that the locked orientation of the LNA nucleotides, both in single-stranded LNA and in duplexes, constrains the phosphate backbone in such a way as to introduce a higher population of the N-type conformation (Petersen et al., J. Mol. Recognit., 2000, 13, 44-53). These conformations are associated with improved stacking of the nucleobases (Wengel et al., Nucleosides Nucleotides, 1999, 18, 1365-1370).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands. DNA.LNA chimeras have been shown to efficiently inhibit gene expression when targeted to a variety of regions (5′-untranslated region, region of the start codon or coding region) within the luciferase mRNA (Braasch et al., Nucleic Acids Research, 2002, 30, 5160-5167).

Novel types of LNA-oligomeric compounds, as well as the LNAs, are useful in a wide range of diagnostic and therapeutic applications. Among these are antisense applications, PCR applications, strand-displacement oligomers, substrates for nucleic acid polymerases and generally as nucleotide based drugs.

Potent and nontoxic antisense oligonucleotides containing LNAs have been described (Wahlestedt et al., Proc. Natl. Acad. Sci. U.S.A., 2000, 97, 5633-5638.) The authors have demonstrated that LNAs confer several desired properties to antisense compounds. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished. Further successful in vivo studies involving LNAs have shown knock-down of the rat delta opioid receptor without toxicity (Wahlestedt et al., Proc. Natl. Acad. Sci., 2000, 97, 5633-5638) and in another study showed a blockage of the translation of the large subunit of RNA polymerase II (Fluiter et al., Nucleic Acids Res., 2003, 31, 953-962).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

The first analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (PCT International Application WO 98-DK393 19980914). Furthermore, synthesis of 2′-amino-LNA, a novel conformationally restricted high-affinity oligonucleotide analog with a handle has been described in the art (Singh et al., J. Org. Chem., 1998, 63, 10035-10039). In addition, 2′-Amino- and 2′-methylamino-LNAs have been prepared and the thermal stability of their duplexes with complementary RNA and DNA strands has been previously reported.

Another oligonucleotide mimetic amenable to the present invention that has been prepared and studied is threose nucleic acid. This oligonucleotide mimetic is based on threose nucleosides instead of ribose nucleosides and has the general structure shown below: 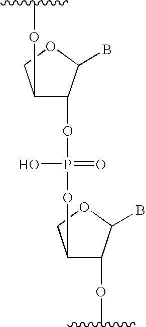
Initial interest in (3′,2′)-L-threose nucleic acid (TNA) was directed to the question of whether a DNA polymerase existed that would copy the TNA. It was found that certain DNA polymerases are able to copy limited stretches of a TNA template (reported in C&EN/Jan. 13, 2003).

In another study it was determined that TNA is capable of antiparallel Watson-Crick base pairing with complementary DNA, RNA and TNA oligonucleotides (Chaput et al., J. Am. Chem. Soc., 2003, 125, 856-857).

In one study (3′,2′)-L-threose nucleic acid was prepared and compared to the 2′ and 3′ amidate analogs (Wu et al., Organic Letters, 2002, 4(8), 1279-1282). The amidate analogs were shown to bind to RNA and DNA with comparable strength to that of RNA/DNA.

Further oligonucleotide mimetics have been prepared to incude bicyclic and tricyclic nucleoside analogs having the formulas (amidite monomers shown): 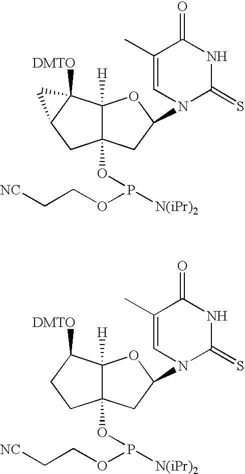
(see Steffens et al., Helv. Chim. Acta, 1997, 80, 2426-2439; Steffens et al., J. Am. Chem. Soc., 1999, 121, 3249-3255; Renneberg et al., J. Am. Chem. Soc., 2002, 124, 5993-6002; and Renneberg et al., Nucleic acids res., 2002, 30, 2751-2757). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself. Oligomeric compounds containing bicyclic nucleoside analogs have shown thermal stabilities approaching that of DNA duplexes.

Another class of oligonucleotide mimetic is referred to as phosphonomonoester nucleic acids which incorporate a phosphorus group in the backbone. This class of olignucleotide mimetic is reported to have useful physical and biological and pharmacological properties in the areas of inhibiting gene expression (antisense oligonucleotides, ribozymes, sense oligonucleotides and triplex-forming oligonucleotides), as probes for the detection of nucleic acids and as auxiliaries for use in molecular biology.

The general formula (for definitions of Markush variables see: U.S. Pat. Nos. 5,874,553 and 6,127,346) is shown below.

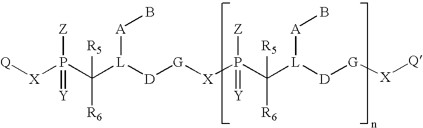

Further oligonucleotide mimetics amenable to the present invention have been prepared wherein a cyclobutyl ring replaces the naturally occurring furanosyl ring.

Oligomer and Monomer Modifications

As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound, however, linear compounds are generally desired. In addition, linear compounds may have internal nucleobase complementarity and may therefore fold in a manner as to produce a fully or partially double-stranded compound. Within oligonucleotides, the phosphate groups are commonly referred to as forming the internucleoside linkage or in conjunction with the sugar ring the backbone of the oligonucleotide. The normal internucleoside linkage that makes up the backbone of RNA and DNA is a 3′ to 5′ phosphodiester linkage.

Modified Internucleoside Linkages

Specific examples of antisense oligomeric compounds useful in this invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

In the C. elegans system, modification of the internucleotide linkage (phosphorothioate) did not significantly interfere with RNAi activity. Based on this observation, it is suggested that certain oligomeric compounds of the invention can also have one or more modified internucleoside linkages. A suitable phosphorus containing modified internucleoside linkage is the phosphorothioate internucleoside linkage.

Suitable modified oligonucleotide backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, phosphonoacetate and thiophosphonoacetate (see Sheehan et al., Nucleic Acids Research, 2003, 31(14), 4109-4118; and Dellinger et al., J. Am. Chem. Soc., 2003, 125, 940-950), selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Suitable oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

N3′-P5′-phosphoramidates have been reported to exhibit both a high affinity towards a complementary RNA strand and nuclease resistance (Gryaznov et al., J. Am. Chem. Soc., 1994, 116, 3143-3144). N3′-P5′-phosphoramidates have been studied with some success in vivo to specifically down regulate the expression of the c-myc gene (Skorski et al., Proc. Natl. Acad. Sci., 1997, 94, 3966-3971; and Faira et al., Nat. Biotechnol., 2001, 19, 40-44).

Representative U.S. patents that teach the preparation of the above phosphorus-containing linkages include, but are not limited to, U.S. Pat. Nos. 3,687,808, 4,469,863, 4,476,301, 5,023,243, 5,177,196, 5,188,897, 5,264,423, 5,276,019, 5,278,302, 5,286,717, 5,321,131, 5,399,676, 5,405,939, 5,453,496, 5,455,233, 5,466,677, 5,476,925, 5,519,126, 5,536,821, 5,541,306, 5,550,111, 5,563,253, 5,571,799, 5,587,361, 5,194,599, 5,565,555, 5,527,899, 5,721,218, 5,672,697, and 5,625,050.

In other embodiments of the invention, oligomeric compounds have one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Suitable amide internucleoside linkages are disclosed in the above referenced U.S. Pat. No. 5,602,240.

Suitable modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above oligonucleosides include, but are not limited to, U.S. Pat. Nos. 5,034,506, 5,166,315, 5,185,444, 5,214,134, 5,216,141, 5,235,033, 5,264,562, 5,264,564, 5,405,938, 5,434,257, 5,466,677, 5,470,967, 5,489,677, 5,541,307, 5,561,225, 5,596,086, 5,602,240, 5,610,289, 5,602,240, 5,608,046, 5,610,289, 5,618,704, 5,623,070, 5,663,312, 5,633,360, 5,677,437, 5,792,608, 5,646,269, and 5,677,439.

Modified Sugars

Oligomeric compounds of the invention may also contain one or more substituted or other wise modified sugar moieties. Ribosyl and related sugar moieties are routinely modified at any reactive position not involved in linking. Thus, a suitable position for a sugar substituent group is the 2′-position not usually used in the native 3′ to 5′-internucleoside linkage. Other suitable positions are the 3′ and the 5′-termini. 3′-sugar positions are open to modification when the linkage between two adjacent sugar units is a 2′,5′-linkage. Suitable sugar substituent groups include: OH; F; O-, S-, or N-alkyl; O-, S-, or N-alkenyl; O—, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other suitable oligonucleotides comprise a sugar substituent group selected from: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties.

A suitable modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Additional modifications include, but are not limited to, 2′-dimethylaminooxyethoxy, i.e., a O(CH₂)₂ON(CH₃)₂ group, also known as 2′-DMAOE, as described in examples hereinbelow, 2′-dimethylaminoethoxyethoxy (also known in the art as 2′-O-dimethyl-amino-ethoxy-ethyl or 2′-DMAEOE), i.e., 2′-O—(CH₂)₂O—(CH₂)₂N(CH₃)₂, and N-methylacetamide (also referred to as NMA, 2′-O—CH₂—C(═O)—N(H)CH₃.)

Other suitable sugar substituent groups include methoxy (—O—CH₃), aminopropoxy (—OCH₂CH₂CH₂NH₂), allyl (—CH₂—CH═CH₂), —O-allyl (—O—CH₂—CH═CH₂) and fluoro (F). 2′-Sugar substituent groups may be in the arabino (up) position or ribo (down) position. A suitable 2′-arabino modification is 2′-F (see: Loc et al., Biochemistry, 2002, 41, 3457-3467). Similar modifications may also be made at other positions on the oligomeric compound, particularly the 3′ position of the sugar on the 3′ terminal nucleoside or in 2′-5′ linked oligonucleotides and the 5′ position of 5′ terminal nucleotide. Oligomeric compounds may also have sugar mimetics such as cyclobutyl moieties in place of the pentofuranosyl sugar. Representative U.S. patents that teach the preparation of such modified sugar structures include, but are not limited to, U.S. Pat. Nos. 4,981,957, 5,118,800, 5,319,080, 5,359,044, 5,393,878, 5,446,137, 5,466,786, 5,514,785, 5,519,134, 5,567,811, 5,576,427, 5,591,722, 5,597,909, 5,610,300, 5,627,053, 5,639,873, 5,646,265, 5,658,873, 5,670,633, 5,792,747, 5,700,920, and 6,147,200.

Further representative sugar substituent groups include groups of formula Ia or IIa: 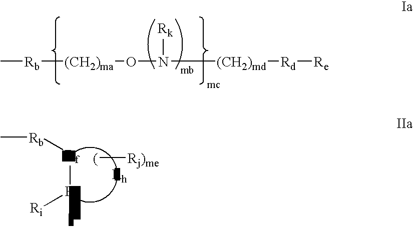
wherein:

R_{b} is O, S or NH;

R_{d} is a single bond, O, S or C(═O);

Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIa;

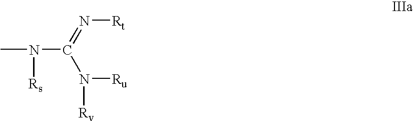

Rₚ and R_{q} are each independently hydrogen or C₁-C₁₀ alkyl;

Rᵣ is —Rₓ-R_{y};

each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group or a conjugate group, wherein the substituent groups are hydroxyl, amino, alkoxy, carboxy, benzyl, phenyl, nitro, thiol, thioalkoxy, halogen, alkyl, aryl, alkenyl, or alkynyl;

or optionally, Rᵤ and Rᵥ, together form a phthalimido moiety with the nitrogen atom to which they are attached;

each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl, or aryl;

Rₖ is hydrogen, a nitrogen protecting group or —Rₓ-R_{y};

Rₓ is a bond or a linking moiety;

R_{y} is a chemical functional group, a conjugate group or a solid support medium;

each Rₘ and Rₙ is, independently, H, a nitrogen protecting group, substituted or unsubstituted C₁-C₁₀ alkyl, substituted or unsubstituted C2-C10 alkenyl, substituted or unsubstituted C₂-C₁₀ alkynyl, wherein the substituent groups are hydroxyl, amino, alkoxy, carboxy, benzyl, phenyl, nitro, thiol, thioalkoxy, halogen, alkyl, aryl, alkenyl, alkynyl; NH3+, N(Rᵤ)(Rᵥ), guanidine, or acyl where the acyl is an acid amide or an ester;

or Rₖ and Rₘ, together, are a nitrogen protecting group, are joined in a ring structure that optionally includes an additional heteroatom selected from N and O or are a chemical functional group;

Rᵢ is OR_{z}, SR_{z}, or N(R_{z})₂;

each R_{z} is, independently, H, C₁-C₈ alkyl, C₁-C₈ haloalkyl, C(═NH)N(H)Rᵤ, C(—O)N(H)Rᵤ or OC(═O)N(H)Rᵤ;

R_{f}, R_{g} and Rₕ comprise a ring system having from about 4 to about 7 carbon atoms or having from about 3 to about 6 carbon atoms and 1 or 2 heteroatoms wherein the heteroatoms are oxygen, nitrogen, or sulfur and wherein the ring system is aliphatic, unsaturated aliphatic, aromatic, or saturated or unsaturated heterocyclic;

Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;

ma is 1 to about 10;

each mb is, independently, 0 or 1;

mc is 0 or an integer from 1 to 10;

md is an integer from 1 to 10;

me is from 0, 1 or 2; and

provided that when mc is 0, md is greater than 1.

Representative substituents groups of Formula I are disclosed in U.S. patent application Ser. No. 09/130,973, filed Aug. 7, 1998, entitled “Capped 2′ Oxyethoxy Oligonucleotides.”

Representative cyclic substituent groups of Formula II are disclosed in U.S. patent application Ser. No. 09/123,108, filed Jul. 27, 1998, entitled “RNA Targeted 2′ Oligomeric compounds that are Conformationally Preorganized.”

Additional sugar substituent groups include O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃))₂, where n and m are from 1 to about 10.

Representative guanidino substituent groups that are shown in formula III and IV are disclosed in co-owned U.S. patent application Ser. No. 09/349,040, entitled “Functionalized Oligomers” filed Jul. 7, 1999.

Representative acetamido substituent groups are disclosed in U.S. Pat. No. 6,147,200.

Representative dimethylaminoethyloxyethyl substituent groups are disclosed in International Patent Application PCT/US99/17895, entitled “2′-O-Dimethylaminoethyloxyethyl-Oligomeric compounds” filed Aug. 6, 1999.

Modified Nucleobases/Naturally Occurring Nucleobases

Oligomeric compounds may also include nucleobase (often referred to in the art simply as “base” or “heterocyclic base moiety”) modifications or substitutions. As used herein, “unmodified” or “natural” nucleobases include the purine bases adenine (A) and guanine (G), and the pyrimidine bases thymine (T), cytosine (C) and uracil (U). Modified nucleobases also referred herein as heterocyclic base moieties include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH3) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine.

Heterocyclic base moieties may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are particularly useful for increasing the binding affinity of the oligomeric compounds of the invention. These include 5-substituted pyrimidines, 6-azapyrimidines and N-2, N-6 and O-6 substituted purines, including 2-aminopropyladenine, 5-propynyluracil and 5-propynylcytosine. 5 methylcytosine substitutions have been shown to increase nucleic acid duplex stability by 0.6-1.2° C. (Sanghvi, Y. S., Crooke, S. T. and Lebleu, B., eds., Antisense Research and Applications, CRC Press, Boca Raton, 1993, pp. 276-278) and are presently suitable base substitutions, even more particularly when combined with 2′-O-methoxyethyl sugar modifications.

Oligomeric compounds of the present invention can also include polycyclic heterocyclic compounds in place of one or more heterocyclic base moieties. A number of tricyclic heterocyclic comounds have been previously reported. These compounds are routinely used in antisense applications to increase the binding properties of the modified strand to a target strand. The most studied modifications are targeted to guanosines hence they have been termed G-clamps or cytidine analogs. Many of these polycyclic heterocyclic compounds have the general formula:

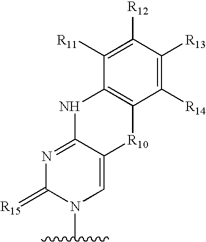

Representative cytosine analogs that make 3 hydrogen bonds with a guanosine in a second strand include 1,3-diazaphenoxazine-2-one (R₁₀═O, R₁₁-R₁₄═H) (Kurchavov et al., Nucleosides and Nucleotides, 1997, 16, 1837-1846), 1,3-diazaphenothiazine-2-one (R₁₀═S, R₁₁-R₁₄═H), (Lin et al., J. Am. Chem. Soc., 1995, 117, 3873-3874) and 6,7,8,9-tetrafluoro-1,3-diazaphenoxazine-2-one (R₁₀═O, R₁₁-R₁₄═F) (Wang et al., Tetrahedron Lett., 1998, 39, 8385-8388). Incorporated into oligonucleotides these base modifications were shown to hybridize with complementary guanine and the latter was also shown to hybridize with adenine and to enhance helical thermal stability by extended stacking interactions(also see U.S. patent application entitled “Modified Peptide Nucleic Acids” filed May 24, 2002, Ser. No. 10/155,920; and U.S. patent application entitled “Nuclease Resistant Chimeric Oligonucleotides” filed May 24, 2002, Ser. No. 10/013,295).

Further helix-stabilizing properties have been observed when a cytosine analog/substitute has an aminoethoxy moiety attached to the rigid 1,3-diazaphenoxazine-2-one scaffold (R₁₀═O, R₁₁═—O—(CH₂)₂—NH₂, R₁₂-R₁₄═H) (Lin et al., J. Am. Chem. Soc., 1998, 120, 8531-8532). Binding studies demonstrated that a single incorporation could enhance the binding affinity of a model oligonucleotide to its complementary target DNA or RNA with a ΔTm of up to 18° relative to 5-methyl cytosine dC5^{me}, which is the highest known affinity enhancement for a single modification, yet. On the other hand, the gain in helical stability does not compromise the specificity of the oligonucleotides. The Tm data indicate an even greater discrimination between the perfect match and mismatched sequences compared to dC5^{me}. It was suggested that the tethered amino group serves as an additional hydrogen bond donor to interact with the Hoogsteen face, namely the O6, of a complementary guanine thereby forming 4 hydrogen bonds. This means that the increased affinity of G-clamp is mediated by the combination of extended base stacking and additional specific hydrogen bonding.

Further tricyclic heterocyclic compounds and methods of using them that are amenable to the present invention are disclosed in U.S. Pat. No. 6,028,183 and U.S. Pat. No. 6,007,992.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNaseH, enhance cellular uptake and exhibit an increased antisense activity (Lin et al., Am. Chem. Soc., 1998, 120, 8531-8532). The activity enhancement was even more pronounced in case of G-clamp, as a single substitution was shown to significantly improve the in vitro potency of a 20mer 2′-deoxyphosphorothioate oligonucleotides (Flanagan et al., Proc. Natl. Acad. Sci. USA, 1999, 96, 3513-3518). Nevertheless, to optimize oligonucleotide design and to better understand the impact of these heterocyclic modifications on the biological activity, it is important to evaluate their effect on the nuclease stability of the oligomers.

Further modified polycyclic heterocyclic compounds useful as heterocyclcic bases are disclosed in but not limited to, the above noted U.S. Pat. No. 3,687,808, as well as U.S. Pat. Nos. 4,845,205, 5,130,302, 5,134,066, 5,175,273, 5,367,066, 5,432,272, 5,434,257, 5,457,187, 5,459,255, 5,484,908, 5,502,177, 5,525,711, 5,552,540, 5,587,469, 5,594,121, 5,596,091, 5,614,617, 5,645,985, 5,646,269, 5,750,692, 5,830,653, 5,763,588, 6,005,096, and 5,681,941, and U.S. patent application Ser. No. 09/996,292 filed Nov. 28, 2001.

The effect of many of the modifications described herein on affinity is disclosed in, for example, Freier et al., Nuc. Acids Res., 1997, 25, 4429-4443.

Conjugates

Oligomeric compounds used in the compositions of the present invention can also be modified to have one or more moieties or conjugates for enhancing the activity, cellular distribution or cellular uptake of the resulting oligomeric compounds. In one embodiment such modified oligomeric compounds are prepared by covalently attaching conjugate groups to functional groups such as hydroxyl or amino groups. Conjugate groups of the invention include, but are not limited to, intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include, but are not limited to, cholesterols, lipids, phospholipids, biotin, phenazine, folate, phenanthridine, anthraquinone, acridine, fluoresceins, rhodamines, coumarins, and dyes such as including Cy3 and Alexa. Groups that enhance the pharmacodynamic properties, in the context of this invention, include groups that improve oligomer uptake, enhance oligomer resistance to degradation, and/or strengthen sequence-specific hybridization with RNA. Groups that enhance the pharmacokinetic properties, in the context of this invention, include groups that improve oligomer uptake, distribution, metabolism or excretion. Representative conjugate groups are disclosed in International Patent Application PCT/US92/09196, filed Oct. 23, 1992.

Conjugate moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-oxycholesterol moiety (Crooke et al., J. Pharmacol. Exp. Ther., 1996, 277, 923-937).

The oligomeric compounds of the invention may also be conjugated to active drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indomethicin, a barbiturate, a cephalosporin, a sulfa drug, an antidiabetic, an antibacterial or an antibiotic. Oligonucleotide-drug conjugates and their preparation are described in U.S. patent application Ser. No. 09/334,130 (filed Jun. 15, 1999).

Representative U.S. patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Oligomeric compounds used in the compositions of the present invention can also be modified to have one or more stabilizing groups that are generally attached to one or both termini of oligomeric compounds to enhance properties such as for example nuclease stability. Included in stabilizing groups are cap structures. By “cap structure or terminal cap moiety” is meant chemical modifications, which have been incorporated at either terminus of oligonucleotides (see for example Wincott et al., WO 97/26270). These terminal modifications protect the oligomeric compounds having terminal nucleic acid molecules from exonuclease degradation, and can help in delivery and/or localization within a cell. The cap can be present at the 5′-terminus (5′-cap) or at the 3′-terminus (3′-cap) or can be present on both termini. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270).

Suitable 3′-cap structures of the present invention include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

Additional 3′ and 5′-stabilizing groups that can be used to cap one or both ends of an oligomeric compound to impart nuclease stability include those disclosed in WO 03/004602 published on Jan. 16, 2003.

3′-Endo Modifications

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA like nucleosides so that particular properties of an oligomeric compound can be enhanced while maintaining the desirable 3′-endo conformational geometry. There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C. elegans system. Properties that are enhanced by using more stable 3′-endo nucleosides include, but are not limited to, modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. The present invention provides oligomeric triggers of RNAi having one or more nucleosides modified in such a way as to favor a C3′-endo type conformation.

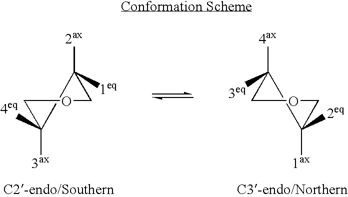

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions. (Principles of Nucleic Acid Structure, Wolfgang Sanger, 1984, Springer-Verlag.) Modification of the 2′ position to favor the 3′-endo conformation can be achieved while maintaining the 2′-OH as a recognition element (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, oligomeric triggers of RNAi response might be composed of one or more nucleosides modified in such a way that conformation is locked into a C3′-endo type conformation, i.e. Locked Nucleic Acid DNA, Singh et al, Chem. Commun. (1998), 4, 455-456), and ethylene bridged Nucleic Acids (ENA, Morita et al, Bioorganic & Medicinal Chemistry Letters (2002), 12, 73-76.)

A preferred conformation of modified nucleosides and their oligomers can be estimated by various methods such as molecular dynamics calculations, nuclear magnetic resonance spectroscopy and CD measurements. Hence, modifications predicted to induce RNA like conformations, A-form duplex geometry in an oligomeric context, are selected for use in the modified oligonucleotides of the present invention. The synthesis of numerous of the modified nucleosides amenable to the present invention are known in the art (see for example, Chemistry of Nucleosides and Nucleotides Vol 1-3, ed. Leroy B. Townsend, 1988, Plenum press., and the examples section below.)

In one aspect, the present invention is directed to oligomers that are prepared having enhanced properties compared to native RNA against nucleic acid targets. A target is identified and an oligomer is selected having an effective length and sequence that is complementary to a portion of the target sequence. Each nucleoside of the selected sequence is scrutinized for possible enhancing modifications. A suitable modification would be the replacement of one or more RNA nucleosides with nucleosides that have the same 3′-endo conformational geometry. Such modifications can enhance chemical and nuclease stability relative to native RNA while at the same time being much cheaper and easier to synthesize and/or incorporate into an oligonucleotide. The selected sequence can be further divided into regions and the nucleosides of each region evaluated for enhancing modifications that can be the result of a chimeric configuration. Consideration is also given to the 5′ and 3′-termini as there are often advantageous modifications that can be made to one or more of the terminal nucleosides. The oligomeric compounds of the present invention include at least one 5′-modified phosphate group on a single strand or on at least one 5′-position of a double stranded sequence or sequences. Further modifications are also considered such as internucleoside linkages, conjugate groups, substitute sugars or bases, substitution of one or more nucleosides with nucleoside mimetics and any other modification that can enhance the selected sequence for its intended target.

The terms used to describe the conformational geometry of homoduplex nucleic acids are “A Form” for RNA and “B Form” for DNA. The respective conformational geometry for RNA and DNA duplexes was determined from X ray diffraction analysis of nucleic acid fibers (Arnott and Hukins, Biochem. Biophys. Res. Comm., 1970, 47, 1504.) In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger, W. (1984) Principles of Nucleic Acid Structure, Springer-Verlag, New York, N.Y.). As used herein, B-form geometry is inclusive of both C2′-endo pucker and O4′-endo pucker. This is consistent with Berger, et. al., Nucleic Acids Research, 1998, 26, 2473-2480, who pointed out that in considering the furanose conformations which give rise to B-form duplexes consideration should also be given to a O4′-endo pucker contribution.

DNA:RNA hybrid duplexes, however, are usually less stable than pure RNA:RNA duplexes, and depending on their sequence may be either more or less stable than DNA:DNA duplexes (Searle et al., Nucleic Acids Res., 1993, 21, 2051-2056). The structure of a hybrid duplex is intermediate between A- and B-form geometries, which may result in poor stacking interactions (Lane et al., Eur. J. Biochem., 1993, 215, 297-306; Fedoroff et al., J. Mol. Biol., 1993, 233, 509-523; Gonzalez et al., Biochemistry, 1995, 34, 4969-4982; Horton et al., J. Mol. Biol., 1996, 264, 521-533). The stability of the duplex formed between a target RNA and a synthetic sequence is central to therapies such as but not limited to antisense and RNA interference as these mechanisms require the binding of a synthetic oligomer strand to an RNA target strand. In the case of antisense, effective inhibition of the mRNA requires that the antisense DNA have a very high binding affinity with the mRNA. Otherwise the desired interaction between the synthetic oligomer strand and target mRNA strand will occur infrequently, resulting in decreased efficacy.

One routinely used method of modifying the sugar puckering is the substitution of the sugar at the 2′-position with a substituent group that influences the sugar geometry. The influence on ring conformation is dependant on the nature of the substituent at the 2′ position. A number of different substituents have been studied to determine their sugar puckering effect. For example, 2′-halogens have been studied showing that the 2′ fluoro derivative exhibits the largest population (65%) of the C3′ endo form, and the 2′ iodo exhibits the lowest population (7%). The populations of adenosine (2′ OH) versus deoxyadenosine (2′ H) are 36% and 19%, respectively. Furthermore, the effect of the 2′ fluoro group of adenosine dimers (2′ deoxy 2′ fluoroadenosine 2′ deoxy 2′ fluoro-adenosine) is further correlated to the stabilization of the stacked conformation.

As expected, the relative duplex stability can be enhanced by replacement of 2′-OH groups with 2′-F groups thereby increasing the C3′-endo population. It is assumed that the highly polar nature of the 2° F. bond and the extreme preference for C3′ endo puckering may stabilize the stacked conformation in an A-form duplex. Data from UV hypochromicity, circular dichroism, and 1H NMR also indicate that the degree of stacking decreases as the electronegativity of the halo substituent decreases. Furthermore, steric bulk at the 2′ position of the sugar moiety is better accommodated in an A-form duplex than a B-form duplex. Thus, a 2′ substituent on the 3′ terminus of a dinucleoside monophosphate is thought to exert a number of effects on the stacking conformation: steric repulsion, furanose puckering preference, electrostatic repulsion, hydrophobic attraction, and hydrogen bonding capabilities. These substituent effects are thought to be determined by the molecular size, electronegativity, and hydrophobicity of the substituent. Melting temperatures of complementary strands is also increased with the 2′ substituted adenosine diphosphates. It is not clear whether the 3′ endo preference of the conformation or the presence of the substituent is responsible for the increased binding. However, greater overlap of adjacent bases (stacking) can be achieved with the 3′ endo conformation.

One synthetic 2′-modification that imparts increased nuclease resistance and a very high binding affinity to nucleotides is the 2-methoxyethoxy (2′-MOE, 2′-OCH₂CH₂OCH₃) side chain (Baker et al., J. Biol. Chem., 1997, 272, 11944-12000). One of the immediate advantages of the 2′-MOE substitution is the improvement in binding affinity, which is greater than many similar 2′ modifications such as O-methyl, O-propyl, and O-aminopropyl. Oligomers having the 2′-O-methoxyethyl substituent also have been shown to be antisense inhibitors of gene expression with promising features for in vivo use (Martin, P., Helv. Chim. Acta, 1995, 78, 486-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926). Relative to DNA, the oligomers having the 2′-MOE modification displayed improved RNA affinity and higher nuclease resistance. Chimeric oligomers having 2′-MOE substituents in the wing nucleosides and an internal region of deoxy-phosphorothioate nucleotides (also termed a gapped oligomer or gapmer) have shown effective reduction in the growth of tumors in animal models at low doses. 2′-MOE substituted oligomers have also shown outstanding promise as antisense compounds in several disease states. One such MOE substituted oligomer is presently being investigated in clinical trials for the treatment of CMV retinitis.

To better understand the higher RNA affinity of 2′-O-methoxyethyl substituted RNA and to examine the conformational properties of the 2′-O-methoxyethyl substituent, two dodecamer oligonucleotides were synthesized having SEQ D NO: 1 (CGCGAAUUCGCG) and SEQ ID NO: 2 (GCGCUUAAGCGC). These self-complementary strands have every 2′-position modified with a 2′-O-methoxyethyl. The duplex was crystallized at a resolution of 1.7 Ångstrom and the crystal structure was determined. The conditions used for the crystallization were 2 mM oligonucleotide, 50 mM Na Hepes pH 6.2-7.5, 10.50 mM MgCl2, 15% PEG 400. The crystal data showed: space group C2, cell constants a=41.2 Å, b=34.4 Å, c=46.6 Å, =92.4°. The resolution was 1.7 Å at −170° C. The current R=factor was 20% (Rfree 26%).

This crystal structure is believed to be the first crystal structure of a fully modified RNA oligonucleotide analogue. The duplex adopts an overall A-form conformation and all modified sugars display C3′-endo pucker. In most of the 2′-O-substituents, the torsion angle around the A′-B′ bond, as depicted in Structure II below, of the ethylene glycol linker has a gauche conformation. For 2′-MOE, A′ and B′ of Structure II below are methylene moieties of the ethyl portion of the MOE and R′ is the methoxy portion.

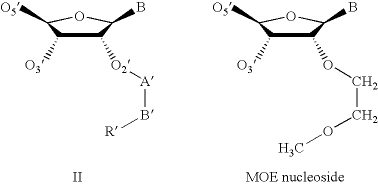

In the crystal, the 2′-MOE RNA duplex adopts a general orientation such that the crystallographic 2-fold rotation axis does not coincide with the molecular 2-fold rotation axis. The duplex adopts the expected A-type geometry and all of the 24 2′-MOE substituents were visible in the electron density maps at full resolution. The electron density maps as well as the temperature factors of substituent atoms indicate flexibility of the 2′-MOE substituent in some cases.

Most of the 2′-MOE substituents display a gauche conformation around the C—C bond of the ethyl linker. However, in two cases, a trans conformation around the C—C bond is observed. The lattice interactions in the crystal include packing of duplexes against each other via their minor grooves. Therefore, for some residues, the conformation of the 2′-O-substituent is affected by contacts to an adjacent duplex. In general, variations in the conformation of the substituents (e.g. g+ or g− around the C—C bonds) create a range of interactions between substituents, both inter-strand, across the minor groove, and intra-strand. At one location, atoms of substituents from two residues are in van der Waals contact across the minor groove. Similarly, a close contact occurs between atoms of substituents from two adjacent intra-strand residues.

Previously determined crystal structures of A-DNA duplexes were for those that incorporated isolated 2′-O-methyl T residues. In the crystal structure noted above for the 2′-MOE substituents, a conserved hydration pattern has been observed for the 2′-MOE residues. A single water molecule is seen located between O2′, O3′ and the methoxy oxygen atom of the substituent, forming contacts to all three of between 2.9 and 3.4 Å. In addition, oxygen atoms of substituents are involved in several other hydrogen bonding contacts. For example, the methoxy oxygen atom of a particular 2′-O-substituent forms a hydrogen bond to N3 of an adenosine from the opposite strand via a bridging water molecule.

In several cases a water molecule is trapped between the oxygen atoms O2′, O3′ and OC′ of modified nucleosides. 2′-MOE substituents with trans conformation around the C—C bond of the ethylene glycol linker are associated with close contacts between OC′ and N2 of a guanosine from the opposite strand, and, water-mediated, between OC′ and N3(G). When combined with the available thermodynamic data for duplexes containing 2′-MOE modified strands, this crystal structure allows for further detailed structure-stability analysis of other modifications.

In extending the crystallographic structure studies, molecular modeling experiments were performed to study further enhanced binding affinity of oligonucleotides having 2′-O-modifications. The computer simulations were conducted on compounds of SEQ ID NO: 1, above, having 2′-O-modifications located at each of the nucleosides of the oligonucleotide. The simulations were performed with the oligonucleotide in aqueous solution using the AMBER force field method (Cornell et al., J. Am. Chem. Soc., 1995, 117, 5179 5197) (modeling software package from UCSF, San Francisco, Calif.). The calculations were performed on an Indigo2 SGI machine (Silicon Graphics, Mountain View, Calif.).

Further 2′-O-modifications that will have a 3′-endo sugar influence include those having a ring structure that incorporates a two atom portion corresponding to the A′ and B′ atoms of Structure II. The ring structure is attached at the 2′ position of a sugar moiety of one or more nucleosides that are incorporated into an oligonucleotide. The 2′-oxygen of the nucleoside links to a carbon atom corresponding to the A′ atom of Structure II. These ring structures can be aliphatic, unsaturated aliphatic, aromatic or heterocyclic. A further atom of the ring (corresponding to the B′ atom of Structure II), bears a further oxygen atom, or a sulfur or nitrogen atom. This oxygen, sulfur or nitrogen atom is bonded to one or more hydrogen atoms, alkyl moieties, or haloalkyl moieties, or is part of a further chemical moiety such as a ureido, carbamate, amide or amidine moiety. The remainder of the ring structure restricts rotation about the bond joining these two ring atoms. This assists in positioning the “further oxygen, sulfur or nitrogen atom” (part of the R position as described above) such that the further atom can be located in close proximity to the 3′-oxygen atom (O3′) of the nucleoside.

Another suitable 2′-sugar substituent group that gives a 3′-endo sugar conformational geometry is the 2′ OMe group. 2′-Substitution of guanosine, cytidine, and uridine dinucleoside phosphates with the 2′-OMe group showed enhanced stacking effects with respect to the corresponding native (2′-OH) species leading to the conclusion that the sugar is adopting a C3′-endo conformation. In this case, it is believed that the hydrophobic attractive forces of the methyl group tend to overcome the destabilizing effects of its steric bulk.

The ability of oligonucleotides to bind to their complementary target strands is compared by determining the melting temperature (Tm) of the hybridization complex of the oligonucleotide and its complementary strand. The melting temperature (Tm), a characteristic physical property of double helices, denotes the temperature (in degrees centigrade) at which 50% helical (hybridized) versus coil (unhybridized) forms are present. Tm is measured by using the UV spectrum to determine the formation and breakdown (melting) of the hybridization complex. Base stacking, which occurs during hybridization, is accompanied by a reduction in UV absorption (hypochromicity). Consequently, a reduction in UV absorption indicates a higher Tm. The higher the Tm, the greater the strength of the bonds between the strands.

Freier and Altmann, Nucleic Acids Research, 1997, 25, 4429-4443, have previously published a study on the influence of structural modifications of oligonucleotides on the stability of their duplexes with target RNA. In this study, the authors reviewed a series of oligonucleotides containing more than 200 different modifications that had been synthesized and assessed for their hybridization affinity and Tm. Sugar modifications studied included substitutions on the 2′-position of the sugar, 3′-substitution, replacement of the 4′-oxygen, the use of bicyclic sugars, and four member ring replacements. Several nucleobase modifications were also studied including substitutions at the 5, or 6 position of thymine, modifications of pyrimidine heterocycle and modifications of the purine heterocycle. Modified internucleoside linkages were also studied including neutral, phosphorus and non-phosphorus containing internucleoside linkages.

Increasing the percentage of C3′-endo sugars in a modified oligonucleotide targeted to an RNA target strand should preorganize this strand for binding to RNA. Of the several sugar modifications that have been reported and studied in the literature, the incorporation of electronegative substituents such as 2′-fluoro or 2′-alkoxy shift the sugar conformation towards the 3′ endo (northern) pucker conformation. This preorganizes an oligonucleotide that incorporates such modifications to have an A-form conformational geometry. This A-form conformation results in increased binding affinity of the oligonucleotide to a target RNA strand.

In addition, for 2′-substituents containing an ethylene glycol motif, a gauche interaction between the oxygen atoms around the O—C—C—O torsion of the side chain may have a stabilizing effect on the duplex (Freier ibid.). Such gauche interactions have been observed experimentally for a number of years (Wolfe et al., Acc. Chem. Res., 1972, 5, 102; Abe et al., J. Am. Chem. Soc., 1976, 98, 468). This gauche effect may result in a configuration of the side chain that is favorable for duplex formation. The exact nature of this stabilizing configuration has not yet been explained. While we do not want to be bound by theory, it may be that holding the O—C—C—O torsion in a single gauche configuration, rather than a more random distribution seen in an alkyl side chain, provides an entropic advantage for duplex formation.

Representative 2′-substituent groups amenable to the present invention that give A-form conformational properties (3′-endo) to the resultant duplexes include 2′-O-alkyl, 2′-O-substituted alkyl and 2′-fluoro substituent groups. Options for the substituent groups are various alkyl and aryl ethers and thioethers, amines and monoalkyl and dialkyl substituted amines. It is further intended that multiple modifications can be made to one or more of the oligomeric compounds of the invention at multiple sites of one or more monomeric subunits (nucleosides are suitable) and or internucleoside linkages to enhance properties such as but not limited to activity in a selected application. Tables I through VII list nucleoside and internucleotide linkage modifications/replacements that have been shown to give a positive ΔTm per modification when the modification/replacement was made to a DNA strand that was hybridized to an RNA complement.

TABLE I
Modified DNA strand having 2′-substituent groups that gave an
overall increase in Tm against an RNA complement:
Positive ΔTm/mod

2′-substituents 2′-OH
2′-O—C₁—C₄ alkyl
2′-O—(CH₂)₂CH₃
2′-O—CH₂CH═CH₂
2′-F
2′-O—(CH₂)₂—O—CH₃
2′-(O—(CH₂)₂)₂—O—CH₃
2′-(O—(CH₂)₂)₃—O—CH₃
2′-(O—(CH₂)₂)₄—O—CH₃
2′-(O—(CH₂)₂)₃—O—(CH₂)₈CH₃
2′-O—(CH₂)₂CF₃
2′-O—(CH₂)₂OH
2′-O—(CH₂)₂F
2′-O—CH₂CH(CH₃)F
2′-O—CH₂CH(CH₂OH)OH
2′-O—CH₂CH(CH₂OCH₃)OCH₃
2′-O—CH₂CH(CH₃)OCH₃
2′-O—CH₂—C₁₄H₇O₂(—C₁₄H₇O₂ = Anthraquinone)
2′-O—(CH₂)₃—NH₂*
2′-O—(CH₂)₄—NH₂*
*These modifications can increase the Tm of oligonucleotides but can also decrease the Tm depending on positioning and number (motiff dependant).

TABLE II
Modified DNA strand having modified sugar ring (see structure x)
that gave an overall increase in Tm against an RNA complement:

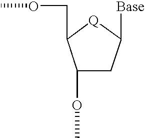

| | Positive ΔTm/mod |
|---|---|
| Q | —S— |
| | —CH₂— |

Note: In general ring oxygen substitution with sulfur or methylene had only a minor effect on Tm for the specific motiffs studied. Substitution at the 2′-position with groups shown to stabilize the duplex were destabilizing when CH₂ replaced the ring O. This is thought to be due to the necessary gauche interaction between the ring O with particular 2′-substituents (for example —O—CH₃ and —(O—CH₂CH₂)₃—O—CH₃.

TABLE III
Modified DNA strand having modified sugar ring that
give an overall increase in Tm against an RNA complement:

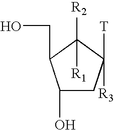

| | Positive ΔTm/mod |
|---|---|
| —C(H)R₁ effects | OH |
| (R₂, R₃ both = H) | CH₃* |
| | CH₂OH* |
| | OCH₃* |

*These modifications can increase the Tm of oligonucleotides but can also decrease the Tm depending on positioning and number (motiff dependant).

TABLE IV
Modified DNA strand having bicyclic substitute sugar modifications that
give an overall increase in Tm against an RNA complement:

| Formula | Positive ΔTm/mod |
|---|---|
| I | + |
| II | + |

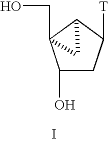

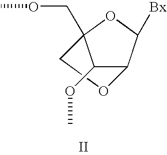

TABLE V
Modified DNA strand having modified heterocyclic base moieties that
give an overall increase in Tm against an RNA complement:

| Modification/Formula | Positive ΔTm/mod |
|---|---|
| Heterocyclic base | 2-thioT |
| modifications | 2′-O-methylpseudoU |
| | 7-halo-7-deaza purines |
| | 7-propyne-7-deaza purines |
| | 2-aminoA(2,6-diaminopurine) |
| | 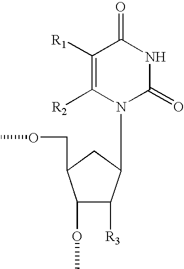 |
| (R₂, R₃ = H), R₁ = | Br |
| | \tilde{C}C—CH₃ |
| | (CH₂)₃NH₂ |
| | CH₃ |
| Motiffs-disubstitution | |
| R₁ = \tilde{C}C—CH₃, R₂ = H, R₃ = | F |
| R₁ = \tilde{C}C—CH₃, R₂ = H | R₃ = O—(CH₂)₂—O—CH₃ |
| R₁ = O—CH₃, R₂ = H, | R₃ = O—(CH₂)₂—O—CH₃* |

*This modification can increase the Tm of oligonucleotides but can also decrease the Tm depending on positioning and number (motiff dependant).

Substitution at R₁ can be stabilizing, substitution at R₂ is generally greatly destabilizing (unable to form anti conformation), motiffs with stabilizing 5 and 2′-substituent groups are generally additive e.g. increase stability.

Substitution of the O4 and O2 positions of 2′-O-methyl uridine was greatly duplex destabilizing as these modifications remove hydrogen binding sites that would be an expected result. 6-Aza T also showed extreme destabilization as this substitution reduces the pKa and shifts the nucleoside toward the enol tautomer resulting in reduced hydrogen bonding.

TABLE VI
DNA strand having at least one modified phosphorus containing
internucleoside linkage and the effect on
the Tm against an RNA complement:

| ΔTm/mod + | ΔTm/mod − |
|---|---|
| phosphoramidate (the 3′-bridging | phosphorothioate1 |
| atom replaced with an N(H)R | phosphoramidate1 |
| group, stabilization effect | methyl phosphonates1 |
| enhanced when also have 2′-F) | |

(1one of the non-bridging oxygen atoms replaced with S, N(H)R or —CH₃)

TABLE VII
DNA strand having at least one non-phosphorus containing internucleoside
linkage and the effect on the Tm against an RNA complement:
Positive ΔTm/mod

—CH₂C(═O)NHCH₂—*
—CH₂C(═O)N(CH₃)CH₂—*
—CH₂C(═O)N(CH₂CH₂CH₃)CH₂—*
—CH₂C(═O)N(H)CH₂—(motiff with 5′-propyne on T's)
—CH₂N(H)C(═O)CH₂—*
—CH₂N(CH₃)OCH₂—*
—CH₂N(CH₃)N(CH₃)CH₂—*
*This modification can increase the Tm of oligonucleotides but can also decrease the Tm depending on positioning and number (motiff dependant).

Notes: In general carbon chain internucleotide linkages were destabilizing to duplex formation. This destabilization was not as severe when double and tripple bonds were utilized. The use of glycol and flexible ether linkages were also destabilizing.

Suitable ring structures of the invention for inclusion as a 2′-O modification include cyclohexyl, cyclopentyl and phenyl rings as well as heterocyclic rings having spacial footprints similar to cyclohexyl, cyclopentyl and phenyl rings. Suitable 2′-O-substituent groups of the invention include, but are not limited to, 2′-O-(trans 2-methoxy cyclohexyl, 2′-O-(trans 2-methoxy cyclopentyl, 2′-O-(trans 2-ureido cyclohexyl) and 2′-O-(trans 2-methoxyphenyl).

Examples of some modified nucleosides that are expected to have 3′-endo sugar conformation are shown below in Table VIII. These examples are meant to be representative and not exhaustive.

TABLE VIII

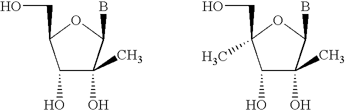

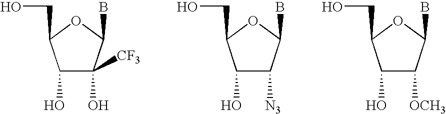

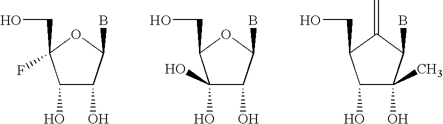

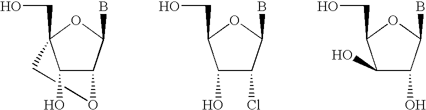

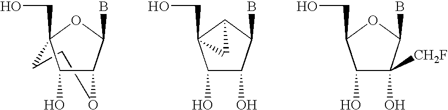

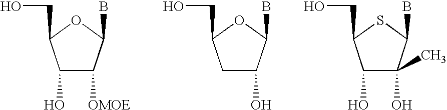

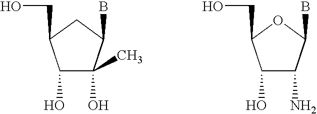

Although the overall stability of the DNA:RNA hybrids depends on several factors including sequence-dependencies and the purine content in the DNA or RNA strands DNA:RNA hybrids are usually less stable than RNA:RNA duplexes and, in some cases, even less stable than DNA:DNA duplexes. Available experimental data attributes the relatively lowered stability of DNA:RNA hybrids largely to its intermediate conformational nature between DNA:DNA (B-family) and RNA:RNA (A-family) duplexes. The overall thermodynamic stability of nucleic acid duplexes may originate from several factors including the conformation of backbone, base-pairing and stacking interactions. While it is difficult to ascertain the individual thermodynamic contributions to the overall stabilization of the duplex, it is reasonable to argue that the major factors that promote increased stability of hybrid duplexes are better stacking interactions (electrostatic π-π interactions) and more favorable groove dimensions for hydration. The C2′-S-methyl substitution has been shown to destabilize the hybrid duplex. The notable differences in the rise values among the three hybrids may offer some explanation. While the 2′-S-methyl group has a strong influence on decreasing the base-stacking through high rise values (˜3.2 Å), the 2′-O-methyl group makes the overall structure more compact with a rise value that is equal to that of A-form duplexes (˜2.6 Å). Despite its overall A-like structural features, the SMe_DNA:RNA hybrid structure possesses an average rise value of 3.2 Å which is quite close to that of B-family duplexes. In fact, some local base-steps (CG steps) may be observed to have unusually high rise values (as high as 4.5 Å). Thus, the greater destabilization of 2′-S-methyl substituted DNA:RNA hybrids may be partly attributed to poor stacking interactions.

In one aspect of the present invention oligomeric compounds are affinity modified by modification of one or more of their internucleoside linkages. One suitable internucleoside linkage known to result in reduced affinity and increased nuclease resistance is the phosphorothiate internucleoside linkage. One suitable group of oligonucleotides having modified internucleotide linkages for use in the present invention include oligonucleotides having alternating phosphodiester and phosphorothioate internucleotide linkages optionally including a 5′-phosphate group. Another suitable group includes oligoribonucleotides having alternating phosphodiester and phosphorothioate internucleotide linkages optionally including a 5′-phosphate group.

In another aspect of the present invention oligomeric comounds are affinity modified by modification or substitution of the furanose sugar moiety. Suitable 2′-substituent groups amenable to the present invention include but are not limited to 2′-O-methyl, 2′-F, 2′-methoxyethoxy (—O—CH2-CH2-O—CH3) and 2′-amino. Suitable internucleotide linkages for these oligonucleotides include full phosphodiester and alternating phosphodiester and phosphorothioate.

In a further aspect of the present invention the heterocyclic bases of selected nucleosides are modified to effect the desired affinity modification of a resulting oligomeric compound. One group of suitable modified heterocyclic bases amenable to the present invention include N3-methyl uridine, inosine, 2,6-diaminopurine, purine and 2-aminopurine.

Oligonucleotides having a plurality of identical or different modifications are also amenable to the present invention. For the purpose of the present invention oligonucleotides are described as having at least 2 different modifications when at least two modifications are present that are not naturally occurring. These modifications can be associated with a single nucleoside or can be associated with a plurality of nucleosides within an oligonucleotide. More frequently the modifications are spread out to achieve a desired result such as affinity modulation over a selected region or regions.

Some oligonucleotides having at least two different types of modifications include an oligonucleotide having at least one 2′-F modified sugar moiety or one 2′-O-methyl modified sugar moiety in combination with a phosphate group at the 5′-terminus having phosphodiester, phosphorothioate or a mixture of both phosphodiester and phosphorothioate internucleotide linkages; an oligonucleotide having at least one 2′-F modified and at least one 2′-O-methyl modified sugar moieties having phosphodiester, phosphorothioate or a mixture of both phosphodiester and phosphorothioate internucleotide linkages and optionally having a 5′-phosphate group; and an oligonucleotide having at least one inosine heterocyclic base moiety and at least one diaminopurine heterocyclic base moiety having phosphodiester, phosphorothioate or a mixture of both phosphodiester and phosphorothioate internucleotide linkages and optionally having a 5′-phosphate group.

Representative examples of oligoribonucleotides having alternating P═O/P═S backbones indlude:

| | | |
|---|---|---|
| SEQ ID NO: | ISIS No: | Sequence |

| | | |
|---|---|---|
| 5 | 263186 | C.U*G.C*U.A*G.C*C.U*C.U*G.G*A.U*U.U*G.dT*dT |
| 6 | 263188 | C.U*U.C*U.G*G.C*A.U*C.C*G.G*U.U*U.A*G.dT*dT |
| 7 | 263187 (S) | C.A*A.A*U.C*C.A*G.A*G.G*C.U*A.G*C.A*G.dT*dT |
| 8 | 263187 (S) | C.A*A.A*U.C*C.A*G.A*G.G*C.U*A.G*C.A*G.dT*dT |
| 9 | 263189 (S) | C.U*A.A*A.C*C.G*G.A*U.G*C.C*A.G*A.A*G.dT*dT |

Representative examples of oligoribonucleotides having either P═O or P═S internucleotide linkages and further having a 5′-phosphate group include:

| | | |
|---|---|---|
| SEQ ID NO: | ISIS No: | Sequence |

| | | |
|---|---|---|
| 10 | 300851 | 5′-P C.U.G.C.U.A.G.C.C.U.C.U.G.G.A.U.U.U.G.A |
| 11 | 308745 | 5′-P U*U*U*A*U*C*G*C*U*U*C*U*C*G*U*U*G*C*U*U |
| 12 | 300857 | 5′-P-*G*C*U*A*G*C*C*U*C*U*G*G*A*U*U*U*G*A |
| 13 | 303912 | 5′-P U*U*U*G*U*C*U*C*U*G*G*U*C*C*U*U* A*C*U*U |
| 14 | 303914 | 5′-P C*C*U*U*U*U*U*G*U*C*U*C*U*G*G*U*C*C*U*U |
| 15 | 303916 | 5′-P U*C*U*C*U*G*G*U*C*C*U*U*A*C*U*U*C*C*C*C |

Representative examples of oligoribonucleotides having 2′-O-methyl sugar modifications at selected positions and having phosphodiester internucleoside linkages include:

| | | |
|---|---|---|
| SEQ ID NO: | ISIS No: | Sequence |

| | | |
|---|---|---|
| 16 | 271067 | C.U.G.C.U.A.G.C.C.U.C.U.G.G.mA.mU.mU.mU.mG.mU.mU |
| 17 | 271065 | C.U.G.C.U.A.G.mC.mC.mU.mC.mU.G.G.A.U.U.U.G.dT.dT |
| 18 | 271071 | mC.mU.mG.mC.mU.mA.G.C.C.U.C.U.G.G.A.U.U.U.G.dT.dT |
| 19 | 271076 | mC.mU.G.mC.mU.A.G.C.C.U.C.U.G.G.A.U.U.U.G.dT.dT |
| 20 | 271072 | C.mU.mG.mC.mU.mA.G.C.C.U.C.U.G.G.A.U.U.U.G.dT.dT |
| 21 | 271073 | C.U.mG.mC.mU.mA.mG.C.C.U.C.U.G.G.A.U.U.U.G.dT.dT |
| 22 | 271074 | C.U.G.mC.mU.mA.mG.mC.C.U.C.U.G.G.A.U.U.U.G.dT.dT |
| 23 | 271075 | C.U.G.C.mU.mA.mG.mC.mC.U.C.U.G.G.A.U.U.U.G.dT.dT |
| 24 | 271081 | C.U.G.C.U.A.G.mC.mC.mU.mC.mU.mG.mG.mA.mU.mU |
| | | mU.mG.mU.mU |
| 25 | 271080 (S) | C.A.A.A.U.C.C.mA.mG.mA.mG.mG.mC.mU.mA.mG.mC.mA. |
| | | mG.mU.mU |
| 26 | 271070 (S) | C.mA.A.mA.U.mC.C.mA.G.mA.G.mG.C.mU.A.mG.C.mA. |
| | | G.mU.dT |
| 27 | 271079 | C.U.G.C.U.A.G.C.C.U.C.U.mG.mG.mA.mU.mU.mU.mG.mU.mU |
| 28 | 271078 | C.U.G.C.U.A.G.mC.mC.U.C.mU.G.G.A.U.U.U.G.dT.dT |
| 29 | 271077 | C.U.G.C.U.A.G.C.C.mU.mC.U.G.G.A.U.U.U.G.dT.dT |
| 30 | 271069 | C.mU.G.mC.U.mA.G.mC.C.mU.C.mU.G.mG.A.mU.U.mU. |
| | | G.mU.dT |
| 31 | 283547 | mC.mU.G.C.U.A.G.C.C.U.C.U.G.G.A.U.U.U.G.dT.dT |
| 32 | 293999 | C.mU.G.C.U.A.G.C.C.U.C.U.G.G.A.U.U.U.G.dT.dT |
| 33 | 294000 | mC.U.G.C.U.A.G.C.C.U.C.U.G.G.A.U.U.U.G.dT.d |
| 34 | 290223 | mC.mU.mG.mC.mU.mA.mG.mC.mC.mU.mC.mU.mG.mG.mA. |
| | | mU.mU.mU.mG.dT.dT |
| 35 | 300852 | C.U.G.mC.mU.mA.mG.C.C.U.C.U.G.G.A.U.mU.mU.mG.mA |
| 36 | 300854 | mC.mU.mA.mG.C.C.U.C.U.G.G.A.U.mU.mU.mG.mA |
| 37 | 271068 (S) | C.A.A.A.U.C.C.A.G.A.G.G.C.U.mA.mG.mC.mA.mG.mU.mU |
| 38 | 271066 (S) | C.A.A.A.U.C.C.mA.mG.mA.mG.mG.C.U.A.G.C.A.G.dT.dT |
| 39 | 290224 (S) | mC.mA.mA.mA.mU.mC.mC.mA.mG.mA.mG.mG.mC.mU.mA. |
| | | mG.mC.mA.mG.dT.dT |

Representative examples of oligoribonucleotides having 2′-O-methyl sugar modifications at selected positions and having mixed phosphodiester and phosphorothioate internucleoside linkages include:

| | | |
|---|---|---|
| SEQ ID NO: | ISIS No: | Sequence |

| | | |
|---|---|---|
| 40 | 300856 | mC*mU*mA*mG.C.C.U.C.U.G.G.A.U.mU*mU*mG*mA |
| 41 | 300858 | C*U*G*mC*mU*mA*mG*C*C*U*C*U*G*G*A*U*mU*mU* |
| | | mG*mA |
| 42 | 300860 | mC*mU*mA*mG*C*C*U*C*U*G*G*A*U*mU*mU*mG*mA |
| 43 | 303913 | mG*mU*mC*mU*C*U*G*G*U*C*C*U*U*mA*mC*mU*mU |
| 44 | 303915 | mU*mU*mU*mU*G*U*C*U*C*U*G*G*U*mC*mC*mU*mU |
| 45 | 303917 | mC*mU*mG*mG*U*C*C*U*U*A*C*U*U*mC*mC*mC*mC |
| 46 | 271083 | C.U.G.C.U.A.G.C.C.U.C.U.G.G.A.U.mU*mU*mG*mU*mU |
| 47 | 271082 | C.U.G.C.U.A.G.C.C.U.C.U.G.G.A.U.mU*mU*mG*mA*mC |

Representative examples of oligoribonucleotides having 2′-F sugar modifications at selected positions and having phosphodiester internucleoside linkages include:

| | | |
|---|---|---|
| SEQ ID NO: | ISIS No: | Sequence |

| | | |
|---|---|---|
| 48 | 271061 | C.U.G.C.U.A.G.C.C.U.C.U.G.G.A.U.U.U.G.2′-F-U.dT |
| 49 | 271059 | C.U.G.C.U.A.G.C.C.U.C.U.G.G.A.2′-F-U.2′-F-U.2′-F-U.G.dT.dT |
| 50 | 271060 | 2′-F-C.2′-F-U.G.2′F-C.2′-F-.A.G.C.C.U.C.U.G.G.A.U.U.U.G. |
| | | dT.dT |
| 51 | 271058 | C.U.G.C.U.A.G.2′-F-C.2′-F-C.2′-F-U.2′-F-C.2′-F-U.G.G.A.U.U. |
| | | U.G.dT.dT |
| 52 | 296894 | 2′-F-U.2′-F-U.G.2′-F-U.2′-F-U.2′-F-C.2′-F-C.2′-F-U.A.U.A.A.2′-F- |
| | | C.2′-F-U.G.G.2′-F-U.A.A.dT.dT |
| 53 | 279471 (ND) | 2′-F-C.2′-F-U.2′-F-G.2′-F-C.2′-F-U.2′-F-A.2′-F-G.2′-F-C.2′F-C.2′- |
| | | F-U.2′-F-C.2′-F-U.2′-F-G.2′-F-G.2′-F-A.2′F-U.2′F-U.2′-F-U.2′-F- |
| | | G.dT.dT |
| 54 | 279467 (SND) | 2′-F-C.2′-F-A.2′-F-A.2′-F-A.2′-F-U.2′-F-C.2′-F-C.2′-F-A.2′-F-G.2′- |
| | | F-A.2′-F-G.2′-F-G.2′-F-C.2′-F-U.2′-F-A.2′-F-G.2′-F-C.2′-F-A.2′-F- |
| | | G.dT.dT |

Representative examples of oligoribonucleotides having 2′-amino sugar modifications at selected positions and having phosphodiester internucleoside linkages include:

| | | |
|---|---|---|
| SEQ ID NO: | ISIS No: | Sequence |

| | | |
|---|---|---|
| 55 | 271392 | C.U.G.C.U.A.G.C.C.2′-N-U.C.2′-N.U.G.G.A.U.U.U.G.dT.dT |
| 56 | 271393 | C.2′-N-U.G.C.2′-N-U.A.G.C.C.U.C.U.G.G.A.2′-N-U.U.2′-N- |
| | | U.G.dT.dT |

Representative examples of oligoribonucleotides having at least one modified heterocyclic base moiety include:

N3 methyl uridine modified oligoribonucleotides

| | | |
|---|---|---|
| SEQ ID NO: | ISIS No: | Sequence |

| | | |
|---|---|---|
| 57 | 271390 | C.U.G.C.U.A.G.C.C.3-M-U.C.3.M-U.G.G.A.U.U.U.G.dT.dT |
| 58 | 271391 | C.3-M-U.G.C.3-M-U.A.G.C.C.U.C.U.G.G.A.3-M.U.U.3-M- |
| | | U.G.dT.dT |
| 59 | 271389 (S) | C.A.A.A.3-M-U.C.C.A.G.A.G.G.C.3-M-U.A.G.C.A.G.dT.dT |

Inosine modified oligoribonucleotides

| | | |
|---|---|---|
| SEQ ID NO: | ISIS No: | Sequence |

| | | |
|---|---|---|
| 60 | 271388 | C.U.I.C.U.A.I.C.C.U.C.U.I.I.A.U.U.U.I.dT.dT |
| 61 | 293994 | C.U.G.C.U.A.G.C.C.U.C.U.I.I.A.U.U.U.G.dT.dT |
| 62 | 293995 | C.U.I.C.U.A.I.C.C.U.C.U.G.G.A.U.U.U.G.dT.dT |
| 63 | 293996 | C.U.G.C.U.A.G.C.C.U.C.U.G.G.A.U.U.U.I.dT.dT |
| 64 | 293997 | C.U.I.C.U.A.I.C.C.U.C.U.I.I.A.U.U.U.G.dT.dT |
| 65 | 271387 (S) | C.A.A.A.U.C.C.A.I.A.I.I.C.U.A.I.C.A.I.dT.dT |
| 66 | 296893 (S) | I.C.A.C.A.U.C.C.A.I.G.I.A.C.C.C.G.I.G.dT.dT |
| 67 | 296892 (S) | C.C.U.I.U.G.A.I.C.A.I.C.C.G.C.I.G.I.G.dT.dT |

2,6-Diaminopurine modified oligoribonucleotides

| | | |
|---|---|---|
| SEQ ID NO: | ISIS No: | Sequence |

| | | |
|---|---|---|
| 68 | 271386 | C.U.G.C.U.DAP.G.C.C.U.C.U.G.G.DAP.U.U.U.G.dT.dT |
| 69 | 271385 (S) | C.DAP.A.DAP.U.C.C.DAP.G.DAP.G.G.C.U.DAP.G.C.DAP.G. |
| | | dT.dT |

2-Aminopurine modified oligoribonucleotides

| | | |
|---|---|---|
| SEQ ID NO: | ISIS No: | Sequence |

| | | |
|---|---|---|
| 70 | 271384 | C.U.G.C.U.2AP.G.C.C.U.C.U.G.G.2AP.U.U.U.G.dT.dT |
| 71 | 271383 (S) | C.2AP.A.2AP.U.C.C.2AP.G.2AP.G.G.C.U.2AP.G.C.2AP. |
| | | G.dT.dT |

Purine modified oligoribonucleotides

| | | |
|---|---|---|
| SEQ ID NO: | ISIS No: | Sequence |

| | | |
|---|---|---|
| 72 | 271382 | C.U.G.C.U.Pu.G.C.C.U.C.U.G.G.Pu.U.U.U.G.dT.dT |
| 73 | 271381 (S) | C.Pu.A.Pu.U.C.C.Pu.G.Pu.G.G.C.U.Pu.G.C.Pu.G.dT.dT |

Representative examples of oligoribonucleotides having at least two different chemistry modifications include:

2′-F, with 5′-phosphate, P═O/P═S mixed backbone

| | | |
|---|---|---|
| SEQ ID NO: | ISIS No: | Sequence |

| | | |
|---|---|---|
| 74 | 317466 | 5′P.2′-F-U*2′-F-U*2′-F-U*G*U*C*U*C*U*G*G*U*C*C*U*U* |
| | | A*2′-F-C*2′-F-U*U |
| 75 | 317468 | 5′-P.2′-F-U.2′-F-U.2′-F-U.G.U.C.U.C.U.G.G.U.C.C.U.U.A.2′-F- |
| | | C.2′-F-U.U |
| 76 | 317502 | 5′P.2′-F-U*2′-F-U*2′-F-U*G*2′-F-U*2′-F-C*2′-F-U*C*U*G* |
| | | G*U*C*2′-F-C*2′-F-U*2′-F-U*A*2′-F-C*2′-F-U*U |
| 77 | 319018 | 5′-P 2′-F-U.2′-F-U.2′-F-U.2′-F-G.2′F-U.2′-F-C.2′-F-U.2′-F-C.2′-F- |
| | | U.2′-F-G.2′-F-G.2′-F-U.2′-F-C.2′-F-C.2′-F-U.2′-F-U.2′-F-A.2′-F- |
| | | C.2′-F-U.2′-F-U |
| 78 | 319022 | 5′-P 2′-F-U*2′F-U*2′-F-G*2′-F-U*2′-F-C*2′-F-U*2′-F-C |
| | | *2′-F-U*2′-F-G*2′-F-G*2′-F-U*2′-F-C*2′-F-C*2′-F-U*2′F-U*2′- |
| | | F-A*2′-F-C*2′-F-U*2′-F-U |
| 79 | 319019 (S) | 5′-P 2′-F-A.2′-F-A.2′-F-G.2′-F-U.2′-F-A.2′-F-A.2′-F-G.2′-F-G.2′-F- |
| | | A.2′-F-C.2′-F-C.2′-F-A.2′-F-G.2′-F-A.2′-F-G.2′-F-A.2′-F-C.2′-F- |
| | | A.2′-F-A.2′-F-A |

2′-F and 2′-O-methyl

| | | |
|---|---|---|
| SEQ ID NO: | ISIS No: | Sequence |

| | | |
|---|---|---|
| 80 | 283546 | 2′-F-C.2′-F-U.mG.2′-F-C.2′-F-U.mA.mG.2′-F-C.2′-F-C.2′-F-U.2′- |
| | | F-C.2′-F-U.mG.mG.mA.2′-F-U.2′-F-U.2′F-U.mG.2′-F-U.dT |

2′-O-methyl and 5′-phosphate

| | | |
|---|---|---|
| SEQ ID NO: | ISIS No: | Sequence |

| | | |
|---|---|---|
| 81 | 271084 | 5′-P mC.mU.mG.mC.mU.A.G.C.C.U.C.U.G.G.A.U.U.U.G.dT.dT |
| 82 | 300853 | 5′-P C.U.G.mC.mU.mA.mG.C.C.U.C.U.G.G.A.U.mU.mU.mG.mA |
| 83 | 316449 | 5′-P.U*U*U*G*U*C*U*C*U*G*G*U*C*C*U*U*A*mC*mU* |
| | | mU |

2′-O-methyl and 5′-phosphate

| | | |
|---|---|---|
| SEQ ID NO: | ISIS No: | Sequence |

| | | |
|---|---|---|
| 84 | 300855 | 5′-P C*U*G*mC*mU*mA*mG.C.C.U.C.U.G.G.A.U.mU*mU* |
| | | mG*mA |
| 85 | 300859 | 5′-P C*U*G*mC*mU*mA*mG*C*C*U*C*U*G*G*A*U*mU* |
| | | mU*mG*mA |
| 86 | 308743 | 5′-P mU*mU*mU*G*U*C*U*C*U*G*G*U*C*C*U*U*A*mC* |
| | | mU*mU |
| 87 | 308744 | 5′-P mU*mC*mU*mC*mU*G*G*U*C*C*U*U*A*C*U*mU* |
| | | mC*mC*mC*mC |
| 88 | 319013 | 5′-P.U*U*U*G*U*C*U*C*U*G*G*U*C*C*U*mU*mA*mC* |
| | | mU*mU |
| 89 | 319014 | 5′-P.U*U*U*G*U*C*U*C*U*G*G*U*C*mC*mU*mU*mA* |
| | | mC*mU*mU |
| 90 | 319015 | 5′-P.U*U*U*G*U*C*U*C*U*G*G*mU*mC*mC*mU*mU* |
| | | mA*mC*mU*mU |
| 91 | 319016 | 5′-P.U*dT*dT*dG*dT*dC*dT*dC*U*G*G*U*C*C*U*U*A* |
| | | mC*mU*mU |
| 92 | 319017 | 5′-P.U*U*U*G*U*C*U*C*U*G*dG*dT*dC*dC*dT*dT*dA* |
| | | mC*mU*mU |

Inosine and diaminopurine

| SEQ ID NO: | ISIS No: | Sequence |
|---|---|---|
| 93 | 293998 | C.U.I.C.U.DAP.I.C.C.U.C.U.I.I.DAP.U.U.U.G.dT.dTd |

Representative examples of oligoribonucleotides having conjugate groups include:

| | | |
|---|---|---|
| SEQ ID NO: | ISIS No: | Sequence |

| | | |
|---|---|---|
| 94 | 271064 | C.U.G.C.U.A.G.C.C.U.C.U.G.G.A.U.U.U.G*dT*dT*3′-Bi |
| 95 | 27106 | Bi.18s.18s.18s.C.U.G.C.U.A.G.C.C.U.C.U.G.G.A.U.U.U.G.dT.dT |
| 96 | 271062 (S) | Bi.C.A.A.A.U.C.C.A.G.A.G.G.C.U.A.G.C.A.G.dT.dT |

Representative examples of oligoribonucleotides having 18S peg linkers include:

| | | |
|---|---|---|
| SEQ ID NO: | ISIS No: | Sequence |

| | | |
|---|---|---|
| 97 | 317469 | C.A.G.A.G.A.C.A.A.A.18S.U.U.U.G.U.C.U.C.U.G.G.U.C.C.U.U. |
| | | A.C.U.U.18S.A.A.G.U.A.A.G.G.A.C |
| 98 | 317470 | C.A.G.A.G.A.C.A.A.A.18S.U.U.U.G.U.C.U.C.U.G.G.U.C.C.U.U. |
| | | A.C.U.U |

18S peg linker and 5′-phosphate modified oligoribonucleotides

| | | |
|---|---|---|
| SEQ ID NO: | ISIS No: | Sequence |

| | | |
|---|---|---|
| 99 | 317471 | 5′-P.U.U.U.G.U.C.U.C.U.G.G.U.C.C.U.U.A.C.U.U.18S.A.A.G.U. |
| | | A.A.G.G.A.C |

Representative nucleosides having modified heterocyclic bases 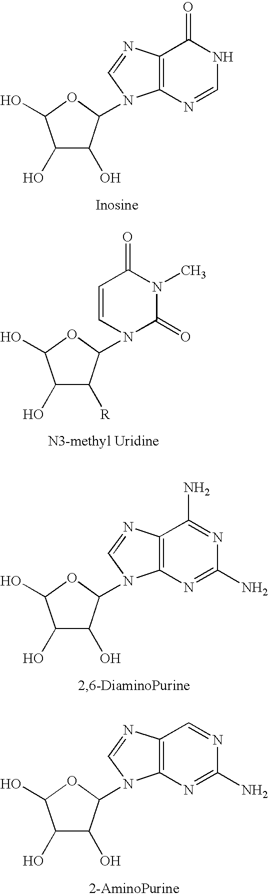
period—diester
asterick—Phosphorothioate
m—2′-O-methyl
3-M-U—N3-CH3-U
2′-N-U—2′-amino
I—Inosine
DAP—2,6-diaminopurine
2AP—2 amino purine
Pu—purine
Bi—Biotin conjugate
18s—18 atom peg spacer
Sequences are antisense and prepared by Dharmacon unless otherwise labeled
(S)=Sense strand
(ND)=Non-Dharmacon synthesized sequence (prepared in house)
(SND)=Sense non-Dharmacon

Compositions of the present invention will be useful for the modulation of gene expression. In one aspect of the present invention a targeted cell, group of cells, a tissue or an animal is contacted with a composition of the invention to effect reduction of message that can directly inhibit gene expression. In another embodiment the reduction of message indirectly upregulates a non-targeted gene through a pathway that relates the targeted gene to a non-targeted gene. Methods and models for the regulation of genes using oligomeric compounds of the invention are illustrated in the examples.

In another aspect a method of inhibiting gene expression is disclosed comprising contacting one or more cells, a tissue or an animal with a composition of the invention. Numerous procedures of how to use the compositions of the present invention are illustrated in the examples section.

Compositions of the invention modulate gene expression by hybridizing to a nucleic acid target resulting in loss of its normal function. As used herein, the term “target nucleic acid” or “nucleic acid target” is used for convenience to encompass any nucleic acid capable of being targeted including without limitation DNA, RNA (including pre-mRNA and mRNA or portions thereof) transcribed from such DNA, and also cDNA derived from such RNA. In one embodiment of the invention the target nucleic acid is a messenger RNA. In another embodiment the degradation of the targeted messenger RNA is facilitated by a RISC complex that is formed with oligomeric compounds of the invention. In another embodiment the degradation of the targeted messenger RNA is facilitated by a nuclease such as RNaseH.

The hybridization of an oligomeric compound of this invention with its target nucleic acid is generally referred to as “antisense”. Consequently, one mechanism in the practice of some embodiments of the invention is referred to herein as “antisense inhibition.” Such antisense inhibition is typically based upon hydrogen bonding-based hybridization of oligonucleotide strands or segments such that at least one strand or segment is cleaved, degraded, or otherwise rendered inoperable. In this regard, it is presently suitable to target specific nucleic acid molecules and their functions for such antisense inhibition.

The functions of DNA to be interfered with can include replication and transcription. Replication and transcription, for example, can be from an endogenous cellular template, a vector, a plasmid construct or otherwise. The functions of RNA to be interfered with can include functions such as translocation of the RNA to a site of protein translation, translocation of the RNA to sites within the cell which are distant from the site of RNA synthesis, translation of protein from the RNA, splicing of the RNA to yield one or more RNA species, and catalytic activity or complex formation involving the RNA which may be engaged in or facilitated by the RNA. In the context of the present invention, “modulation” and “modulation of expression” mean either an increase (stimulation) or a decrease (inhibition) in the amount or levels of a nucleic acid molecule encoding the gene, e.g., DNA or RNA. Inhibition is often the desired form of modulation of expression and mRNA is often a suitable target nucleic acid.

The compositions and methods of the present invention are also useful in the study, characterization, validation and modulation of small non-coding RNAs. These include, but are not limited to, microRNAs (miRNA), small nuclear RNAs (snRNA), small nucleolar RNAs (snoRNA), small temporal RNAs (stRNA) and tiny non-coding RNAs (tncRNA) or their precursors or processed transcripts or their association with other cellular components.

Small non-coding RNAs have been shown to function in various developmental and regulatory pathways in a wide range of organisms, including plants, nematodes and mammals. MicroRNAs are small non-coding RNAs that are processed from larger precursors by enzymatic cleavage and inhibit translation of mRNAs. stRNAs, while processed from precursors much like miRNAs, have been shown to be involved in developmental timing regulation. Other non-coding small RNAs are involved in events as diverse as cellular splicing of transcripts, translation, transport, and chromosome organization.

As modulators of small non-coding RNA function, the compositions of the present invention find utility in the control and manipulation of cellular functions or processes such as regulation of splicing, chromosome packaging or methylation, control of developmental timing events, increase or decrease of target RNA expression levels depending on the timing of delivery into the specific biological pathway and translational or transcriptional control. In addition, the compositions of the present invention can be modified in order to optimize their effects in certain cellular compartments, such as the cytoplasm, nucleus, nucleolus or mitochondria.

The compositions of the present invention can further be used to identify components of regulatory pathways of RNA processing or metabolism as well as in screening assays or devices.

Chemistries Defined

Unless otherwise defined herein, alkyl means C₁-C₁₂, C₁-C₈, or C₁-C₆, straight or (where possible) branched chain aliphatic hydrocarbyl.

Unless otherwise defined herein, heteroalkyl means C₁-C₁₂, C₁-C₈, or C₁-C₆, straight or (where possible) branched chain aliphatic hydrocarbyl containing at least one, and about 1 to about 3 hetero atoms in the chain, including the terminal portion of the chain. Suitable heteroatoms include N, O and S.

Unless otherwise defined herein, cycloalkyl means C₃-C₁₂, C₃-C₈, or C₃-C₆, aliphatic hydrocarbyl ring.

Unless otherwise defined herein, alkenyl means C₂-C₁₂, C₂-C₈, or C₂-C₆ alkenyl, which may be straight or (where possible) branched hydrocarbyl moiety, which contains at least one carbon-carbon double bond.

Unless otherwise defined herein, alkynyl means C₂-C₁₂, C₂-C₈, or C₂-C₆ alkynyl, which may be straight or (where possible) branched hydrocarbyl moiety, which contains at least one carbon-carbon triple bond.

Unless otherwise defined herein, heterocycloalkyl means a ring moiety containing at least three ring members, at least one of which is carbon, and of which 1, 2 or three ring members are other than carbon. The number of carbon atoms can vary from 1 to about 12, or from 1 to about 6, and the total number of ring members can vary from three to about 15, or from about 3 to about 8. Suitable ring heteroatoms are N, O and S. Suitable heterocycloalkyl groups include, but are not limited to, morpholino, thiomorpholino, piperidinyl, piperazinyl, homopiperidinyl, homopiperazinyl, homomorpholino, homothiomorpholino, pyrrolodinyl, tetrahydrooxazolyl, tetrahydroimidazolyl, tetrahydrothiazolyl, tetrahydroisoxazolyl, tetrahydropyrrazolyl, furanyl, pyranyl, and tetrahydroisothiazolyl.

Unless otherwise defined herein, aryl means any hydrocarbon ring structure containing at least one aryl ring. Suitable aryl rings have about 6 to about 20 ring carbons. Additional aryl rings include phenyl, napthyl, anthracenyl, and phenanthrenyl.

Unless otherwise defined herein, hetaryl means a ring moiety containing at least one fully unsaturated ring, the ring consisting of carbon and non-carbon atoms. The ring system can contain about 1 to about 4 rings. The number of carbon atoms can vary from 1 to about 12, or from 1 to about 6, and the total number of ring members can vary from three to about 15, or from about 3 to about 8. Suitable ring heteroatoms are N, O and S. Suitable hetaryl moieties include, but are not limited to, pyrazolyl, thiophenyl, pyridyl, imidazolyl, tetrazolyl, pyridyl, pyrimidinyl, purinyl, quinazolinyl, quinoxalinyl, benzimidazolyl, benzothiophenyl, etc.

Unless otherwise defined herein, where a moiety is defined as a compound moiety, such as hetarylalkyl (hetaryl and alkyl), aralkyl (aryl and alkyl), etc., each of the sub-moieties is as defined herein.

Unless otherwise defined herein, an electron withdrawing group is a group, such as the cyano or isocyanato group that draws electronic charge away from the carbon to which it is attached. Other electron withdrawing groups of note include those whose electronegativities exceed that of carbon, for example halogen, nitro, or phenyl substituted in the ortho- or para-position with one or more cyano, isothiocyanato, nitro or halo groups.

Unless otherwise defined herein, the terms halogen and halo have their ordinary meanings. Suitable halo (halogen) substituents are Cl, Br, and I.

The aforementioned optional substituents are, unless otherwise herein defined, suitable substituents depending upon desired properties. Included are halogens (Cl, Br, I), alkyl, alkenyl, and alkynyl moieties, NO₂, NH₃ (substituted and unsubstituted), acid moieties (e.g. —CO₂H, —OSO₃H₂, etc.), heterocycloalkyl moieties, hetaryl moieties, aryl moieties, etc.

In all the preceding formulae, the squiggle (˜) indicates a bond to an oxygen or sulfur of the 5′-phosphate. Phosphate protecting groups include those described in U.S. Pat. Nos. 5,760,209, 5,614,621, 6,051,699, 6,020,475, 6,326,478, 6,169,177, 6,121,437, and 6,465,628.

Oligomer Synthesis

Oligomerization of modified and unmodified nucleosides is performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA:Protein Interactions, Ed. Smith (1998), 1-36. Gallo et al., Tetrahedron (2001), 57, 5707-5713) synthesis as appropriate. In addition specific protocols for the synthesis of oligomeric compounds of the invention are illustrated in the examples below.

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

The present invention is also useful for the preparation of oligomeric compounds incorporating at least one 2′-O-protected nucleoside. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound. All 2′-O-protecting groups amenable to the synthesis of oligomeric compounds are included in the present invention. In general a protected nucleoside is attached to a solid support by for example a succinate linker. Then the oligonucleotide is elongated by repeated cycles of deprotecting the 5′-terminal hydroxyl group, coupling of a further nucleoside unit, capping and oxidation (alternatively sulfurization). In a more frequently used method of synthesis the completed oligonucleotide is cleaved from the solid support with the removal of phosphate protecting groups and exocyclic amino protecting groups by treatment with an ammonia solution. Then a further deprotection step is normally required for removal of the more specialized protecting groups used for the protection of 2′-hydroxyl groups thereby affording the fully deprotected oligonucleotide.

A large number of 2′-O-protecting groups have been used for the synthesis of oligoribonucleotides but over the years more effective groups have been discovered. The key to an effective 2′-O-protecting group is that it is capable of selectively being introduced at the 2′-O-position and that it can be removed easily after synthesis without the formation of unwanted side products. The protecting group also needs to be inert to the normal deprotecting, coupling, and capping steps required for oligoribonucleotide synthesis. Some of the protecting groups used initially for oligoribonucleotide synthesis included tetrahydropyran-1-yl and 4-methoxytetrahydropyran-4-yl. These two groups are not compatible with all 5′-O-protecting groups so modified versions were used with 5′-DMT groups such as 1-(2-fluorophenyl)-4-methoxypiperidin-4-yl (Fpmp). Reese has identified a number of piperidine derivatives (like Fpmp) that are useful in the synthesis of oligoribonucleotides including 1-((chloro-4-methyl)phenyl)-4′-methoxypiperidin-4-yl (Reese et al., Tetrahedron Lett., 1986, (27), 2291). Another approach was to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group initially used for the synthesis of oligoribonucleotides was the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc. Perkin I., 2762). The 2′-O-protecting groups can require special reagents for their removal such as for example the t-butyldimethylsilyl group is normally removed after all other cleaving/deprotecting steps by treatment of the oligomeric compound with tetrabutylammonium fluoride (TBAF).

One group of researchers examined a number of 2′-O-protecting groups (Pitsch, S., Chimia, 2001, (55), 320-324.) The group examined fluoride labile and photolabile protecting groups that are removed using moderate conditions. One photolabile group that was examined was the (2-(nitrobenzyl)oxy)methyl (nbm) protecting group (Schwartz et al., Bioorg. Med. Chem. Lett., 1992, (2), 1019.) Other groups examined included a number structurally related formaldehyde acetal-derived, 2′-O-protecting groups. Also prepared were a number of related protecting groups for preparing 2′-O-alkylated nucleoside phosphoramidites including 2′-O-((triisopropylsilyl)oxy)methyl (2′-O—CH₂—O—Si(iPr)₃, TOM). One 2′-O-protecting group that was prepared to be used orthogonally to the TOM group was 2′-O—((R)-1-(2-nitrophenyl)ethyloxy)methyl) ((R)-mnbm).

Another strategy using a fluoride labile 5′-O-protecting group (non-acid labile) and an acid labile 2′-O-protecting group has been reported (Scaringe, Stephen A., Methods, 2001, (23) 206-217). A number of possible silyl ethers were examined for 5′-O-protection and a number of acetals and orthoesters were examined for 2′-O-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

Although a lot of research has focused on the synthesis of oligoribonucleotides the main RNA synthesis strategies that are presently being used commercially include 5′-O-DMT-2′-O-t-butyldimethylsilyl (TBDMS), 5′-O-DMT-2′-O-(1(2-fluorophenyl)-4-methoxypiperidin-4-yl) (FPMP), 2′-O-((triisopropylsilyl)oxy)methyl (2′-O—CH2-O—Si(iPr)3 (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). A current list of some of the major companies currently offering RNA products include Pierce Nucleic Acid Technologies, Dharmacon Research Inc., Ameri Biotechnologies Inc., and Integrated DNA Technologies, Inc. One company, Princeton Separations, is marketing an RNA synthesis activator advertised to reduce coupling times especially with TOM and TBDMS chemistries. Such an activator would also be amenable to the present invention.

The primary groups being used for commercial RNA synthesis are:

TBDMS=5′-O-DMT-2′-O-t-butyldimethylsilyl;

TOM=2′-O-((triisopropylsilyl)oxy)methyl;

DOD/ACE=(5′-O-bis(trimethylsiloxy)cyclododecyloxysilyl ether-2′-O-bis(2-acetoxyethoxy)methyl

FPMP=5′-O-DMT-2′-O-(1(2-fluorophenyl)-4-methoxypiperidin-4-yl).

All of the aforementioned RNA synthesis strategies are amenable to the present invention. Strategies that would be a hybrid of the above e.g. using a 5′-protecting group from one strategy with a 2′-O-protecting from another strategy is also amenable to the present invention.

The preparation of ribonucleotides and oligomeric compounds having at least one ribonucleoside incorporated and all the possible configurations falling in between these two extremes are encompassed by the present invention. The corresponding oligomeric comounds can be hybridized to further oligomeric compounds including oligoribonucleotides having regions of complementarity to form double-stranded (duplexed) oligomeric compounds. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et-al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The methods of preparing oligomeric compounds of the present invention can also be applied in the areas of drug discovery and target validation. The present invention comprehends the use of the oligomeric compounds and targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity is evaluated according to existing literature (Elbashir et al., Nature (2001), 411, 494-498; Nishikura et al., Cell (2001), 107, 415-416; and Bass et al., Cell (2000), 101, 235-238.)

Targets of the Invention

“Targeting” an antisense oligomeric compound to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state, or a nucleic acid molecule from an infectious agent.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid. The terms region, segment, and site can also be used to describe an oligomeric compound of the invention such as for example a gapped oligomeric compound having 3 separate segments.

Since, as is known in the art, the translation initiation codon is typically 5′ AUG (in transcribed mRNA molecules; 5′ ATG in the corresponding DNA molecule), the translation initiation codon is also referred to as the “AUG codon,” the “start codon” or the “AUG start codon.” A minority of genes have a translation initiation codon having the RNA sequence 5′ GUG, 5′ UUG or 5′ CUG, and 5′ AUA, 5′ ACG and 5′ CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. In the context of the invention, “start codon” and “translation initiation codon” refer to the codon or codons that are used in vivo to initiate translation of an mRNA transcribed from a gene encoding a nucleic acid target, regardless of the sequence(s) of such codons. It is also known in the art that a translation termination codon (or “stop codon”) of a gene may have one of three sequences, i.e., 5′ UAA, 5′ UAG and 5′ UGA (the corresponding DNA sequences are 5′ TAA, 5′ TAG and 5′ TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with the antisense oligomeric compounds of the present invention.

The open reading frame (ORF) or “coding region,” which is known in the art to refer to the region between the translation initiation codon and the translation termination codon, is also a region which may be targeted effectively. Within the context of the present invention, a suitable region is the intragenic region encompassing the translation initiation or termination codon of the open reading frame (ORF) of a gene.

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. It is also suitable to target the 5′ cap region.

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence. Targeting splice sites, i.e., intron-exon junctions or exon-intron junctions, may also be particularly useful in situations where aberrant splicing is implicated in disease, or where an overproduction of a particular splice product is implicated in disease. Aberrant fusion junctions due to rearrangements or deletions are also suitable target sites. mRNA transcripts produced via the process of splicing of two (or more) mRNAs from different gene sources are known as “fusion transcripts.” It is also known that introns can be effectively targeted using antisense oligomeric compounds targeted to, for example, DNA or pre-mRNA.

It is also known in the art that alternative RNA transcripts can be produced from the same genomic region of DNA. These alternative transcripts are generally known as “variants”. More specifically, “pre-mRNA variants” are transcripts produced from the same genomic DNA that differ from other transcripts produced from the same genomic DNA in either their start or stop position and contain both intronic and exonic sequences.

Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Within the context of the invention, the types of variants described herein are also suitable target nucleic acids.

The locations on the target nucleic acid to which the oligomeric compounds hybridize are hereinbelow referred to as “suitable target segments.” As used herein, the term “suitable target segment” is defined as at least an 8-nucleobase portion of a target region to which an active oligomeric compound is targeted. While not wishing to be bound by theory, it is presently believed that these target segments represent accessible portions of the target nucleic acid for hybridization.

Exemplary oligomeric compounds include oligomeric compounds that comprise at least the 8 consecutive nucleobases from the 5′-terminus of a targeted nucleic acid e.g. a cellular gene or mRNA transcribed from the gene (the remaining nucleobases being a consecutive stretch of the same oligonucleotide beginning immediately upstream of the 5′-terminus of the antisense compound which is specifically hybridizable to the target nucleic acid and continuing until the oligonucleotide contains from about 8 to about 80 nucleobases). Similarly, oligomeric compounds are represented by oligonucleotide sequences that comprise at least the 8 consecutive nucleobases from the 3′-terminus of one of the illustrative compounds (the remaining nucleobases being a consecutive stretch of the same oligonucleotide beginning immediately downstream of the 3′-terminus of the compound which is specifically hybridizable to the target nucleic acid and continuing until the oligonucleotide contains from about 8 to about 80 nucleobases). One having skill in the art armed with the suitable antisense compounds illustrated herein will be able, without undue experimentation, to identify additional oligomeric compounds.

Once one or more target regions, segments or sites have been identified, antisense oligomeric compounds are chosen which are sufficiently complementary to the target, i.e., hybridize sufficiently well and with sufficient specificity, to give the desired effect.

In accordance with one embodiment of the present invention, a series of compositions of nucleic acid duplexes comprising the oligomeric compounds of the present invention and their complements can b& designed for a specific target or targets. The ends of the strands may be modified by the addition of one or more natural or modified nucleobases to form an overhang. The sense strand of the duplex is then designed and synthesized as the complement of the antisense strand and may also contain modifications or additions to either terminus. For example, in one embodiment, both strands of the duplex would be complementary over the central nucleobases, each having overhangs at one or both termini.

For example, a duplex comprising an antisense oligomeric compound having the sequence CGAGAGGCGGACGGGACCG (SEQ ID NO:3) and having a two-nucleobase overhang of deoxythymidine(dT) would have the following structure:

| | | |
|---|---|---|
| cgagaggcggacgggaccgdTdT | Antisense Strand | (SEQ ID NO:3) |
| \|\|\|\|\|\|\|\|\|\|\|\|\|\|\|\|\|\|\| | | |
| dTdTgctctccgcctgccctggc | Complement Strand | (SEQ ID NO:4) |

RNA strands of the duplex can be synthesized by methods disclosed herein or purchased from various RNA synthesis companies such as for example Dharmacon Research Inc., (Lafayette, Colo.). Once synthesized, the complementary strands are annealed. The single strands are aliquoted and diluted to a concentration of 50 μM. Once diluted, 30 μL of each strand is combined with 15 μL of a 5× solution of annealing buffer. The final concentration of the buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA compound is 20 μM. This solution can be stored frozen (−20° C.) and freeze-thawed up to 5 times.

Once prepared, the desired synthetic duplexs are evaluated for their ability to modulate target expression. When cells reach 80% confluency, they are treated with synthetic duplexs comprising at least one oligomeric compound of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired dsRNA compound at a final concentration of 200 nM. After 5 hours of treatment, the medium is replaced with fresh medium. Cells are harvested 16 hours after treatment, at which time RNA is isolated and target reduction measured by RT-PCR.

In a further embodiment, the “suitable target segments” identified herein may be employed in a screen for additional oligomeric compounds that modulate the expression of a target. “Modulators” are those oligomeric compounds that decrease or increase the expression of a nucleic acid molecule encoding a target and which comprise at least an 8-nucleobase portion which is complementary to a suitable target segment. The screening method comprises the steps of contacting a suitable target segment of a nucleic acid molecule encoding a target with one or more candidate modulators, and selecting for one or more candidate modulators which decrease or increase the expression of a nucleic acid molecule encoding a target. Once it is shown that the candidate modulator or modulators are capable of modulating (e.g. either decreasing or increasing) the expression of a nucleic acid molecule encoding a target, the modulator may then be employed in further investigative studies of the function of a target, or for use as a research, diagnostic, or therapeutic agent in accordance with the present invention.

The suitable target segments of the present invention may also be combined with their respective complementary antisense oligomeric compounds of the present invention to form stabilized double-stranded (duplexed) oligonucleotides.

Hybridization

In the context of this invention, “hybridization” occurs when two sequences come together with enough base complementarity to form a double stranded region. The source of the two sequences can be synthetic or native and can occur in a single strand when the strand has regions of self complementarity. In the present invention, one mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds or between an oligomeric compound and a target nucleic acid. For example, adenine and thymine are complementary nucleobases which pair through the formation of hydrogen bonds. Hybridization can occur under varying circumstances.

An antisense oligomeric compound is specifically hybridizable when binding of the compound to the target nucleic acid interferes with the normal function of the target nucleic acid to cause a loss of activity, and there is a sufficient degree of complementarity to avoid non-specific binding of the antisense oligomeric compound to non-target nucleic acid sequences under conditions in which specific binding is desired, i.e., under physiological conditions in the case of in vivo assays or therapeutic treatment, and under conditions in which assays are performed in the case of in vitro assays.

In the present invention the phrase “stringent hybridization conditions” or “stringent conditions” refers to conditions under which an oligomeric compound of the invention will hybridize to its target sequence, but to a minimal number of other sequences. Stringent conditions are sequence-dependent and will vary with different circumstances and in the context of this invention, “stringent conditions” under which oligomeric compounds hybridize to a target sequence are determined by the nature and composition of the oligomeric compounds and the assays in which they are being investigated.

“Complementary,” as used herein, refers to the capacity for precise pairing of two nucleobases regardless of where the two are located. For example, if a nucleobase at a certain position of an oligomeric compound is capable of hydrogen bonding with a nucleobase at a certain position of a target nucleic acid, the target nucleic acid being a DNA, RNA, or oligonucleotide molecule, then the position of hydrogen bonding between the oligonucleotide and the target nucleic acid is considered to be a complementary position. The oligomeric compound and the further DNA, RNA, or oligonucleotide molecule are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligonucleotide and a target nucleic acid.

It is understood in the art that the sequence of an antisense oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure or hairpin structure). It is suitable that the antisense oligomeric compounds of the present invention comprise at least 70%, at least 80%, at least 90%, at least 95%, or at least 99% sequence complementarity to a target region within the target nucleic acid sequence to which they are targeted. For example, an antisense oligomeric compound in which 18 of 20 nucleobases of the antisense oligomeric compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Screening and Target Validation

In a further embodiment, “suitable target segments” may be employed in a screen for additional oligomeric compounds that modulate the expression of a selected protein. “Modulators” are those oligomeric compounds that decrease or increase the expression of a nucleic acid molecule encoding a protein and which comprise at least an 8-nucleobase portion which is complementary to a suitable target segment. The screening method comprises the steps of contacting a suitable target segment of a nucleic acid molecule encoding a protein with one or more candidate modulators, and selecting for one or more candidate modulators which decrease or increase the expression of a nucleic acid molecule encoding a protein. Once it is shown that the candidate modulator or modulators are capable of modulating (e.g. either decreasing or increasing) the expression of a nucleic acid molecule encoding a peptide, the modulator may then be employed in further investigative studies of the function of the peptide, or for use as a research, diagnostic, or therapeutic agent in accordance with the present invention.

The suitable target segments of the present invention may also be combined with their respective complementary antisense oligomeric compounds of the present invention to form stabilized double-stranded (duplexed) oligonucleotides. Such double stranded oligonucleotide moieties have been shown in the art to modulate target expression and regulate translation as well as RNA processsing via an antisense mechanism. Moreover, the double-stranded moieties may be subject to chemical modifications (Fire et al., Nature, 1998, 391, 806-811; Timmons and Fire, Nature 1998, 395, 854; Timmons et al., Gene, 2001, 263, 103-112; Tabara et al., Science, 1998, 282, 430-431; Montgomery et al., Proc. Natl. Acad. Sci. USA, 1998, 95, 15502-15507; Tuschl et al., Genes Dev., 1999, 13, 3191-3197; Elbashir et al., Nature, 2001, 411, 494-498; Elbashir et al., Genes Dev. 2001, 15, 188-200). For example, such double-stranded moieties have been shown to inhibit the target by the classical hybridization of antisense strand of the duplex to the target, thereby triggering enzymatic degradation of the target (Tijsterman et al., Science, 2002, 295, 694-697).

The compositions comprising oligomeric compounds of the present invention can also be applied in the areas of drug discovery and target validation. The present invention comprehends the use of the oligomeric compounds and suitable targets identified herein in drug discovery efforts to elucidate relationships that exist between proteins and a disease state, phenotype, or condition. These methods include detecting or modulating a target peptide comprising contacting a sample, tissue, cell, or organism with the oligomeric compounds of the present invention, measuring the nucleic acid or protein level of the target and/or a related phenotypic or chemical endpoint at some time after treatment, and optionally comparing the measured value to a non-treated sample or sample treated with a further oligomeric compound of the invention. These methods can also be performed in parallel or in combination with other experiments to determine the function of unknown genes for the process of target validation or to determine the validity of a particular gene product as a target for treatment or prevention of a particular disease, condition, or phenotype.

Effect of nucleoside modifications on RNAi activity is evaluated according to existing literature (Elbashir et al., Nature (2001), 411, 494-498; Nishikura et al., Cell (2001), 107, 415-416; and Bass et al., Cell (2000), 101, 235-238.)

Kits, Research Reagents, Diagnostics, and Therapeutics

The compositions of oligomeric compounds of the present invention can be utilized for diagnostics, therapeutics, prophylaxis and as research reagents and kits. Furthermore, antisense oligonucleotides, which are able to inhibit gene expression with exquisite specificity, are often used by those of ordinary skill to elucidate the function of particular genes or to distinguish between functions of various members of a biological pathway.

For use in kits and diagnostics, the compositions of the present invention, either alone or in combination with other oligomeric compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more antisense oligomeric compounds are compared to control cells or tissues not treated with antisense oligomeric compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds and or oligomeric compounds that affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The compositions of the invention are useful for research and diagnostics in one sense because the oligomeric compounds of the compositions hybridize to nucleic acids encoding proteins. For example, oligonucleotides that are shown to hybridize with such efficiency and under such conditions as disclosed herein as to be effective protein inhibitors will also be effective primers or probes under conditions favoring gene amplification or detection, respectively. These primers and probes are useful in methods requiring the specific detection of nucleic acid molecules encoding proteins and in the amplification of the nucleic acid molecules for detection or for use in further studies. Hybridization of the antisense oligonucleotides, particularly the primers and probes, of the invention with a nucleic acid can be detected by means known in the art. Such means may include conjugation of an enzyme to the oligonucleotide, radiolabelling of the oligonucleotide or any other suitable detection means. Kits using such detection means for detecting the level of selected proteins in a sample may also be prepared.

The specificity and sensitivity of antisense methodologies is also harnessed by those of skill in the art for therapeutic uses. Antisense oligomeric compounds have been employed as therapeutic moieties in the treatment of disease states in animals, including humans. Antisense oligonucleotide drugs, including ribozymes, have been safely and effectively administered to humans and numerous clinical trials are presently underway. It is thus established that antisense oligomeric compounds can be useful therapeutic modalities that can be configured to be useful in treatment regimes for the treatment of cells, tissues and animals, especially humans.

For therapeutics, an animal, such as a human, suspected of having a disease or disorder which can be treated by modulating the expression of a selected protein is treated by administering compositions of the invention in accordance with this invention. For example, in one non-limiting embodiment, the methods comprise the step of administering to the animal in need of treatment, a therapeutically effective amount of a protein inhibitor. The protein inhibitors of the present invention effectively inhibit the activity of the protein or inhibit the expression of the protein. In some embodiments, the activity or expression of a protein in an animal or in vitro is inhibited by at least 10%, by at least 20%, by at least 30%, by at least 40%, by at least 50%, by at least 60%, by at least 70%, by at least 80%, by at least 90%, by at least 95%, by at least 99%, or by 100%. For example, the reduction of the expression of a protein may be measured in serum, adipose tissue, liver or any other body fluid, tissue or organ of the animal. The cells contained within the fluids, tissues or organs being analyzed can contain a nucleic acid molecule encoding a protein and/or the protein itself.

The compositions of the invention can be utilized in pharmaceutical compositions by adding an effective amount to a suitable pharmaceutically acceptable diluent or carrier. Use of the compositions and methods of the invention may also be useful prophylactically.

Formulations

The compositions of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative U.S. patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The compositions of the invention encompass any pharmaceutically acceptable salts, esters, or salts of such esters, or any other compound which, upon administration to an animal, including a human, is capable of providing (directly or indirectly) the biologically active metabolite or residue thereof. Accordingly, for example, the disclosure is also drawn to prodrugs and pharmaceutically acceptable salts of the compositions of the invention, pharmaceutically acceptable salts of such prodrugs, and other bioequivalents. The term “prodrug” indicates a therapeutic agent that is prepared in an inactive form that is converted to an active form (i.e., drug) within the body or cells thereof by the action of endogenous enzymes or other chemicals and/or conditions. In particular, prodrug versions of the oligonucleotides of the invention are prepared as SATE ((S acetyl-2-thioethyl) phosphate) derivatives according to the methods disclosed in WO 93/24510 to Gosselin et al., published Dec. 9, 1993 or in WO 94/26764 and U.S. Pat. No. 5,770,713 to Imbach et al.

The term “pharmaceutically acceptable salts” refers to physiologically and pharmaceutically acceptable salts of the oligomeric compounds of the invention: i.e., salts that retain the desired biological activity of the parent compound and do not impart undesired toxicological effects thereto. For oligonucleotides, suitable examples of pharmaceutically acceptable salts and their uses are further described in U.S. Pat. No. 6,287,860.

The present invention also includes pharmaceutical compositions and formulations which include the compositions of the invention. The pharmaceutical compositions of the present invention may be administered in a number of ways depending upon whether local or systemic treatment is desired and upon the area to be treated. Administration may be topical (including ophthalmic and to mucous membranes including vaginal and rectal delivery), pulmonary, e.g., by inhalation or insufflation of powders or aerosols, including by nebulizer; intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Oligonucleotides with at least one 2′-O-methoxyethyl modification are believed to be particularly useful for oral administration. Pharmaceutical compositions and formulations for topical administration may include transdermal patches, ointments, lotions, creams, gels, drops, suppositories, sprays, liquids and powders. Conventional, pharmaceutical carriers, aqueous, powder or oily bases, thickeners and the like may be necessary or desirable. Coated condoms, gloves and the like may also be useful.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

Emulsions are typically heterogenous systems of one liquid dispersed in another in the form of droplets usually exceeding 0.1 μm in diameter. Emulsions may contain additional components in addition to the dispersed phases, and the active drug which may be present as a solution in either the aqueous phase, oily phase or itself as a separate phase. Microemulsions are included as an embodiment of the present invention. Emulsions and their uses are well known in the art and are further described in U.S. Pat. No. 6,287,860.

Formulations of the present invention include liposomal formulations. As used in the present invention, the term “liposome” means a vesicle composed of amphiphilic lipids arranged in a spherical bilayer or bilayers. Liposomes are unilamellar or multilamellar vesicles which have a membrane formed from a lipophilic material and an aqueous interior that contains the composition to be delivered. Cationic liposomes are positively charged liposomes which are believed to interact with negatively charged DNA molecules to form a stable complex. Liposomes that are pH sensitive or negatively charged are believed to entrap DNA rather than complex with it. Both cationic and noncationic liposomes have been used to deliver DNA to cells.

Liposomes also include “sterically stabilized” liposomes, a term which, as used herein, refers to liposomes comprising one or more specialized lipids that, when incorporated into liposomes, result in enhanced circulation lifetimes relative to liposomes lacking such specialized lipids. Examples of sterically stabilized liposomes are those in which part of the vesicle-forming lipid portion of the liposome comprises one or more glycolipids or is derivatized with one or more hydrophilic polymers, such as a polyethylene glycol (PEG) moiety. Liposomes and their uses are further described in U.S. Pat. No. 6,287,860.

The pharmaceutical formulations and compositions of the present invention may also include surfactants. The use of surfactants in drug products, formulations and in emulsions is well known in the art. Surfactants and their uses are further described in U.S. Pat. No. 6,287,860.

In one embodiment, the present invention employs various penetration enhancers to effect the efficient delivery of nucleic acids, particularly oligonucleotides. In addition to aiding the diffusion of non-lipophilic drugs across cell membranes, penetration enhancers also enhance the permeability of lipophilic drugs. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860.

One of skill in the art will recognize that formulations are routinely designed according to their intended use, i.e. route of administration.

Suitable formulations for topical administration include those in which the oligonucleotides of the invention are in admixture with a topical delivery agent such as lipids, liposomes, fatty acids, fatty acid esters, steroids, chelating agents and surfactants. Suitable lipids and liposomes include neutral (e.g. dioleoylphosphatidyl DOPE ethanolamine, dimyristoylphosphatidyl choline DMPC, distearolyphosphatidyl choline) negative (e.g. dimyristoylphosphatidyl glycerol DMPG) and cationic (e.g. dioleoyltetramethylaminopropyl DOTAP and dioleoylphosphatidyl ethanolamine DOTMA).

For topical or other administration, oligonucleotides of the invention may be encapsulated within liposomes or may form complexes thereto, in particular to cationic liposomes. Alternatively, oligonucleotides may be complexed to lipids, in particular to cationic lipids. Suitable fatty acids and esters, pharmaceutically acceptable salts thereof, and their uses are further described in U.S. Pat. No. 6,287,860. Topical formulations are described in detail in U.S. patent application Ser. No. 09/315,298 filed on May 20, 1999.

Compositions and formulations for oral administration include powders or granules, microparticulates, nanoparticulates, suspensions or solutions in water or non-aqueous media, capsules, gel capsules, sachets, tablets or minitablets. Thickeners, flavoring agents, diluents, emulsifiers, dispersing aids or binders may be desirable. Suitable oral formulations are those in which oligonucleotides of the invention are administered in conjunction with one or more penetration enhancers surfactants and chelators. Suitable surfactants include fatty acids and/or esters or salts thereof, bile acids and/or salts thereof. Suitable bile acids/salts and fatty acids and their uses are further described in U.S. Pat. No. 6,287,860. Also suitable are combinations of penetration enhancers, for example, fatty acids/salts in combination with bile acids/salts. A particularly suitable combination is the sodium salt of lauric acid, capric acid and UDCA. Further penetration enhancers include polyoxyethylene-9-lauryl ether, polyoxyethylene-20-cetyl ether. Oligonucleotides of the invention may be delivered orally, in granular form including sprayed dried particles, or complexed to form micro or nanoparticles. Oligonucleotide complexing agents and their uses are further described in U.S. Pat. No. 6,287,860. Oral formulations for oligonucleotides and their preparation are described in detail in U.S. application Ser. No. 09/108,673 (filed Jul. 1, 1998), Ser. No. 09/315,298 (filed May 20, 1999) and Ser. No. 10/071,822, filed Feb. 8, 2002.

Compositions and formulations for parenteral, intrathecal or intraventricular administration may include sterile aqueous solutions which may also contain buffers, diluents and other suitable additives such as, but not limited to, penetration enhancers, carrier compounds and other pharmaceutically acceptable carriers or excipients.

Certain embodiments of the invention provide pharmaceutical compositions containing one or more of the compositions of the invention and one or more other chemotherapeutic agents which function by a non-antisense mechanism. Examples of such chemotherapeutic agents include but are not limited to cancer chemotherapeutic drugs such as daunorubicin, daunomycin, dactinomycin, doxorubicin, epirubicin, idarubicin, esorubicin, bleomycin, mafosfamide, ifosfamide, cytosine arabinoside, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, cytarabine, 5-azacytidine, hydroxyurea, deoxycoformycin, 4-hydroxyperoxycyclophosphoramide, 5-fluorouracil (5-FU), 5-fluorodeoxyuridine (5-FUdR), methotrexate (MTX), colchicine, taxol, vincristine, vinblastine, etoposide (VP-16), trimetrexate, irinotecan, topotecan, gemcitabine, teniposide, cisplatin and diethylstilbestrol (DES). When used with the compositions of the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribivirin, vidarabine, acyclovir and ganciclovir, may also be combined in compositions of the invention. Combinations of compositions of the invention and other non-antisense drugs are also within the scope of this invention. One or more compositions of the invention can be used in combination with other therapeutic agents to create a coctail as is currently the strategy for certain viral infections.

In another related embodiment, therapeutically effective combination therapies may comprise the use of two or more compositions of the invention wherein the multiple compositions are targeted to a single or multiple nucleic acid targets. Numerous examples of antisense oligomeric compounds are known in the art. Two or more combined compounds may be used together or sequentially

Dosing

The formulation of therapeutic compositions and their subsequent administration (dosing) is believed to be within the skill of those in the art. Dosing is dependent on severity and responsiveness of the disease state to be treated, with the course of treatment lasting from several days to several months, or until a cure is effected or a diminution of the disease state is achieved. Optimal dosing schedules can be calculated from measurements of drug accumulation in the body of the patient. Persons of ordinary skill can easily determine optimum dosages, dosing methodologies and repetition rates. Optimum dosages may vary depending on the relative potency of individual oligonucleotides, and can generally be estimated based on EC50s found to be effective in in vitro and in vivo animal models. In general, dosage is from 0.01 μg to 100 g per kg of body weight, from 0.1 μg to 10 g per kg of body weight, from 1 μg to 1 g per kg of body weight, from 10 μg to 100 mg per kg of body weight, from 100 μg to 10 mg per kg of body weight, or from 100 μg to 1 mg per kg of body weight, and may be given once or more daily, weekly, monthly or yearly, or even once every 2 to 20 years. Persons of ordinary skill in the art can easily estimate repetition rates for dosing based on measured residence times and concentrations of the drug in bodily fluids or tissues. Following successful treatment, it may be desirable to have the patient undergo maintenance therapy to prevent the recurrence of the disease state, wherein the oligonucleotide is administered in maintenance doses, ranging from 0.01 ug to 100 g per kg of body weight, once or more daily, to once every 20 years.

While the present invention has been described with specificity in accordance with certain of its embodiments, the following examples serve only to illustrate the invention and are not intended to limit the same.

In order that the invention disclosed herein may be more efficiently understood, examples are provided below. It should be understood that these examples are for illustrative purposes only and are not to be construed as limiting the invention in any manner. Throughout these examples, molecular cloning reactions, and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Activity of Full 2′-F siRNA Constructs

The activity of unmodified siRNA and the identical siRNA having a full 2′-F strand in the sense, antisense and both strands was determined. The siRNA constructs targeted PTEN mRNA and the activities were compared to the untreated control.

| | | |
|---|---|---|
| SEQ ID NO. | ISIS No | Sequence 5′ to 3′ Targeted to PTEN 116847 site |

| 100 | 271790 | CAAAUCCAGAGGCUAGCAGdTdT (s) |
|---|---|---|
| 100 | 279467 | CfAfAfAfUfCfCfAfGfAfGfGfCfUfAfGfCfAfGfdTdT (s) |
| 101 | 271766 | CUGCUAGCCUCUGGAUUUGdTdT (as) |
| 101 | 279471 | CfUfGfCfUfAfGfCfCfUfCfUfGfGfAfUfUfUfGfdTdT (as) |
| | | (Xf = 2′-F modified nucleoside) |
| SEQ ID NO: | | |
| Antisense:Sense | ISIS No: | PTEN mRNA level (% untreated control) |
| 101:100 (unmodified) | 271766:271790 | 24% (116847 site) |
| 101:100 | 279467:279471 | 78% (116847 site) |
| 101:100 | 279467:271790 | 54% (116847 site) |
| 101:100 | 271766:279471 | 63% (116847 site). |

### Example 2

### Effects of Purine Modifications on siRNA Activity in T24 Cells

In this study siRNA constructs targeting PTEN mRNA in T24 cells were prepared having modified bases selected from inosine (I), 2,6-diaminopurine (DAP), 2 amino purine (2AP) and purine (pu). The activity was compared to the unmodified RNA construct. All the double stranded RNA constructs including the unmodified construct were prepared having dTdT overhangs at the 3′-ends. In the sense column the sense modified strand was hybridized with the unmodified antisense strand. In the antisense column the modified antisense was hybridized to the unmodified sense strand. In the both column, both strands are base modified with the modification listed in the left hand column. The results are listed in the table below.

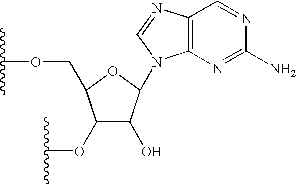

2-Amino Purine (2AP)

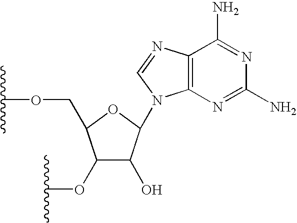

2,6-Diamino Purine (DAP)

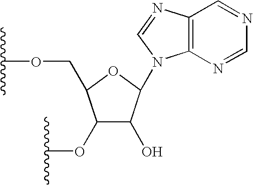

Purine (Pu)

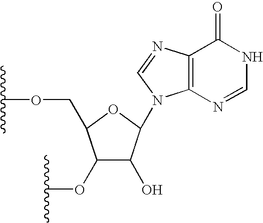

Inosine (I)

| | | Sequence 5′ to 3′ Targeted to |
|---|---|---|
| SEQ ID NO. | ISIS No | PTEN 116847 site |
| 100 (unmodified) | 271790 | CAAAUCCAGAGGCUAGCAGdTdT (s) |
| 101 (unmodified) | 271766 | CUGCUAGCCUCUGGAUUUGdTdT (as) |
| 100 (A = 2AP) | 271383 | CAAAUCCAGAGGCUAGCAGdTdT (s) |
| 101 (A = AP) | 271384 | CUGCUAGCCUCUGGAUUUGdTdT (as) |
| 100 (A = DAP) | 271385 | CAAAUCCAGAGGCUAGCAGdTdT (s) |
| 101 (A = DAP) | 271386 | CUGCUAGCCUCUGGAUUUGdTdT (as) |
| 100 (A = PU) | 271381 | CAAAUCCAGAGGCUAGCAGdTdT (s) |
| 101 (A = PU) | 271382 | CUGCUAGCCUCUGGAUUUGdTdT (as) |
| 100 (I = inosine) | 271387 | CAAAUCCAIAIICUAICAIdTdT (s) |
| 101 (I = inosine) | 271388 | CUICUAICCUCUIIAUUUIdTdT (as) |

Each modified strand was paired with the complement modified strand as shown or with the unmodified strand to give three constructs for each type of modified base nucleoside. The activities are shown below.

Modification PTEN mRNA Level (% Untreated Control)

| | Sense | Antisense | Both |
|---|---|---|---|
| 2AP | 20% | 19% | 18% |
| DAP | 27% | 15% | 21% |
| PU | 53% | 24% | 80% |
| I | 17% | 59% | 67% |
| untreated | 100% | | |
| control | 75% | | |

| | | |
|---|---|---|
| unmodified | 15% | (RNA:RNA). |

### Example 3

### Effects of Base Modified RNA Strands on Activity of Full 2′-F Modified siRNA Constructs

Another study looked at the effect of base modifications on the activity of full 2′-F modified siRNA constructs targeted to PTEN mRNA in T24 cells.

| SEQ ID NO. | ISIS No | Sequence 5′ to 3′ Targeted to PTEN 116847 site |
|---|---|---|
| 100 | 271790 | CAAAUCCAGAGGCUAGCAGdTdT (s) |
| 101 | 271766 | CUGCUAGCCUCUGGAUUUGdTdT (as) |
| 100 (DNA) | 290222 | CAAATCCAGAGGCTAGCAGTT (s) |
| 101 (DNA) | 290221 | CTGCTAGCCTCTGGATTTGTT (as) |
| 100 | 279467 | CfAfAfAfUfCfCfAfGfAfGfGfCfUfAfGfCfAfGdTdT (s) |
| 101 | 279471 | CfUfGfCfUfAfGfCfCfUfCfUfGfGfAfUfUfUfGfdTdT (as) |
| 100 (I = inosine) | 271387 | CAAAUCCAIAIICUAICAIdTdT (s) |
| 101 (I = inosine) | 271388 | CUICUAICCUCUIIAUUUIdTdT (as) |
| 100 (A = Pu) | 271381 | CAAAUCCAGAGGCUAGCAGdTd (s) |
| | | (Xf = 2′-F modified nueleoside) |

| ds construct | PTEN mRNA level (% untreated control) |
|---|---|
| Sense full 2′-F (279467) | |
| antisense - (271766) unmodified RNA | 37% |
| antisense - (271388) I | 100% |
| antisense - (290221) DNA | 92% |
| Antisense full 2′-F (279471) | |
| sense - (271790) unmodified RNA | 54% |
| sense - (271387) I | 37% |
| sense - (290222) DNA | 108% |
| sense - (271381) PU | 82%. |

Sense full 2′-F (279467)

| | |
|---|---|
| antisense - (271766) unmodified RNA | 37% |
| antisense - (271388) I | 100% |
| antisense - (290221) DNA | 92% |

Antisense full 2′-F (279471)

| | |
|---|---|
| sense - (271790) unmodified RNA | 54% |
| sense - (271387) I | 37% |
| sense - (290222) DNA | 108% |
| sense - (271381) PU | 82%. |

### Example 4

### Effect of Duplex Stability on siRNA Activity

In a further study siRNA duplexes were modified to increase the binding affinity/duplex stability over a selected range. The activity of the siRNA's was determined as a function of duplex stability. The siRNA duplexes were prepared having 3′-dTdT overhangs.

| | | |
|---|---|---|
| SEQ ID NO. | ISIS No | Sequence 5′ to 3′ Targeted to PTEN 116847 site |

| | | |
|---|---|---|
| 100 (unmodified) | 271790 | CAAAUCCAGAGGCUAGCAGdTdT (s) |
| 100 | 279467 | CfAfAfAfUfCfCfAfGfAfGfGfCfUfAfGfCfAfGfdTdT (s) |
| 100 (I = inosine) | 271387 | CAAAUCCAIAIICUAICAIdTdT (s) |
| 101 (unmodified) | 271766 | CUGCUAGCCUCUGGAUUUGdTdT (as) |
| 101 | 279471 | CfUfGfCfUfAfGfCfCfUfCfUfGfGfAfUfUfUfGfdTdT (as) |
| | | (Xf = 2′-F modified nucleoside) |

| | | | |
|---|---|---|---|
| SEQ ID NO: (as:s) | siRNA duplex | Tm | mRNA level |

| | | | |
|---|---|---|---|
| 101/100 | 271766:271387 | 57° C. | 26% |
| 101/100 | 271766:271790 | 74° C. | 24% |
| 101/100 | 271766:279467 | 92° C. | 39% |
| 101/100 | 279471:279467 | >100° C. | 67% |
| 101/100 | 279471:271387 | 72° C. | 34% |
| 101/100 | 279471:271790 | 91° C. | 54% |

It was shown that increasing the stability of the siRNA duplex lowers the activity e.g. is less effective in reduction of mRNA levels in the assay.

### Example 5

### Activity of Inosine Analogs of Anti-PTEN siRNA in HUVECS 24 Hours Post-Treatment

A further comparative study examined the effect of the number of inosine base modified nucleosides on the activity of a selected anti-PTEN siRNA construct. The untreated control was designated 100%. The sense strand was kept unmodified (Sense strand, SEQ ID NO:100). The patern of inosine substitution is illustrated below.

| | | |
|---|---|---|
| SEQ ID NO. | ISIS No | Sequence 5′ to 3′ Targeted to PTEN 116847 site |

| 100 (unmodified) | 271790 | CAAAUCCAGAGGCUAGCAGdTdT (s) |
|---|---|---|
| 101 (unmodified) | 271766 | CUGCUAGCCUCUGGAUUUGdTdT (as) |
| 101 (I = inosine) | 271388 | CUICUAICCUCUIIAUUUIdTdT (as) |
| 101 (I = inosine) | 293994 | CUGCUAGCCUCUIIAUUUGdTdT (as) |
| 101 (I = inosine) | 293995 | CUICUAICCUCUGGAUUUGdTdT (as) |
| 101 (I = inosine) | 293996 | CUGCUAGCCUCUGGAUUUIdTdT (as) |
| SEQ ID NO: | | |
| Antisense:Sense | ISIS No: | PTEN mRNA level (% untreated control) |
| 101:100 (unmodified) | 271766:271790 | 9% (116847 site) |
| 101:100 | 271388:271790 | 17% (116847 site) |
| 101:100 | 293994:271790 | 7% (116847 site) |
| 101:100 | 293995:271790 | 11% (116847 site) |
| 101:100 | 293996:271790 | 80% (116847 site). |

### Example 6

### Affinity Modified siRNA's (Base Modified or Sugar Modified) Targeted to PTEN

The activities of 25 siRNA's were determined as compared against unmodified duplex and untreated control. The relative ability to lower mRNA levels in HUVEC cells was determined for each construct.

| | | |
|---|---|---|
| SEQ ID NO. | ISIS No | Sequence 5′ to 3′ Targeted to PTEN 116847 site |

| 101 (unmodified) | 271766 | CUGCUAGCCUCUGGAUUUGdTdT (as) |
|---|---|---|
| 101 (I) | 271388 | CUICUAICCUCUIIAUUUIdTdT (as) |
| 101 (I) | 293997 | CUICUAICCUCUIIAUUUGdTdT (as) |
| 101 (I, A = DAP) | 293998 | CUICUAICCUCUIIAUUUGdTdTd (as) |
| 101 (A = DAP) | 271386 | CUGCUAGCCUCUGGAUUUGdTdT (as) |
| 100 (unmodified) | 271790 | CAAAUCCAGAGGCUAGCAGdTdT (s) |
| 100 | 290224 | CmAmAmAmUmCmCmAmGmAmGmGmCmUmAmGm |
| | | CmAmGmdTdT (s) |
| 100 | 279467 | CfAfAfAfUfCfCfAfGfAfGfGfCfUfAfGfCfAfGdTdT (s) |
| 100 (I) | 271387 | CAAAUCCAIAIICUAICAIdTdT (s) |
| 100 (DAP) | 271385 | CAAAUCCAGAGGCUAGCAGdTdT (s) |
| 100 (A = PU) | 271381 | CAAAUCCAGAGGCUAGCAGdTdT (s) |
| 100 (DNA) | 290222 | CAAATCCAGAGGCTAGCAGTT (s). |
| SEQ ID No: | ISIS No: | |
| Antisense:Sense | Antisense:Sense | PTEN mRNA level (% untreated control) |

| | | |
|---|---|---|
| 101:100 (unmodified) | 271766:271790 | 27% (116847 site) |
| 101:100 | 271766:290224 | 76% (116847 site) |
| 101:100 | 271766:279467 | 35% (116847 site) |
| 101:100 | 271766:271387 | 21% (116847 site) |
| 101:100 | 271766:271385 | 28% (116847 site) |
| 101:100 | 271766:271381 | 53% (116847 site) |
| 101:100 | 271766:290222 | 80% (116847 site) |
| 101:100 | 271388:271790 | 107% (116847 site) |
| 101:100 | 271388:290224 | 129% (116847 site) |
| 101:100 | 271388:279467 | 114% (116847 site) |
| 101:100 | 271388:271387 | 105% (116847 site) |
| 101:100 | 271388:271385 | 60% (116847site) |
| 101:100 | 271388:271381 | 83% (116847 site) |
| 101:100 | 271388:290222 | 113% (116847 site) |
| 101:100 | 293997:271790 | 44% (116847 site) |
| 101:100 | 293997:290224 | 48% (116847 site) |
| 101:100 | 293997:279467 | 33% (116847 site) |
| 101:100 | 293997:271387 | 47% (116847 site) |
| 101:100 | 293997:271385 | 24% (116847 site) |
| 101:100 | 293997:271381 | 100% (116847 site) |
| 101:100 | 293997:290222 | 117% (116847 site) |
| 101:100 | 293998:271790 | 32% (116847 site) |
| 101:100 | 293998:290224 | 54% (116847 site) |
| 101:100 | 293998:279467 | 35% (116847 site) |
| 101:100 | 293998:271387 | 50% (116847 site) |
| 101:100 | 293998:271385 | 26% (116847 site) |
| 101:100 | 293998:271381 | 83% (116847 site) |
| 101:100 | 293998:290222 | 120% (116847 site) |
| 101:100 | 271386:271790 | 27% (116847 site) |
| 101:100 | 271386:290224 | 132% (116847 site) |
| 101:100 | 271386:279467 | 26% (116847 site) |
| 101:100 | 271386:271387 | 29% (116847 site) |
| 101:100 | 271386:271385 | 46% (116847 site). |

### Example 7

### Synthesis of Nucleoside Phosphoramidites

The following compounds, including amidites and their intermediates were prepared as described in U.S. Pat. No. 6,426,220 and published PCT WO 02/36743; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N-4-benzoyl-5-methylcytidine penultimate intermediate for 5-methyl dC amidite, (5′-O-(4,4′-Dimethoxytriphenylmethyl)-2′-deoxy-N-4-benzoyl-5-methylcytidin-3′-O-yl)-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, (5′-O-(4,4′-Dimethoxytriphenylmethyl)-2′-O-(2-methoxyethyl)-5-methyluridin-3′-O-yl)-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N4-benzoyl-5-methyl-cytidine penultimate intermediate, (5′-O-(4,4′-Dimethoxytriphenylmethyl)-2′-O-(2-methoxyethyl)-N4-benzoyl-5-methylcytidin-3′-O-yl)-2-cyanoethyl-N,N-diisopropylphosphoramidite (MOE 5-Me-C amidite), (5′-O-(4,4′-Dimethoxytriphenylmethyl)-2′-O-(2-methoxyethyl)-N6-benzoyladenosin-3′-O-yl)-2-cyanoethyl-N,N-diisopropylphosphoramidite (MOE A amdite), (5′-O-(4,4′-Dimethoxytriphenylmethyl)-2′-O-(2-methoxyethyl)-N4-isobutyrylguanosin-3′-O-yl)-2-cyanoethyl-N,N-diisopropylphosphoramidite (MOE G amidite), 2′-O-(Aminooxyethyl) nucleoside amidites and 2′-O-(dimethylaminooxyethyl) nucleoside amidites, 2′-(Dimethylaminooxyethoxy) nucleoside amidites, 5′-O-tert-Butyldiphenylsilyl-O2-2′-anhydro-5-methyluridine, 5′-O-tert-Butyldiphenylsilyl-2′-O-(2-hydroxyethyl)-5-methyluridine, 2′-O-((2-phthalimidoxy)ethyl)-5′-t-butyldiphenylsilyl-5-methyluridine, 5′-O-tert-butyldiphenylsilyl-2′-O-((2-formadoximinooxy)ethyl)-5-methyluridine, 5′-O-tert-Butyldiphenylsilyl-2′-O-(N,N dimethylaminooxyethyl)-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-((2-cyanoethyl)-N,N-diisopropylphosphoramidite), 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-((2-cyanoethyl)-N,N-diisopropylphosphoramidite), 2′-dimethylaminoethoxyethoxy (2′-DMAEOE) nucleoside amidites, 2′-O-(2(2-N,N-dimethylaminoethoxy)ethyl)-5-methyl uridine, 5′-O-dimethoxytrityl-2′-O-(2(2-N,N-dimethylaminoethoxy)-ethyl))-5-methyl uridine and 5′-O-Dimethoxytrityl-2′-O-(2(2-N,N-dimethylaminoethoxy)-ethyl))-5-methyl uridine-3′-O-(cyanoethyl-N,N-diisopropyl)phosphoramidite.

### Example 8

### Oligonucleotide and Oligonucleoside Synthesis

The oligomeric compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Oligonucleotides: Unsubstituted and substituted phosphodiester (P═O) oligonucleotides are synthesized on an automated DNA synthesizer (Applied Biosystems model 394) using standard phosphoramidite chemistry with oxidation by iodine.

Phosphorothioates (P═S) are synthesized similar to phosphodiester oligonucleotides with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 sec and preceded by the normal capping step. After cleavage from the CPG column and deblocking in concentrated ammonium hydroxide at 55° C. (12-16 hr), the oligonucleotides were recovered by precipitating with >3 volumes of ethanol from a 1 M NH4OAc solution. Phosphinate oligonucleotides are prepared as described in U.S. Pat. No. 5,508,270.

Alkyl phosphonate oligonucleotides are prepared as described in U.S. Pat. No. 4,469,863.

3′-Deoxy-3′-methylene phosphonate oligonucleotides are prepared as described in U.S. Pat. No. 5,610,289 or 5,625,050.

Phosphoramidite oligonucleotides are prepared as described in U.S. Pat. No. 5,256,775 or U.S. Pat. No. 5,366,878.

Alkylphosphonothioate oligonucleotides are prepared as described in published PCT applications PCT/US94/00902 and PCT/US93/06976 (published as WO 94/17093 and WO 94/02499, respectively).

3′-Deoxy-3′-amino phosphoramidate oligonucleotides are prepared as described in U.S. Pat. No. 5,476,925.

Phosphotriester oligonucleotides are prepared as described in U.S. Pat. No. 5,023,243.

Borano phosphate oligonucleotides are prepared as described in U.S. Pat. Nos. 5,130,302 and 5,177,198.

Oligonucleosides: Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone oligomeric compounds having, for instance, alternating MMI and P═O or P═S linkages are prepared as described in U.S. Pat. Nos. 5,378,825, 5,386,023, 5,489,677, 5,602,240 and 5,610,289.

Formacetal and thioformacetal linked oligonucleosides are prepared as described in U.S. Pat. Nos. 5,264,562 and 5,264,564.

Ethylene oxide linked oligonucleosides are prepared as described in U.S. Pat. No. 5,223,618.

### Example 9

### RNA Synthesis

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA oligonucleotides were synthesized.

RNA oligonucleotides are synthesized in a stepwise fashion. Each nucleotide is added sequentially (3′- to 5′-direction) to a solid support-bound oligonucleotide. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound oligonucleotide using water. The support is then treated with 40% methylamine in water for 10 minutes at 55° C. This releases the RNA oligonucleotides into solution, deprotects the exocyclic amines, and modifies the 2′-groups. The oligonucleotides can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and oligonucleotide synthesis. However, after oligonucleotide synthesis the oligonucleotide is treated with methylamine which not only cleaves the oligonucleotide from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with oligonucleotide synthesis and yet, when subsequently modified, permits deprotection to be carried out under relatively mild aqueous conditions compatible with the final RNA oligonucleotide product.

Additionally, methods of RNA synthesis are well known in the art (Scaringe, S. A. Ph.D. Thesis, University of Colorado, 1996; Scaringe et al., J. Am. Chem. Soc., 1998, 120, 11820-11821; Matteucci et al., J. Am. Chem. Soc., 1981, 103, 3185-3191; Beaucage et al., Tetrahedron Lett., 1981, 22, 1859-1862; Dahl et al., Acta Chem. Scand, 1990, 44, 639-641; Reddy et al., Tetrahedrom Lett., 1994, 25, 4311-4314; Wincott et al., Nucleic Acids Res., 1995, 23, 2677-2684; Griffin et al., Tetrahedron, 1967, 23, 2301-2313; Griffin et al., Tetrahedron, 1967, 23, 2315-2331).

RNA oligomeric compounds (RNA oligonucleotides, antisense or sense) of the present invention can be synthesized by the methods herein (also see oligomer synthesis above) or purchased from Dharmacon Research, Inc (Lafayette, Colo.). Once synthesized, complementary RNA antisense oligomeric compounds can then be annealed by methods known in the art to form double stranded (duplexed) antisense oligomeric compounds. For example, duplexes can be formed by combining 30 μl of each of the complementary strands of RNA oligonucleotides (50 uM RNA oligonucleotide solution) and 15 μl of 5× annealing buffer (100 mM potassium acetate, 30 mM HEPES-KOH pH 7.4, 2 mM magnesium acetate) followed by heating for 1 minute at 90° C., then 1 hour at 37° C. The resulting duplexed antisense oligomeric compounds can be used in kits, assays, screens, or other methods to investigate the role of a target nucleic acid.

### Example 10

### Synthesis of Chimeric Oligonucleotides

Chimeric oligonucleotides, oligonucleosides or mixed oligonucleotides/oligonucleosides of the invention can be of several different types. These include a first type wherein the “gap” segment of linked nucleosides is positioned between 5′ and 3′ “wing” segments of linked nucleosides and a second “open end” type wherein the “gap” segment is located at either the 3′ or the 5′ terminus of the oligomeric compound. Oligonucleotides of the first type are also known in the art as “gapmers” or gapped oligonucleotides. Oligonucleotides of the second type are also known in the art as “hemimers” or “wingmers.”

(2′-Q-Me)-(2′-deoxy)-(2′-O-Me) Chimeric Phosphorothioate Oligonucleotides

Chimeric oligonucleotides having 2′-O-alkyl phosphorothioate and 2′-deoxy phosphorothioate oligonucleotide segments are synthesized using an Applied Biosystems automated DNA synthesizer Model 394, as above. Oligonucleotides are synthesized using the automated synthesizer and 2′-deoxy-5′-dimethoxytrityl-3′-O-phosphoramidite for the DNA portion and 5′-dimethoxytrityl-2′-O-methyl-3′-O-phosphoramidite for 5′ and 3′ wings. The standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH4OH) for 12-16 hr at 55° C. The deprotected oligo is then recovered by an appropriate method (precipitation, column chromatography, volume reduced in vacuo and analyzed spetrophotometrically for yield and for purity by capillary electrophoresis and by mass spectrometry.

(2′-O-(2-Methoxyethyl))-(2′-deoxy)-(2′-O-(Methoxyethyl)) Chimeric Phosphorothioate Oligonucleotides

(2′-O-(2-methoxyethyl))-(2′-deoxy)-(-2′-O-(methoxyethyl)) chimeric phosphorothioate oligonucleotides were prepared as per the procedure above for the 2′-O-methyl chimeric oligonucleotide, with the substitution of 2′-O-(methoxyethyl)amidites for the 2′-O-methyl amidites.

(2′-O-(2-Methoxyethyl)Phosphodiester)-(2′-deoxy Phosphorothioate)-(2′-O-(2-Methoxyethyl) Phosphodiester) Chimeric Oligonucleotides

(2′-O-(2-methoxyethyl phosphodiester)-(2′-deoxy phosphorothioate)-(2′-O-(methoxyethyl)phosphodiester) chimeric oligonucleotides are prepared as per the above procedure for the 2′-O-methyl chimeric oligonucleotide with the substitution of 2′-O-(methoxyethyl)amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides are synthesized according to U.S. Pat. No. 5,623,065.

### Example 11

### Design and Screening of Duplexed Antisense Oligomeric Compounds Directed to a Selected Target

In accordance with the present invention, a series of nucleic acid duplexes comprising the antisense oligomeric compounds of the present invention and their complements can be designed to target a target. The ends of the strands may be modified by the addition of one or more natural or modified nucleobases to form an overhang. The sense strand of the dsRNA is then designed and synthesized as the complement of the antisense strand and may also contain modifications or additions to either terminus. For example, in one embodiment, both strands of the dsRNA duplex would be complementary over the central nucleobases, each having overhangs at one or both termini.

For example, a duplex comprising an antisense strand having the sequence CGAGAGGCGGACGGGACCG (SEQ ID NO:3) and having a two-nucleobase overhang of deoxythymidine(dT) would have the following structure:

| | | |
|---|---|---|
| cgagaggcggacgggaccgTT | Antisense Strane | (SEQ ID NO:3) |
| \|\|\|\|\|\|\|\|\|\|\|\|\|\|\|\|\|\|\| | | |
| TTgctctccgcctgccctggc | Complement Strand | (SEQ ID NO:4) |

RNA strands of the duplex can be synthesized by methods disclosed herein or purchased from Dharmacon Research Inc., (Lafayette, Colo.). Once synthesized, the complementary strands are annealed. The single strands are aliquoted and diluted to a concentration of 50 uM. Once diluted, 30 μL of each strand is combined with 15 μL of a 5× solution of annealing buffer. The final concentration of said buffer is 100 mM potassium acetate, 30 mM HEPES-KOH pH 7.4, and 2 mM magnesium acetate. The final volume is 75 μL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM. This solution can be stored frozen (−20° C.) and freeze-thawed up to 5 times.

Once prepared, the duplexed antisense oligomeric compounds are evaluated for their ability to modulate a target expression.

When cells reached 80% confluency, they are treated with duplexed antisense oligomeric compounds of the invention. For cells grown in 96-well plates, wells are washed once with 200 μL OPTI-MEM-1 reduced-serum medium (Gibco BRL) and then treated with 130 μL of OPTI-MEM-1 containing 12 μg/mL LIPOFECTIN (Gibco BRL) and the desired duplex antisense oligomeric compound at a final concentration of 200 nM. After 5 hours of treatment, the medium is replaced with fresh medium. Cells are harvested 16 hours after treatment, at which time RNA is isolated and target reduction measured by RT-PCR.

### Example 12

### Oligonucleotide Isolation

After cleavage from the controlled pore glass solid support and deblocking in concentrated ammonium hydroxide at 55° C. for 12-16 hours, the oligonucleotides or oligonucleosides are recovered by precipitation out of 1 M NH₄OAc with >3 volumes of ethanol. Synthesized oligonucleotides were analyzed by electrospray mass spectroscopy (molecular weight determination) and by capillary gel electrophoresis and judged to be at least 70% full length material. The relative amounts of phosphorothioate and phosphodiester linkages obtained in the synthesis was determined by the ratio of correct molecular weight relative to the −16 amu product (+/−32+/−48). For some studies oligonucleotides were purified by HPLC, as described by Chiang et al., J. Biol. Chem. 1991, 266, 18162-18171. Results obtained with HPLC-purified material were similar to those obtained with non-HPLC purified material.

### Example 13

### Oligonucleotide Synthesis—96 Well Plate Format

Oligonucleotides were synthesized via solid phase P(III) phosphoramidite chemistry on an automated synthesizer capable of assembling 96 sequences simultaneously in a 96-well format. Phosphodiester internucleotide linkages were afforded by oxidation with aqueous iodine. Phosphorothioate internucleotide linkages were generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites were purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Oligonucleotides were cleaved from support and deprotected with concentrated NH4OH at elevated temperature (55-60° C.) for 12-16 hours and the released product then dried in vacuo. The dried product was then re-suspended in sterile water to afford a master plate from which all analytical and test plate samples are then diluted utilizing robotic pipettors.

### Example 14

### Oligonucleotide Analysis Using 96-Well Plate Format

The concentration of oligonucleotide in each well was assessed by dilution of samples and UV absorption spectroscopy. The full-length integrity of the individual products was evaluated by capillary electrophoresis (CE) in either the 96-well format (Beckman P/ACE□ MDQ) or, for individually prepared samples, on a commercial CE apparatus (e.g., Beckman P/ACE™ 5000, ABI 270). Base and backbone composition was confirmed by mass analysis of the oligomeric compounds utilizing electrospray-mass spectroscopy. All assay test plates were diluted from the master plate using single and multi-channel robotic pipettors. Plates were judged to be acceptable if at least 85% of the oligomeric compounds on the plate were at least 85% full length.

### Example 15

### Cell Culture and Oligonucleotide Treatment

The effect of oligomeric compounds on target nucleic acid expression can be tested in any of a variety of cell types provided that the target nucleic acid is present at measurable levels. This can be routinely determined using, for example, PCR or Northern blot analysis. The following cell types are provided for illustrative purposes, but other cell types can be routinely used, provided that the target is expressed in the cell type chosen. This can be readily determined by methods routine in the art, for example Northern blot analysis, ribonuclease protection assays, or RT-PCR.

T-24 Cells:

The human transitional cell bladder carcinoma cell line T-24 was obtained from the American Type Culture Collection (ATCC) (Manassas, Va.). T-24 cells were routinely cultured in complete McCoy's 5A basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence. Cells were seeded into 96-well plates (Falcon-Primaria #353872) at a density of 7000 cells/well for use in RT-PCR analysis.

For Northern blotting or other analysis, cells may be seeded onto 100 mm or other standard tissue culture plates and treated similarly, using appropriate volumes of medium and oligonucleotide.

A549 Cells:

The human lung carcinoma cell line A549 was obtained from the American Type Culture Collection (ATCC) (Manassas, Va.). A549 cells were routinely cultured in DMEM basal media (Invitrogen Corporation, Carlsbad, Calif.) supplemented with 10% fetal calf serum (Invitrogen Corporation, Carlsbad, Calif.), penicillin 100 units per mL, and streptomycin 100 micrograms per mL (Invitrogen Corporation, Carlsbad, Calif.). Cells were routinely passaged by trypsinization and dilution when they reached 90% confluence.

NHDF Cells:

Human neonatal dermal fibroblast (NHDF) were obtained from the Clonetics Corporation (Walkersville, Md.). NHDFs were routinely maintained in Fibroblast Growth Medium (Clonetics Corporation, Walkersville, Md.) supplemented as recommended by the supplier. Cells were maintained for up to 10 passages as recommended by the supplier.

HEK Cells:

Human embryonic keratinocytes (HEK) were obtained from the Clonetics Corporation (Walkersville, Md.). HEKs were routinely maintained in Keratinocyte Growth Medium (Clonetics Corporation, Walkersville, Md.) formulated as recommended by the supplier. Cells were routinely maintained for up to 10 passages as recommended by the supplier.

MCF-7 Cells

The human breast carcinoma cell line MCF-7 is obtained from the American Type Culture Collection (Manassas, Va.). These cells contain a wild-type p53 gene. MCF-7 cells are routinely cultured in DMEM low glucose (Gibco/Life Technologies, Gaithersburg, Md.) supplemented with 10% fetal calf serum (Gibco/Life Technologies, Gaithersburg, Md.). Cells are routinely passaged by trypsinization and dilution when they reach 90% confluence. Cells are seeded into 96-well plates (Falcon-Primaria #3872) at a density of 7000 cells/well for treatment with the oligomeric compounds of the invention.

HepB3 Cells

The human hepatoma cell line HepB3 (Hep3B2.1-7) is obtained from the American Type Culture Collection (ATCC-ATCC Catalog # HB-8064) (Manassas, Va.). This cell line was initially derived from a hepatocellular carcinoma of an 8-yr-old black male. The cells are epithelial in morphology and are tumorigenic in nude mice. HepB3 cells are routinely cultured in Minimum Essential Medium (MEM) with Earle's Balanced Salt Solution, 2 mM L-glutamine, 1.5 g/L sodium bicarbonate, 0.1 mM nonessential amino acids, 1.0 mM sodium pyruvate (ATCC #20-2003, Manassas, Va.) and with 10% heat-inactivated fetal bovine serum (Gibco/Life Technologies, Gaithersburg, Md.). Cells are routinely passaged by trypsinization and dilution when they reach 90% confluence.

Primary Mouse Hepatocytes

Primary mouse hepatocytes are prepared from CD-1 mice purchased from Charles River Labs. Primary mouse hepatocytes are routinely cultured in Hepatocyte Attachment Media (Invitrogen Life Technologies, Carlsbad, Calif.) supplemented with 10% Fetal Bovine Serum (Invitrogen Life Technologies, Carlsbad, Calif.), 250 nM dexamethasone (Sigma-Aldrich Corporation, St. Louis, Mo.), 10 nM bovine insulin (Sigma-Aldrich Corporation, St. Louis, Mo.). Cells are seeded into 96-well plates (Falcon-Primaria #353872, BD Biosciences, Bedford, Mass.) at a density of 4000-6000 cells/well for treatment with the oligomeric compounds of the invention.

Treatment with Oligomeric Compounds:

When cells reached 65-75% confluency, they were treated with oligonucleotide. For cells grown in 96-well plates, wells were washed once with 100 μL OPTI-MEM™-1 reduced-serum medium (Invitrogen Corporation, Carlsbad, Calif.) and then treated with 130 μL of OPTI-MEM™-1 containing 3.75 μg/mL LIPOFECTIN™ (Invitrogen Corporation, Carlsbad, Calif.) and the desired concentration of oligonucleotide. Cells are treated and data are obtained in triplicate. After 4-7 hours of treatment at 37° C., the medium was replaced with fresh medium. Cells were harvested 16-24 hours after oligonucleotide treatment.

The concentration of oligonucleotide used varies from cell line to cell line. To determine the optimal oligonucleotide concentration for a particular cell line, the cells are treated with a positive control oligonucleotide at a range of concentrations. For human cells the positive control oligonucleotide is selected from either ISIS 13920 (TCCGTCATCGCTCCTCAGGG, SEQ ID NO:102) which is targeted to human H-ras, or ISIS 18078, (GTGCGCGCGAGCCCGAAATC, SEQ ID NO:103) which is targeted to human Jun-N-terminal kinase-2 (JNK2). Both controls are 2′-O-methoxyethyl gapmers (2′-O-methoxyethyls shown in bold) with a phosphorothioate backbone. For mouse or rat cells the positive control oligonucleotide is ISIS 15770, ATGCATTCTGCCCCCAAGGA, SEQ ID NO:104, a 2′-O-methoxyethyl gapmer (2′-O-methoxyethyls shown in bold) with a phosphorothioate backbone which is targeted to both mouse and rat c-raf. The concentration of positive control oligonucleotide that results in 80% inhibition of c-H-ras (for ISIS 13920), JNK2 (for ISIS 18078) or c-raf (for ISIS 15770) mRNA is then utilized as the screening concentration for new oligonucleotides in subsequent experiments for that cell line. If 80% inhibition is not achieved, the lowest concentration of positive control oligonucleotide that results in 60% inhibition of c-H-ras, JNK2 or c-raf mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 nM to 300 nM.

### Example 16

### Analysis of Oligonucleotide Inhibition of a Target Expression

Antisense modulation of a target expression can be assayed in a variety of ways known in the art. For example, a target mRNA levels can be quantitated by, e.g., Northern blot analysis, competitive polymerase chain reaction (PCR), or real-time PCR RT-PCR). Real-time quantitative PCR is presently suitable. RNA analysis can be performed on total cellular RNA or poly(A)+ mRNA. One method of RNA analysis of the present invention is the use of total cellular RNA as described in other examples herein. Methods of RNA isolation are well known in the art. Northern blot analysis is also routine in the art. Real-time quantitative (PCR) can be conveniently accomplished using the commercially available ABI PRISM™ 7600, 7700, or 7900 Sequence Detection System, available from PE-Applied Biosystems, Foster City, Calif. and used according to manufacturer's instructions.

Protein levels of a target can be quantitated in a variety of ways well known in the art, such as immunoprecipitation, Western blot analysis (immunoblotting), enzyme-linked immunosorbent assay (ELISA) or fluorescence-activated cell sorting (FACS). Antibodies directed to a target can be identified and obtained from a variety of sources, such as the MSRS catalog of antibodies (Aerie Corporation, Birmingham, Mich.), or can be prepared via conventional monoclonal or polyclonal antibody generation methods well known in the art.

### Example 17

### Design of Phenotypic Assays and In Vivo Studies for the Use of a Target Inhibitors

Phenotypic Assays

Once a target inhibitors have been identified by the methods disclosed herein, the oligomeric compounds are further investigated in one or more phenotypic assays, each having measurable endpoints predictive of efficacy in the treatment of a particular disease state or condition.

Phenotypic assays, kits and reagents for their use are well known to those skilled in the art and are herein used to investigate the role and/or association of a target in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

In one non-limiting example, cells determined to be appropriate for a particular phenotypic assay (i.e., MCF-7 cells selected for breast cancer studies; adipocytes for obesity studies) are treated with a target inhibitors identified from the in vitro studies as well as control compounds at optimal concentrations which are determined by the methods described above. At the end of the treatment period, treated and untreated cells are analyzed by one or more methods specific for the assay to determine phenotypic outcomes and endpoints.

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Analysis of the geneotype of the cell (measurement of the expression of one or more of the genes of the cell) after treatment is also used as an indicator of the efficacy or potency of the a target inhibitors. Hallmark genes, or those genes suspected to be associated with a specific disease state, condition, or phenotype, are measured in both treated and untreated cells.

In Vivo Studies

The individual subjects of the in vivo studies described herein are warm-blooded vertebrate animals, which includes humans.

The clinical trial is subjected to rigorous controls to ensure that individuals are not unnecessarily put at risk and that they are fully informed about their role in the study.

To account for the psychological effects of receiving treatments, volunteers are randomly given placebo or a target inhibitor. Furthermore, to prevent the doctors from being biased in treatments, they are not informed as to whether the medication they are administering is a a target inhibitor or a placebo. Using this randomization approach, each volunteer has the same chance of being given either the new treatment or the placebo.

Volunteers receive either the a target inhibitor or placebo for eight week period with biological parameters associated with the indicated disease state or condition being measured at the beginning (baseline measurements before any treatment), end (after the final treatment), and at regular intervals during the study period. Such measurements include the levels of nucleic acid molecules encoding a target or a target protein levels in body fluids, tissues or organs compared to pre-treatment levels. Other measurements include, but are not limited to, indices of the disease state or condition being treated, body weight, blood pressure, serum titers of pharmacologic indicators of disease or toxicity as well as ADME (absorption, distribution, metabolism and excretion) measurements.

Information recorded for each patient includes age (years), gender, height (cm), family history of disease state or condition (yes/no), motivation rating (some/moderate/great) and number and type of previous treatment regimens for the indicated disease or condition.

Volunteers taking part in this study are healthy adults (age 18 to 65 years) and roughly an equal number of males and females participate in the study. Volunteers with certain characteristics are equally distributed for placebo and a target inhibitor treatment. In general, the volunteers treated with placebo have little or no response to treatment, whereas the volunteers treated with the a target inhibitor show positive trends in their disease state or condition index at the conclusion of the study.

### Example 18

### RNA Isolation

Poly(A)+ mRNA Isolation

Poly(A)+ mRNA was isolated according to Miura et al., (Clin. Chem., 1996, 42, 1758-1764). Other methods for poly(A)+ mRNA isolation are routine in the art. Briefly, for cells grown on 96-well plates, growth medium was removed from the cells and each well was washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) was added to each well, the plate was gently agitated and then incubated at room temperature for five minutes. 55 μL of lysate was transferred to Oligo d(T) coated 96-well plates (AGCT Inc., Irvine Calif.). Plates were incubated for 60 minutes at room temperature, washed 3 times with 200 μL of wash buffer (10 mM Tris-HCl pH 7.6, 1 mM EDTA, 0.3 M NaCl). After the final wash, the plate was blotted on paper towels to remove excess wash buffer and then air-dried for 5 minutes. 60 μL of elution buffer (5 mM Tris-HCl pH 7.6), preheated to 70° C., was added to each well, the plate was incubated on a 90° C. hot plate for 5 minutes, and the eluate was then transferred to a fresh 96-well plate.

Cells grown on 100 mm or other standard plates may be treated similarly, using appropriate volumes of all solutions.

Total RNA Isolation

Total RNA was isolated using an RNEASY 96™ kit and buffers purchased from Qiagen Inc. (Valencia, Calif.) following the manufacturer's recommended procedures. Briefly, for cells grown on 96-well plates, growth medium was removed from the cells and each well was washed with 200 μL cold PBS. 150 μL Buffer RLT was added to each well and the plate vigorously agitated for 20 seconds. 150 μL of 70% ethanol was then added to each well and the contents mixed by pipetting three times up and down. The samples were then transferred to the RNEASY 96™ well plate attached to a QIAVAC™ manifold fitted with a waste collection tray and attached to a vacuum source. Vacuum was applied for 1 minute. 500 μL of Buffer RW1 was added to each well of the RNEASY 96™ plate and incubated for 15 minutes and the vacuum was again applied for 1 minute. An additional 500 μL of Buffer RW1 was added to each well of the RNEASY 96™ plate and the vacuum was applied for 2 minutes. 1 mL of Buffer RPE was then added to each well of the RNEASY 96™ plate and the vacuum applied for a period of 90 seconds. The Buffer RPE wash was then repeated and the vacuum was applied for an additional 3 minutes. The plate was then removed from the QIAVAC™ manifold and blotted dry on paper towels. The plate was then re-attached to the QIAVAC™ manifold fitted with a collection tube rack containing 1.2 mL collection tubes. RNA was then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

### Example 19

### Real-Time Quantitative PCR Analysis of a Target mRNA Levels

Quantitation of a target mRNA levels was accomplished by real-time quantitative PCR using the ABI PRISM™ 7600, 7700, or 7900 Sequence Detection System (PE-Applied Biosystems, Foster City, Calif.) according to manufacturer's instructions. This is a closed-tube, non-gel-based, fluorescence detection system which allows high-throughput quantitation of polymerase chain reaction (PCR) products in real-time. As opposed to standard PCR in which amplification products are quantitated after the PCR is completed, products in real-time quantitative PCR are quantitated as they accumulate. This is accomplished by including in the PCR reaction an oligonucleotide probe that anneals specifically between the forward and reverse PCR primers, and contains two fluorescent dyes. A reporter dye (e.g., FAM or JOE, obtained from either PE-Applied Biosystems, Foster City, Calif., Operon Technologies Inc., Alameda, Calif. or Integrated DNA Technologies Inc., Coralville, Iowa) is attached to the 5′ end of the probe and a quencher dye (e.g., TAMRA, obtained from either PE-Applied Biosystems, Foster City, Calif., Operon Technologies Inc., Alameda, Calif. or Integrated DNA Technologies Inc., Coralville, Iowa) is attached to the 3′ end of the probe. When the probe and dyes are intact, reporter dye emission is quenched by the proximity of the 3′ quencher dye. During amplification, annealing of the probe to the target sequence creates a substrate that can be cleaved by the 5′-exonuclease activity of Taq polymerase. During the extension phase of the PCR amplification cycle, cleavage of the probe by Taq polymerase releases the reporter dye from the remainder of the probe (and hence from the quencher moiety) and a sequence-specific fluorescent signal is generated. With each cycle, additional reporter dye molecules are cleaved from their respective probes, and the fluorescence intensity is monitored at regular intervals by laser optics built into the ABI PRISM™ Sequence Detection System. In each assay, a series of parallel reactions containing serial dilutions of mRNA from untreated control samples generates a standard curve that is used to quantitate the percent inhibition after antisense oligonucleotide treatment of test samples.

Prior to quantitative PCR analysis, primer-probe sets specific to the target gene being measured are evaluated for their ability to be “multiplexed” with a GAPDH amplification reaction. In multiplexing, both the target gene and the internal standard gene GAPDH are amplified concurrently in a single sample. In this analysis, mRNA isolated from untreated cells is serially diluted. Each dilution is amplified in the presence of primer-probe sets specific for GAPDH only, target gene only (“single-plexing”), or both (multiplexing). Following PCR amplification, standard curves of GAPDH and target mRNA signal as a function of dilution are generated from both the single-plexed and multiplexed samples. If both the slope and correlation coefficient of the GAPDH and target signals generated from the multiplexed samples fall within 10% of their corresponding values generated from the single-plexed samples, the primer-probe set specific for that target is deemed multiplexable. Other methods of PCR are also known in the art.

PCR reagents were obtained from Invitrogen Corporation, (Carlsbad, Calif.). RT-PCR reactions were carried out by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by real time RT-PCR are normalized using either the expression level of GAPDH, a gene whose expression is constant, or by quantifying total RNA using RiboGreen™ (Molecular Probes, Inc. Eugene, Oreg.). GAPDH expression is quantified by real time RT-PCR, by being run simultaneously with the target, multiplexing, or separately. Total RNA is quantified using RiboGreen™ RNA quantification reagent (Molecular Probes, Inc. Eugene, Oreg.). Methods of RNA quantification by RiboGreen™ are taught in Jones, L. J., et al, (Analytical Biochemistry, 1998, 265, 368-374).

In this assay, 170 μL of RiboGreen™ working reagent (RiboGreen™ reagent diluted 1:350 in 10 mM Tris-HCl, 1 mM EDTA, pH 7.5) is pipetted into a 96-well plate containing 30 μL purified, cellular RNA. The plate is read in a CytoFluor 4000 (PE Applied Biosystems) with excitation at 485 nm and emission at 530 nm.

Probes and are designed to hybridize to a human a target sequence, using published sequence information.

### Example 20

### Northern Blot Analysis of a Target mRNA Levels

Eighteen hours after antisense treatment, cell monolayers were washed twice with cold PBS and lysed in 1 mL RNAZOL™ (TEL-TEST “B” Inc., Friendswood, Tex.). Total RNA was prepared following manufacturer's recommended protocols. Twenty micrograms of total RNA was fractionated by electrophoresis through 1.2% agarose gels containing 1.1% formaldehyde using a MOPS buffer system (AMRESCO, Inc. Solon, Ohio). RNA was transferred from the gel to HYBOND™-N+ nylon membranes (Amersham Pharmacia Biotech, Piscataway, N.J.) by overnight capillary transfer using a Northern/Southern Transfer buffer system (TEL-TEST “B” Inc., Friendswood, Tex.). RNA transfer was confirmed by UV visualization. Membranes were fixed by UV cross-linking using a STRATALINKER™ UV Crosslinker 2400 (Stratagene, Inc, La Jolla, Calif.) and then probed using QUICKHYB™ hybridization solution (Stratagene, La Jolla, Calif.) using manufacturer's recommendations for stringent conditions.

To detect human a target, a human a target specific primer probe set is prepared by PCR To normalize for variations in loading and transfer efficiency membranes are stripped and probed for human glyceraldehyde-3-phosphate dehydrogenase (GAPDH) RNA (Clontech, Palo Alto, Calif.).

Hybridized membranes were visualized and quantitated using a PHOSPHORIMAGER™ and IMAGEQUANT™ Software V3.3 (Molecular Dynamics, Sunnyvale, Calif.). Data was normalized to GAPDH levels in untreated controls.

### Example 21

### Inhibition of Human a Target Expression by Oligomeric Compounds

In accordance with the present invention, a series of oligomeric compounds are designed to target different regions of the human target RNA. The oligomeric compounds are analyzed for their effect on human target mRNA levels by quantitative real-time PCR as described in other examples herein. Data are averages from three experiments. The target regions to which these sequences are complementary are herein referred to as “suitable target segments” and are therefore suitable for targeting by oligomeric compounds of the present invention. The sequences represent the reverse complement of the suitable oligomeric compounds.

As these “suitable target segments” have been found by experimentation to be open to, and accessible for, hybridization with the oligomeric compounds of the present invention, one of skill in the art will recognize or be able to ascertain, using no more than routine experimentation, further embodiments of the invention that encompass other oligomeric compounds that specifically hybridize to these suitable target segments and consequently inhibit the expression of a target.

According to the present invention, oligomeric compounds include antisense oligomeric compounds, antisense oligonucleotides, ribozymes, external guide sequence (EGS) oligonucleotides, alternate splicers, primers, probes, and other short oligomeric compounds which hybridize to at least a portion of the target nucleic acid.

### Example 22

### Western Blot Analysis of Target Protein Levels

Western blot analysis (immunoblot analysis) is carried out using standard methods. Cells are harvested 16-20 h after oligonucleotide treatment, washed once with PBS, suspended in Laemmli buffer (100 μl/well), boiled for 5 minutes and loaded on a 16% SDS-PAGE gel. Gels are run for 1.5 hours at 150 V, and transferred to membrane for western blotting. Appropriate primary antibody directed to a target is used, with a radiolabeled or fluorescently labeled secondary antibody directed against the primary antibody species. Bands are visualized using a PHOSPHORIMAGER™ (Molecular Dynamics, Sunnyvale Calif.).

### Example 23

### Liposome-Mediated Treatment with Oligomeric Compounds of the Invention

When cells reach the desired confluency, they can be treated with the oligomeric compounds of the invention by liposome-mediated transfection. For cells grown in 96-well plates, wells are washed once with 200 μL OPTI-MEM™-1 reduced-serum medium (Gibco BRL) and then treated with 100 μL of OPTI-MEM™-1 containing 2.5 μg/mL LIPOFECTIN™ (Gibco BRL) and the oligomeric compounds of the invention at the desired final concentration. After 4 hours of treatment, the medium is replaced with fresh medium. Cells are harvested 16 hours after treatment with the oligomeric compounds of the invention for target mRNA expression analysis by real-time PCR.

### Example 24

### Electroporation-Mediated Treatment with Oligomeric Compounds of the Invention

When the cells reach the desired confluency, they can be treated with the oligomeric compounds of the invention by electorporation. Cells are electroporated in the presence of the desired concentration of an oligomeric compound of the invention in 1 mm cuvettes at a density of 1×10⁷ cells/mL, a voltage of 75V and a pulse length of 6 ms. Following the delivery of the electrical pulse, cells are replated for 16 to 24 hours. Cells are then harvested for target mRNA expression analysis by real-time PCR.

### Example 25

### Apoptosis Assay

Caspase-3 activity is evaluated with an fluorometric HTS Caspase-3 assay (Oncogene Research Products, San Diego, Calif.) that detects cleavage after aspartate residues in the peptide sequence (DEVD). The DEVD substrate is labeled with a fluorescent molecule, which exhibits a blue to green shift in fluorescence upon cleavage. Active caspase-3 in treated cells is measured by this assay according to the manufacturer's instructions. Following treatment with the oligomeric compounds of the invention, 50 μL of assay buffer is added to each well, followed by addition 20 μL of the caspase-3 fluorescent substrate conjugate. Data are obtained in triplicate. Fluorescence in wells is immediately detected (excitation/emission 400/505 nm) using a fluorescent plate reader (SpectraMAX GeminiXS, Molecular Devices, Sunnyvale, Calif.). The plate is covered and incubated at 37° C. for an additional three hours, after which the fluorescence is again measured (excitation/emission 400/505 nm). The value at time zero is subtracted from the measurement obtained at 3 hours. The measurement obtained from the untreated control cells is designated as 100% activity.

### Example 26

### Cell Proliferation and Viability Assay

Cell viability and proliferation are measured using the CyQuant Cell Proliferation Assay Kit (Molecular Probes, Eugene, Oreg.) utilizing the CyQuant GR green fluorescent dye which exhibits strong fluorescence enhancement when bound to cellular nucleic acids. The assay is performed according to the manufacturer's instructions. After the treatment with one or more oligomeric compounds of the invention, the microplate is gently inverted to remove the medium from the wells, which are each washed once with 200 μL of phosphate-buffered saline. Plates are frozen at −70° C. and then thawed. A volume of 200 μL of the CyQUANT GR dye/cell-lysis buffer is added to each well. The microplate is incubated for 5 minutes at room temperature, protected from light. Data are obtained in triplicate. Fluorescence in wells is immediately detected (excitation/emission 480/520 nm) using a fluorescent plate reader (SpectraMAX GeminiXS, Molecular Devices, Sunnyvale, Calif.). The measurement obtained from the untreated control cells is designated as 100% activity.

### Example 27

### Leptin-Deficient Mice: a Model of Obesity and Diabetes (ob/ob Mice)

Leptin is a hormone produced by fat that regulates appetite. Deficiencies in this hormone in both humans and non-human animals leads to obesity. ob/ob mice have a mutation in the leptin gene which results in obesity and hyperglycemia. As such, these mice are a useful model for the investigation of obesity and diabetes and treatments designed to treat these conditions. ob/ob mice have higher circulating levels of insulin and are less hyperglycemic than db/db mice, which harbor a mutation in the leptin receptor. In accordance with the present invention, the oligomeric compounds of the invention are tested in the ob/ob model of obesity and diabetes.

Seven-week old male C57Bl/6J-Lepr ob/ob mice (Jackson Laboratory, Bar Harbor, Me.) are fed a diet with a fat content of 10-15% and are subcutaneously injected with the oligomeric compounds of the invention or a control compound at a dose of 25 mg/kg two times per week for 4 weeks. Saline-injected animals, leptin wildtype littermates (i.e. lean littermates) and ob/ob mice fed a standard rodent diet serve as controls. After the treatment period, mice are sacrificed and target levels are evaluated in liver, brown adipose tissue (BAT) and white adipose tissue (WAT). RNA isolation and target mRNA expression level quantitation are performed as described by other examples herein.

To assess the physiological effects resulting from inhibition of target mRNA, the ob/ob mice are further evaluated at the end of the treatment period for serum lipids, serum free fatty acids, serum cholesterol (CHOL), liver triglycerides, fat tissue triglycerides and liver enzyme levels. Hepatic steatosis, or clearing of lipids from the liver, is assessed by measuring the liver triglyceride content. Hepatic steatosis is assessed by routine histological analysis of frozen liver tissue sections stained with oil red O stain, which is commonly used to visualize lipid deposits, and counterstained with hematoxylin and eosin, to visualize nuclei and cytoplasm, respectively.

The effects of target inhibition on glucose and insulin metabolism are evaluated in the ob/ob mice treated with the oligomeric compounds of the invention. Plasma glucose is measured at the start of the treatment and after 2 weeks and 4 weeks of treatment. Plasma insulin is similarly measured at the beginning of the treatment, and following at 2 weeks and at 4 weeks of treatment. Glucose and insulin tolerance tests are also administered in fed and fasted mice. Mice receive intraperitoneal injections of either glucose or insulin, and the blood glucose and insulin levels are measured before the insulin or glucose challenge and at 15, 20 or 30 minute intervals for up to 3 hours.

To assess the metabolic rate of ob/ob mice treated with the oligomeric compounds of the invention, the respiratory quotient and oxygen consumption of the mice are also measured.

The ob/ob mice that received treatment are further evaluated at the end of the treatment period for the effects of target inhibition on the expression genes that participate in lipid metabolism, cholesterol biosynthesis, fatty acid oxidation, fatty acid storage, gluconeogenesis and glucose metabolism. These genes include, but are not limited to, HMG-CoA reductase, acetyl-CoA carboxylase 1 and acetyl-CoA carboxylase 2, carnitine palmitoyltransferase I and glycogen phosphorylase, glucose-6-phosphatase and phosphoenolpyruvate carboxykinase 1, lipoprotein lipase and hormone sensitive lipase. mRNA levels in liver and white and brown adipose tissue are quantitated by real-time PCR as described in other examples herein, employing primer-probe sets that are generated using published sequences of each gene of interest.

### Example 28

### Leptin Receptor-Deficient Mice: a Model of Obesity and Diabetes (db/db Mice)

Leptin is a hormone produced by fat that regulates appetite. Deficiencies in this hormone in both humans and non-human animals leads to obesity. db/db mice have a mutation in the leptin receptor gene which results in obesity and hyperglycemia. As such, these mice are a useful model for the investigation of obesity and diabetes and treatments designed to treat these conditions. db/db mice, which have lower circulating levels of insulin and are more hyperglycemic than ob/ob mice which harbor a mutation in the leptin gene, are often used as a rodent model of type 2 diabetes. In accordance with the present invention, oligomeric compounds of the present invention are tested in the db/db model of obesity and diabetes.

Seven-week old male C57Bl/6J-Lepr db/db mice (Jackson Laboratory, Bar Harbor, Me.) are fed a diet with a fat content of 15-20% and are subcutaneously injected with one or more of the oligomeric compounds of the invention or a control compound at a dose of 25 mg/kg two times per week for 4 weeks. Saline-injected animals, leptin receptor wildtype littermates (i.e. lean littermates) and db/db mice fed a standard rodent diet serve as controls. After the treatment period, mice are sacrificed and target levels are evaluated in liver, brown adipose tissue (BAT) and white adipose tissue (WAT). RNA isolation and target mRNA expression level quantitation are performed as described by other examples herein.

After the treatment period, mice are sacrificed and target levels are evaluated in liver, brown adipose tissue (BAT) and white adipose tissue (WAT). RNA isolation and target mRNA expression level quantitation are performed as described by other examples herein.

To assess the physiological effects resulting from inhibition of target mRNA, the db/db mice that receive treatment are further evaluated at the end of the treatment period for serum lipids, serum free fatty acids, serum cholesterol (CHOL), liver triglycerides, fat tissue triglycerides and liver enzyme levels. Hepatic steatosis, or clearing of lipids from the liver, is assessed by measuring the liver triglyceride content. Hepatic steatosis is also assessed by routine histological analysis of frozen liver tissue sections stained with oil red O stain, which is commonly used to visualize lipid deposits, and counterstained with hematoxylin and eosin, to visualize nuclei and cytoplasm, respectively.

The effects of target inhibition on glucose and insulin metabolism are also evaluated in the db/db mice treated with the oligomeric compounds of the invention. Plasma glucose is measured at the start of the treatment and after 2 weeks and 4 weeks of treatment. Plasma insulin is similarly measured at the beginning of the treatment, and following 2 weeks and 4 weeks of treatment. Glucose and insulin tolerance tests are also administered in fed and fasted mice. Mice receive intraperitoneal injections of either glucose or insulin, and the blood glucose levels are measured before the insulin or glucose challenge and 15, 30, 60, 90 and 120 minutes following the injection.

To assess the metabolic rate of db/db mice treated with the oligomeric compounds of the invention, the respiratory quotient and oxygen consumption of the mice is also measured.

The db/db mice that receive treatment are further evaluated at the end of the treatment period for the effects of target inhibition on the expression genes that participate in lipid metabolism, cholesterol biosynthesis, fatty acid oxidation, fatty acid storage, gluconeogenesis and glucose metabolism. These genes include, but are not limited to, HMG-CoA reductase, acetyl-CoA carboxylase 1 and acetyl-CoA carboxylase 2, carnitine palmitoyltransferase I and glycogen phosphorylase, glucose-6-phosphatase and phosphoenolpyruvate carboxykinase 1, lipoprotein lipase and hormone sensitive lipase. mRNA levels in liver and white and brown adipose tissue are quantitated by real-time PCR as described in other examples herein, employing primer-probe sets that are generated using published sequences of each gene of interest.

### Example 29

### Lean Mice on a Standard Rodent Diet

C57Bl/6 mice are maintained on a standard rodent diet and are used as control (lean) animals. In a further embodiment of the present invention, the oligomeric compounds of the invention are tested in normal, lean animals. Seven-week old male C57Bl/6 mice are fed a diet with a fat content of 4% and are subcutaneously injected with one or more of the oligomeric compounds of the invention or control compounds at a dose of 25 mg/kg two times per week for 4 weeks. Saline-injected animals serve as a control. After the treatment period, mice are sacrificed and target levels are evaluated in liver, brown adipose tissue (BAT) and white adipose tissue (WAT). RNA isolation and target mRNA expression level quantitation are performed as described by other examples herein.

After the treatment period, mice are sacrificed and target levels are evaluated in liver, brown adipose tissue (BAT) and white adipose tissue (WAT). RNA isolation and target mRNA expression level quantitation are performed as described by other examples herein.

To assess the physiological effects resulting from inhibition of target mRNA, the lean mice that receive treatment are further evaluated at the end of the treatment period for serum lipids, serum free fatty acids, serum cholesterol (CHOL), liver triglycerides, fat tissue triglycerides and liver enzyme levels. Hepatic steatosis, or clearing of lipids from the liver, is assessed by measuring the liver triglyceride content. Hepatic steatosis is also assessed by routine histological analysis of frozen liver tissue sections stained with oil red O stain, which is commonly used to visualize lipid deposits, and counterstained with hematoxylin and eosin, to visualize nuclei and cytoplasm, respectively.

The effects of target inhibition on glucose and insulin metabolism are also evaluated in the lean mice treated with the oligomeric compounds of the invention. Plasma glucose is measured at the start of the treatment and after 2 weeks and 4 weeks of treatment. Plasma insulin is similarly measured at the beginning of the treatment, and following 2 weeks and 4 weeks of treatment. Glucose and insulin tolerance tests are also administered in fed and fasted mice. Mice receive intraperitoneal injections of either glucose or insulin, and the blood glucose levels are measured before the insulin or glucose challenge and 15, 30, 60, 90 and 120 minutes following the injection. To assess the metabolic rate of lean mice treated with the oligomeric compounds of the invention, the respiratory quotient and oxygen consumption of the mice is also measured.

The lean mice that received treatment are further evaluated at the end of the treatment period for the effects of target inhibition on the expression genes that participate in lipid metabolism, cholesterol biosynthesis, fatty acid oxidation, fatty acid storage, gluconeogenesis and glucose metabolism. These genes include, but are not limited to, HMG-CoA reductase, acetyl-CoA carboxylase 1 and acetyl-CoA carboxylase 2, carnitine palmitoyltransferase I and glycogen phosphorylase, glucose-6-phosphatase and phosphoenolpyruvate carboxykinase 1, lipoprotein lipase and hormone sensitive lipase. mRNA levels in liver and white and brown adipose tissue are quantitated by real-time PCR as described in other examples herein, employing primer-probe sets that are generated using published sequences of each gene of interest.

Various modifications of the invention, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety. U.S. provisional application Ser. No. 60/480,048 filed Jun. 20, 2003 is incorporated herein by reference in its entirety.

1. A composition comprising a first oligomeric compound and a second oligomeric compound wherein:
at least a portion of the first oligomeric compound is capable of hybridizing with at least a portion of the second oligomeric compound;
at least a portion of the first oligomeric compound is complementary to and capable of hybridizing to a target nucleic acid; and
at least one of the first and the second oligomeric compounds comprises at least one modified nucleoside having enhanced or decreased affinity for the complementary nucleoside in the composition or between the first oligomeric compound and a nucleic acid target; or
one of the first and the second oligomeric compounds comprises at least one modified nucleoside having enhanced affinity for the complementary nucleoside in the composition or between the first oligomeric compound and a nucleic acid target and one of the first and the second oligomeric compounds comprises at least one modified nucleoside having decreased affinity for the complementary nucleoside in the composition or between the first oligomeric compound and a nucleic acid target.

2. The composition of claim 1 wherein the first oligomeric compound comprises at least one modified nucleoside having enhanced affinity for the complementary nucleoside in the composition or between the first oligomeric compound and a nucleic acid target and either the first oligomeric compound or second oligomeric compound comprises at least one modified nucleoside having a decreased affinity for the complementary nucleoside in the composition or between the first oligomeric compound and a nucleic acid target.

3. The composition of claim 1 wherein the first oligomeric compound comprises at least one modified nucleoside having a decreased affinity for the complementary nucleoside in the composition or between the first oligomeric compound and a nucleic acid target, and the second oligomeric compound comprises at least one modified nucleotide having an enhanced affinity for the complementary nucleotide in the first oligomeric compound compared to the affinity of an unmodified nucleotide.

4. The composition of claim 1 wherein the second oligomeric compound comprises at least one modified nucleotide having an enhanced affinity for the complementary nucleotide in the first oligomeric compound compared to the affinity of an unmodified nucleotide, and wherein the second oligomeric compound also comprises at least one modified nucleotide having a decreased affinity for the complementary nucleotide in the first oligomeric compound compared to the affinity of an unmodified nucleotide.

5. The composition of claim 1 wherein the at least one modified nucleotide that comprises an enhanced affinity is a nucleotide comprising a nucleotide base modification.

6. The composition of claim 5 wherein the nucleotide base modification comprises a pyrimidine nucleotide comprising a modification at the 2, 4, 5 or 6 position of the pyrimidine nucleotide.

7. The composition of claim 6 wherein the pyrimidine nucleotide comprises a modification at the 2 or 5 position of the pyrimidine nucleotide.

8. The composition of claim 6 wherein the nucleotide base modification comprises a 2-thio U nucleotide substitution for U nucleotide or 2-thio C nucleotide substitution for a C nucleotide.

9. The composition of claim 6 wherein the nucleotide base modification comprises a 5-alkyl, 5-alkenyl, or 5-alkynyl U substitution for a U nucleotide or 5-alkyl, 5-alkenyl, or 5-alkynyl C substitution for a C nucleotide.

10. The composition of claim 6 wherein the nucleotide base modification comprises a 5-methyl U, 5-methyl C, 5-propynyl U, or 5-propynyl C nucleotide.

11. The composition of claim 5 wherein the nucleotide base modification comprises a pyrimidine nucleotide having a modification, wherein the pyrimidine nucleotide is incorporated as one ring of a multiple ring heterocycle, wherein the multiple ring heterocycle further comprises a phenoxazine moiety, and wherein the multiple ring heterocycle comprises the formula: 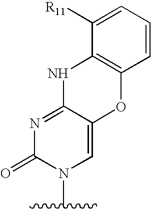
wherein:
R₁₁ is (CH₃)₂N—(CH₂)₂—O—; H₂N—(CH₂)₃—; Ph-CH₂—O—C(═O)—N(H)—(CH₂)₃—; H₂N—; fluorenyl-CH₂—O—C(═O)—N(H)—(CH₂)₃—; phthalimidyl-CH₂—O—C(—O)—N(H)—(CH₂)₃—; Ph-CH₂—O—C(═O)—N(H)—(CH₂)₂—O—; Ph-CH₂—O—C(═O)—N(H)—(CH₂)₃—O—; (CH₃)₂N—N(H)—(CH₂)₂—O—; fluorenyl-CH₂—O—C(—O)—N(H)—(CH₂)₂—O—; fluorenyl-CH₂—O—C(═O)—N(H)—(CH₂)₃—O—; H₂N—(CH₂)₂—O—CH₂—; N₃—(CH₂)₂—O—CH₂—; H₂N—(CH₂)₂—O—; or NH₂C(—NH)NH—.

12-13. (canceled)

14. The composition of claim 5 wherein the nucleotide base modification comprises a purine nucleotide comprising a modification at the 1, 2, 3, 6, 7 or 8 position of the purine nucleotide.

15. The composition of claim 14 wherein the nucleotide base modification comprises a purine nucleotide comprising a modification at the 2, 6 or 7 positions of the purine nucleotide.

16. (canceled)

17. The composition of claim 14 wherein the nucleotide base modification comprises a 2,6-diamino purine substitution for an A nucleotide.

18. The composition of claim 1 wherein the at least one modified nucleotide that comprises an enhanced affinity is a nucleotide comprising a nucleotide sugar modification.

19. The composition of claim 18 wherein the nucleotide sugar modification comprise 2′-F, 2′-MOE, 2′-O-methyl, 2′-O-alkyl, 2′-O-alkenyl, 2′-O-alkynyl, 2′-S-alkyl, 2′-S-alkenyl, 2′-S-alkynyl, 2′-amino, 2′-azido, or 2′-allyl.

20. The composition of claim 1 wherein the at least one modified nucleotide that comprises an enhanced affinity is a nucleotide comprising a modified internucleotide linkage, wherein the modified internucleotide linkage comprises a stabilizing internucleotide linkage, and wherein the stabilizing internucleotide linkage comprises a 3′-deoxy-3′-aminophosphoramidate, 3′-deoxy-3′-methylene phosphonate, 3′-deoxy-3′-aminothiophosphoramidate, acetal, thioacetal, amide-3 and amide-4, MMI, hydrazine, or morpholino.

21-22. (canceled)

23. The composition of claim 1 wherein the at least one modified nucleotide that comprises a decreased affinity is a nucleotide comprising a nucleotide base modification, a nucleotide sugar modification or at least one modified internucleotide linkage.

24. The composition of claim 23 wherein the nucleotide base modification comprises an inosine nucleotide or a purine ribofuranosyl nucleotide.

25. (canceled)

26. The composition of claim 23 wherein the sugar modification comprises a 2′-endo sugar.

27. (canceled)

28. The composition of claim 23 wherein the modified internucleotide linkage comprises a destabilizing internucleotide linkage.

29. The composition of claim 28 wherein the destabilizing internucleotide linkage comprises a phosphorothioate, phosphorodithioate, phosphoramidate, phosphotriester, or alkyl phosphonate internucleotide linkage.

30. The composition of claim 5 wherein the nucleotide base modification comprises a 2′-substituent group which is, independently, F, —O—CH₂CH₂—O—CH₃, —O—C₁-C₁₂ alkyl, —O—CH₂—CH₂—CH₂—NH₂, —O—(CH₂)₂—O—N(R₁)₂, —O—CH₂C(═O)—N(R₁)₂, —O—(CH₂)₂—O—(CH₂)₂—N(R)₂, —O—CH₂—CH₂—CH₂—NHR₁, —O—CF₃, —N₃, —O—CH₂—CH═CH₂, —NHCOR₁, —NH₂, —NHR₁, —N(R₁)₂, —SH, —SR₁, —N(H)OH, —N(H)OR₁, —N(R₁)OH, —N(R₁)OR₁ or —O—CH₂—N(H)—C(═NR₁)(N(R₁)₂);
wherein each R₁ is, independently, H, C₁-C₁₂ alkyl, a protecting group, or substituted or unsubstituted C₁-C₁₂ alkyl, C₂-C₁₂ alkenyl, or C₂-C₁₂ alkynyl, wherein the substituent groups are halogen, hydroxyl, amino, azido, cyano, haloalkyl, alkenyl, alkoxy, thioalkoxy, haloalkoxy, or aryl.

31. The composition of claim 30 wherein each of the 2′-substituent groups is, independently, —F, —O—CH₃, —O—CH₂CH₂—O—CH₃, —O—CH₂—CH═CH₂, —O—CF₃, N₃, NH₂, NHOH, —O—(CH₂)₂—O—N(R₁)₂, —O—CH₂C(O)—N(R₁)₂, —O—CH₂—CH₂—CH₂—NH₂, —O—(CH₂)₂—O—(CH₂)₂—N(R₁)₂ or —O—CH₂—N(H)—C(═NR₁)(N(R₁)₂);
wherein each R₁ is, independently, H, C₁-C₁₂ alkyl, a protecting group, or substituted or unsubstituted C₁-C₁₂ alkyl, C₂-C₁₂ alkenyl, or C₂-C₁₂ alkynyl, wherein the substituent groups are halogen, hydroxyl, amino, azido, cyano, haloalkyl, alkenyl, alkoxy, thioalkoxy, haloalkoxy, or aryl.

32. The composition of claim 31 wherein each of the 2′-substituent groups is, independently, —F, —O—CH₂CH₂—O—CH₃, —O—CH₃, —O—CH₂—CH═CH₂, —O—CF₃ or —O—CH₂—CH—CH₂—NH(Rⱼ) where Rⱼ is H or C₁-C₁₀ alkyl.

33. The composition of claim 32 wherein each of the 2′-substituent groups is, independently, F, —O—CH₃, —O—CF₃, or —O—CH₂CH₂—O—CH₃.

34. The composition of claim 5 wherein at least one modified nucleotide base is a locked nucleic acid (LNA).

35. The composition of claim 18 wherein the nucleotide sugar modification is, independently, C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—Ra), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol, or a polyether of the formula (—O-alkyl)ₘₐ;
wherein each Rₐ is, independently, hydrogen, a protecting group, or substituted or unsubstituted alkyl, alkenyl, or alkynyl, wherein the substituent groups are haloalkyl, alkenyl, alkoxy, thioalkoxy, haloalkoxy, aryl, halogen, hydroxyl, amino, azido, carboxy, cyano, nitro, mercapto, a sulfide group, a sulfonyl group, or a sulfoxide group;
or each sugar substituent group has one of formula Ia or IIa: 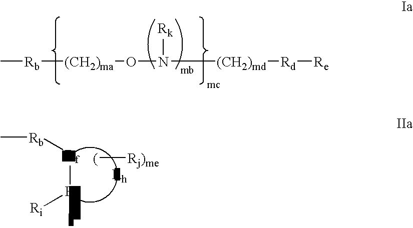
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIa; 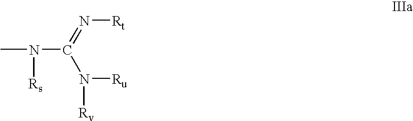
Rₚ and R_{q} are each, independently, hydrogen or C₁-C₁₀ alkyl;
Rᵣ is —Rₓ-R_{y};
each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, or a conjugate group, wherein the substituent groups are hydroxyl, amino, alkoxy, carboxy, benzyl, phenyl, nitro, thiol, thioalkoxy, halogen, alkyl, aryl, alkenyl, or alkynyl;
or, optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl, or aryl;
Rₓ is a bond or a linking moiety;
R_{y} is a chemical functional group, a conjugate group or a solid support medium;
Rₖ is hydrogen, a nitrogen protecting group or —Rₓ-R_{y};
each Rₘ and Rₙ is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are hydroxyl, amino, alkoxy, carboxy, benzyl, phenyl, nitro, thiol, thioalkoxy, halogen, alkyl, aryl, alkenyl, alkynyl, NH3+, N(Rᵤ)(Rᵥ), guanidine, or acyl where the acyl is an acid amide or an ester;
or Rₘ and Rₙ, together, are a nitrogen protecting group, are joined in a ring structure that optionally includes an additional heteroatom selected from N and O, or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein the heteroatoms are oxygen, nitrogen, or sulfur and wherein the ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;
ma is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.

36-37. (canceled)

38. The composition of claim 1 wherein each of the first and second oligomeric compounds comprises from about 12 to about 30 nucleobases.

39. (canceled)

40. The composition of claim 1 wherein each of the first and second oligomeric compounds comprises from about 19 to about 23 nucleobases.

41-50. (canceled)

51. The composition of claim 23 wherein the nucleotide base modification comprises a 2′-substituent group which is, independently, F, —O—CH₂CH₂—O—CH₃, —O—C₁-C₁₂ alkyl, —O—CH₂—CH₂—CH₂—NH₂, —O—(CH₂)₂—O—N(R₁)₂, —O—CH₂C(═O)—N(R₁)₂, —O—(CH₂)₂—O—(CH₂)₂—N(R₁)₂, —O—CH₂—CH₂—CH₂—NHR₁, —O—CF₃, —N₃, —O—CH₂—CH═CH₂, —NHCOR₁, —NH₂, —NHR₁, —N(R₁)₂, —SH, —SR₁, —N(H)OH, —N(H)OR₁, —N(R₁)OH, —N(R₁)OR₁ or —O—CH₂—N(H)—C(═NR₁)(N(R₁)₂);
wherein each R₁ is, independently, H, C₁-C₁₂ alkyl, a protecting group, or substituted or unsubstituted C₁-C₁₂ alkyl, C₂-C₁₂ alkenyl, or C₂-C₁₂ alkynyl, wherein the substituent groups are halogen, hydroxyl, amino, azido, cyano, haloalkyl, alkenyl, alkoxy, thioalkoxy, haloalkoxy, or aryl.

52. The composition of claim 51 wherein each of the 2′-substituent groups is, independently, —F, —O—CH₃, —O—CH₂CH₂—O—CH₃, —O—CH₂—CH═CH₂, —O—CF₃, N₃, NH₂, NHOH, —O—(CH₂)₂—O—N(R₁)₂, —O—CH₂C(O)—N(R₁)₂, —O—CH₂—CH₂—CH₂—NH₂, —O—(CH₂)₂—O—(CH₂)₂—N(R₁)₂ or —O—CH₂—N(H)—C(═NR₁)(N(R₁)₂);
wherein each R₁ is, independently, H, C₁-C₁₂ alkyl, a protecting group, or substituted or unsubstituted C₁-C₁₂ alkyl, C₂-C₁₂ alkenyl, or C₂-C₁₂ alkynyl, wherein the substituent groups are halogen, hydroxyl, amino, azido, cyano, haloalkyl, alkenyl, alkoxy, thioalkoxy, haloalkoxy, or aryl.

53. The composition of claim 52 wherein each of the 2′-substituent groups is, independently, —F, —O—CH₂CH₂—O—CH₃, —O—CH₃, —O—CH₂—CH═CH₂, —O—CF₃ or —O—CH₂—CH—CH₂—NH(Rⱼ) where Rⱼ is H or C₁-C₁₀ alkyl.

54. The composition of claim 53 wherein each of the 2′-substituent groups is, independently, F, —O—CH₃, —O—CF₃, or —O—CH₂CH₂—O—CH₃.

55. The composition of claim 23 wherein at least one modified nucleotide base is a locked nucleic acid (LNA).

56. The composition of claim 23 wherein the nucleotide sugar modification is, independently, C₁-C₂₀ alkyl, C₂-C₂₀ alkenyl, C₂-C₂₀ alkynyl, C₅-C₂₀ aryl, —O-alkyl, —O-alkenyl, —O-alkynyl, —O-alkylamino, —O-alkylalkoxy, —O-alkylaminoalkyl, —O-alkyl imidazole, —OH, —SH, —S-alkyl, —S-alkenyl, —S-alkynyl, —N(H)-alkyl, —N(H)-alkenyl, —N(H)-alkynyl, —N(alkyl)₂, —O-aryl, —S-aryl, —NH-aryl, —O-aralkyl, —S-aralkyl, —N(H)-aralkyl, phthalimido (attached at N), halogen, amino, keto (—C(═O)—Ra), carboxyl (—C(═O)OH), nitro (—NO₂), nitroso (—N═O), cyano (—CN), trifluoromethyl (—CF₃), trifluoromethoxy (—O—CF₃), imidazole, azido (—N₃), hydrazino (—N(H)—NH₂), aminooxy (—O—NH₂), isocyanato (—N═C═O), sulfoxide (—S(═O)—Rₐ), sulfone (—S(═O)₂—Rₐ), disulfide (—S—S—Rₐ), silyl, heterocyclyl, carbocyclyl, an intercalator, a reporter group, a conjugate group, polyamine, polyamide, polyalkylene glycol, or a polyether of the formula (—O-alkyl)ₘₐ;
wherein each Rₐ is, independently, hydrogen, a protecting group, or substituted or unsubstituted alkyl, alkenyl, or alkynyl, wherein the substituent groups are haloalkyl, alkenyl, alkoxy, thioalkoxy, haloalkoxy, aryl, halogen, hydroxyl, amino, azido, carboxy, cyano, nitro, mercapto, a sulfide group, a sulfonyl group, or a sulfoxide group;
or each sugar substituent group has one of formula Ia or IIa: 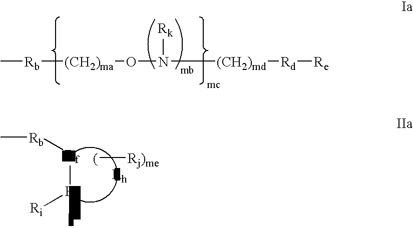
wherein:
R_{b} is O, S or NH;
R_{d} is a single bond, O, S or C(═O);
Rₑ is C₁-C₁₀ alkyl, N(Rₖ)(Rₘ), N(Rₖ)(Rₙ), N═C(Rₚ)(R_{q}), N═C(Rₚ)(Rᵣ) or has formula IIIa; 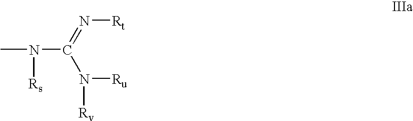
Rₚ and R_{q} are each, independently, hydrogen or C₁-C₁₀ alkyl;
Rᵣ is —Rₓ-R_{y};
each Rₛ, Rₜ, Rᵤ and Rᵥ is, independently, hydrogen, C(O)R_{w}, substituted or unsubstituted C₁-C₁₀ alkyl, substituted or unsubstituted C₂-C₁₀ alkenyl, substituted or unsubstituted C₂-C₁₀ alkynyl, alkylsulfonyl, arylsulfonyl, a chemical functional group, or a conjugate group, wherein the substituent groups are hydroxyl, amino, alkoxy, carboxy, benzyl, phenyl, nitro, thiol, thioalkoxy, halogen, alkyl, aryl, alkenyl, or alkynyl;
or, optionally, Rᵤ and Rᵥ, together form a phthalimido moiety with the nitrogen atom to which they are attached;
each R_{w} is, independently, substituted or unsubstituted C₁-C₁₀ alkyl, trifluoromethyl, cyanoethyloxy, methoxy, ethoxy, t-butoxy, allyloxy, 9-fluorenylmethoxy, 2-(trimethylsilyl)-ethoxy, 2,2,2-trichloroethoxy, benzyloxy, butyryl, iso-butyryl, phenyl, or aryl;
Rₓ is a bond or a linking moiety;
R_{y} is a chemical functional group, a conjugate group or a solid support medium;
Rₖ is hydrogen, a nitrogen protecting group or —Rₓ-R_{y};
each Rₘ and Rₙ, is, independently, H, a nitrogen protecting group, substituted or unsubstituted C₁-C₁₀ alkyl, substituted or unsubstituted C₂-C₁₀ alkenyl, substituted or unsubstituted C₂-C₁₀ alkynyl, wherein the substituent groups are hydroxyl, amino, alkoxy, carboxy, benzyl, phenyl, nitro, thiol, thioalkoxy, halogen, alkyl, aryl, alkenyl, alkynyl, NH3+, N(Rᵤ)(Rᵥ) guanidine, or acyl where the acyl is an acid amide or an ester;
or Rₘ and Rₙ, together, are a nitrogen protecting group, are joined in a ring structure that optionally includes an additional heteroatom selected from N and O, or are a chemical functional group;
Rᵢ is OR_{z}, SR_{z}, or N(R_{z})₂;
each R_{z} is, independently, H, C₁-C₈ alkyl, C₁-C₈ haloalkyl, C(═NH)N(H)Rᵤ, C(═O)N(H)Rᵤ or OC(═O)N(H)Rᵤ;
R_{f}, R_{g} and Rₕ comprise a ring system having from about 4 to about 7 carbon atoms or having from about 3 to about 6 carbon atoms and 1 or 2 heteroatoms wherein the heteroatoms are oxygen, nitrogen, or sulfur and wherein the ring system is aliphatic, unsaturated aliphatic, aromatic, or saturated or unsaturated heterocyclic;
Rⱼ is alkyl or haloalkyl having 1 to about 10 carbon atoms, alkenyl having 2 to about 10 carbon atoms, alkynyl having 2 to about 10 carbon atoms, aryl having 6 to about 14 carbon atoms, N(Rₖ)(Rₘ) ORₖ, halo, SRₖ or CN;
ma is 1 to about 10;
each mb is, independently, 0 or 1;
mc is 0 or an integer from 1 to 10;
md is an integer from 1 to 10;
me is from 0, 1 or 2; and
provided that when mc is 0, md is greater than 1.
